(19) United States
(12) Patent Application Publication
(10) Pub. No.: US 2025/0163024 A1
(43) Pub. Date: May 22, 2025

(54) CHEMICAL TARGETING SWI/SNF RELATED, MATRIX ASSOCIATED, ACTIN DEPENDENT REGULATOR OF CHROMATIN, SUBFAMILY A, MEMBER 4 (SMARCA4) AND USE IN DIFFUSE INTRINSIC PONTINE GLIOMA (DIPG)

(71) Applicant: DANA-FARBER CANCER INSTITUTE, INC., Boston, MA (US)

(72) Inventors: Jun QI, Sharon, MA (US); Mariella FILBIN, Boston, MA (US); Yu LIANG, Boston, MA (US); Eshini A. PANDITHARATNA, Boston, MA (US)

(73) Assignee: DANA-FARBER CANCER INSTITUTE, INC., Boston, MA (US)

(21) Appl. No.: 18/725,441

(22) PCT Filed: Jan. 6, 2023
(86) PCT No.: PCT/US2023/010301

§ 371 Date: Jun. 28, 2024

Related U.S. Application Data
(60) Provisional application No. 63/297,505, filed on Jan. 7, 2022.

(51) Int. Cl. C07D 401/14; A61K 45/06; A61K 47/55

(52) U.S. Cl. CPC C07D 401/14; A61K 45/06; A61K 47/55

(57) ABSTRACT

The present disclosure relates to bifunctional compounds comprising targeting moiety that binds SWI/SNF related, matrix associated, actin dependent regulator of chromatin, subfamily A, member 4 (SMARCA4) or SMARCA2 and SMARCA4, and a degron that binds an E3 ubiquitin ligase. The disclosure further provides compositions and methods for treating diseases or conditions mediated by aberrant activity of SMARCA4 or SMARCA2 and SMARCA4, including diffuse midline glioma harboring a H3K27M mutation.

### RELATED APPLICATIONS

This application claims the benefit of priority under 35 U.S.C. § 119(e) to U.S. Provisional Application No. 63/297,505, filed on Jan. 7, 2022, which is incorporated herein by reference in its entirety.

### BACKGROUND

Diffuse intrinsic pontine glioma (DIPG) and diffuse midline glioma are uniformly fatal pediatric central nervous system cancers, refractory to standard of care therapeutic modalities (Hoffman et al., J Clin Oncol 36:1963-1972 (2018)). The primary genetic drivers are a set of recurrent amino acid substitutions in genes encoding histone H3 (H3.3 and H3.1) wherein lysine 27 is mutated to methionine (H3K27M) (Schwartzentruber et al., Nature 482:226-231 (2012); Wu et al., Nat Genet 44:251-253 (2012)). The H3K27M oncohistones do not contain direct enzymatic function, and are therefore undruggable.

### SUMMARY

A first aspect of the present disclosure is directed to a bifunctional compound having a structure represented by formula (I):

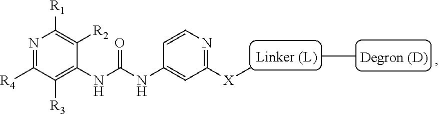

or a pharmaceutically acceptable salt or stereoisomer thereof, wherein

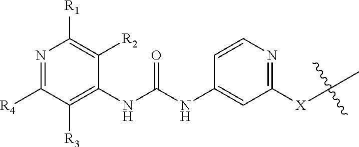

represents a targeting moiety that binds SWI/SNF related, matrix associated, actin dependent regulator of chromatin, subfamily a, member 4 (SMARCA4) or SMARCA2 and SMARCA4, the degron represents a moiety that binds an E3 ubiquitin ligase and L represents a linker that covalently attaches the targeting moiety to the degron, wherein:
R₁ and R₄ are each independently H or halogen;
R₂ is H or NH₂;
R₃ is H, NH₂, NO₂, OH, CN, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
X is C(O), C(O)NH,

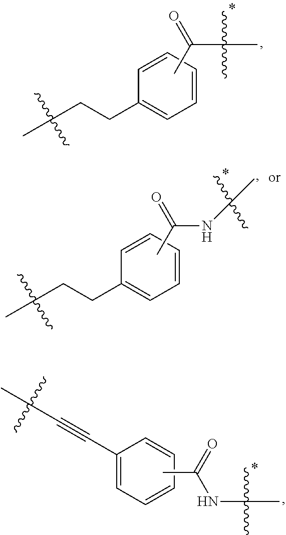

wherein the squiggle ( [symbol] ) is the point of attachment to the pyridine ring and the squiggle-asterisk ( [symbol] ) is the point of attachment to the linker.

Another aspect of the present disclosure is directed to a pharmaceutical composition containing a therapeutically effective amount of a bifunctional compound of formula (I) or a pharmaceutically acceptable salt or stereoisomer thereof, and a pharmaceutically acceptable carrier.

In another aspect of the present disclosure, methods of making the bifunctional compounds are provided.

A further aspect of the present disclosure is directed to a method of treating a disease or disorder characterized or mediated by aberrant activity of SMARCA4 or SMARCA2 and SMARCA4 (SMARCA2/4), that includes administering to a subject in need thereof a therapeutically effective amount of the bifunctional compound of formula (I) or a pharmaceutically acceptable salt or stereoisomer thereof.

In some embodiments, the disease or disorder is cancer. In some embodiments, the cancer is selected from synovial sarcoma, lung cancer, ovarian cancer, brain cancer, kidney cancer, leukemia, non-small cell lung cancer, Burkitt's Lymphoma, childhood medulloblastoma, pancreatic adenocarcinoma, ovarian clear cell carcinoma, renal cell carcinoma, endometrial carcinoma, and melanoma.

Another aspect of the present disclosure is directed to a method of treating a diffuse midline glioma harboring a H3K27M mutation, comprising administering to a subject in need thereof a therapeutically effective amount of the bifunctional compound of formula (I) or pharmaceutically acceptable salt or stereoisomer thereof, or a bifunctional compound of formula I′, I″, or II″, or a pharmaceutically acceptable salt or stereoisomer thereof.

In some embodiments, the diffuse midline glioma harboring the H3K27M mutation is diffuse intrinsic pontine glioma (DIPG).

Without intending to be bound by any particular theory of operation, the bifunctional compounds of the present disclosure may serve as a set of new chemical tools for SMARCA4 or SMARCA2 and SMARCA4 knockdown and may provide effective treatments for SMARCA4 or SMARCA2/4-mediated diseases and disorders such as cancer. Applicant has surprisingly and unexpectedly discovered that DIPG cells are sensitive to SMARCA4 depletion. The degradation SMARCA4, a catalytic ATPase subunit of the SWItch/Sucrose Non-fermentable (SWI/SNF) complex, also known the Brg/Brm-associated (BAF) complex, effectively disrupts the function of the SWI/SNF or BAF complex. Therefore, disclosed bifunctional compounds may provide effective treatments for DIPG and other diffuse midline gliomas harboring a H3K27M mutation.

### BRIEF DESCRIPTION OF THE DRAWINGS

FIG. 1A is a western blot showing SWI/SNF related, matrix associated, actin dependent regulator of chromatin, subfamily a, member 4 (SMARCA4), also known as Brahma-related gene 1 (BRG1) or BRG herein), degradation in NOMO-1 cells with bifunctional compounds 1-3 and DMSO (negative control).

FIG. 1B is a western blot showing SMARCA4 (BRG) degradation in NOMO-1 cells with bifunctional compounds 4-6 and DMSO (negative control).

### DETAILED DESCRIPTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present disclosure, and to the extent the following terms are used herein to further describe them, the following definitions apply.

As used herein, the term “alkyl” refers to a saturated linear or branched-chain monovalent hydrocarbon radical. To the extent not described otherwise for any one or more groups, in one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. To the extent not described otherwise for any one or more groups, in some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. To the extent not described otherwise for any one or more groups, in one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. To the extent not described otherwise for any one or more groups, in some embodiments, the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, to the extent not described otherwise for any one or more groups, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. To the extent not described otherwise for any one or more groups, in one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. To the extent not described otherwise for any one or more groups, in one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring. The structure of any aryl group that is capable of having double bonds positioned differently is considered so as to embrace any and all such resonance structures.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}—aryl where R^{c} is an alkylene chain such as methylene or ethylene.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. To the extent not described otherwise for any one or more groups, in some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

To the extent not described otherwise for any one or more groups, in some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur and oxygen. In some embodiments, heterocyclyl includes 4- to 6-membered monocycles having one or more heteroatoms selected from nitrogen, sulfur and oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. To the extent not described otherwise for any one or more groups, in one embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring. The structure of any heteroaryl group that is capable of having double bonds positioned differently is considered to embrace any and all such resonance structures.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

To the extent not disclosed otherwise for any particular group(s), representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁—C), substituted alkylthio (e.g., C₁—C), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

The term “binding” as it relates to interaction between the targeting ligand and the targeted proteins, which in this disclosure are SWI/SNF related, matrix associated, actin dependent regulator of chromatin, subfamily a, member 4 (SMARCA4) or SMARCA2 and SMARCA4, typically refers to an inter-molecular interaction that is preferential (also referred to herein as “selective”) in that binding of the targeting ligand with other proteins present in the cell is substantially less and may be functionally insignificant. The terms “selective” and “selectivity” refer to the ability of the bifunctional compound to discriminate between and among molecular targets.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but is sufficient nonetheless to achieve recruitment of the ligase to the targeted proteins, which in this disclosure are SMARCA4 or SMARCA2 and SMARCA4, for selective degradation.

Broadly, the bifunctional compounds comprise a structure represented by formula (I):

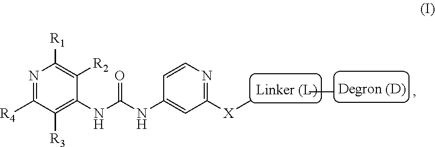

or a pharmaceutically acceptable salt or stereoisomer thereof, wherein

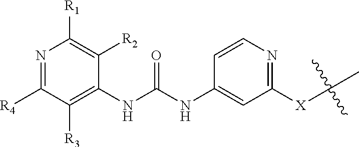

represents a targeting moiety that binds SWI/SNF related, matrix associated, actin dependent regulator of chromatin, subfamily a, member 4 (SMARCA4) or both SMARCA2 and SMARCA4, the degron represents a moiety that binds an E3 ubiquitin ligase and L represents a linker that covalently attaches the targeting moiety to the degron, wherein:
R₁ and R₄ are each independently H or halogen;
R₂ is H or NH₂;
R₃ is H, NH₂, NO₂, OH, CN, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
X is C(O), C(O)NH,

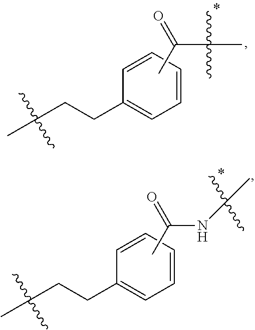

or, wherein the squiggle ( [symbol] ) is the point of attachment to the pyridine ring and the squiggle-asterisk ( [symbol] ) is the point of attachment to the linker.

In some embodiments, R₃ is H, NH₂, or CH₂OH.

In some embodiments, R₁ is halogen, and R₂, R₃, R₄ are H.

In some embodiments, R₁ is halogen, R₂ and R₄ are H, and R₃ is CH₂OH.

In some embodiments, R₁ is Cl or F.

In some embodiments, R₁ and R₃ are H, R₂ is NH₂, and R₄ is halogen.

In some embodiments, R₄ is C₁.

In some embodiments, wherein X is C(O), C(O)NH,

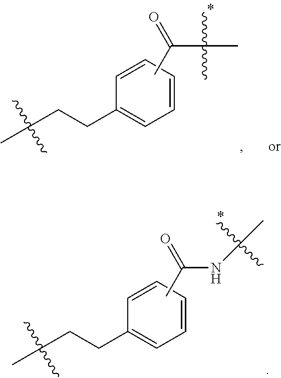

In some embodiments, X is

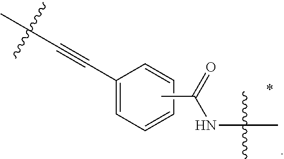

In some embodiments, wherein R₁ is halogen and R₃ is H, NH₂, or methoxy, and X is not

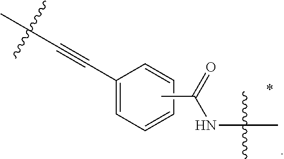

In some embodiments, bifunctional compounds of the disclosure are represented any one of structures I-1 to I-17:

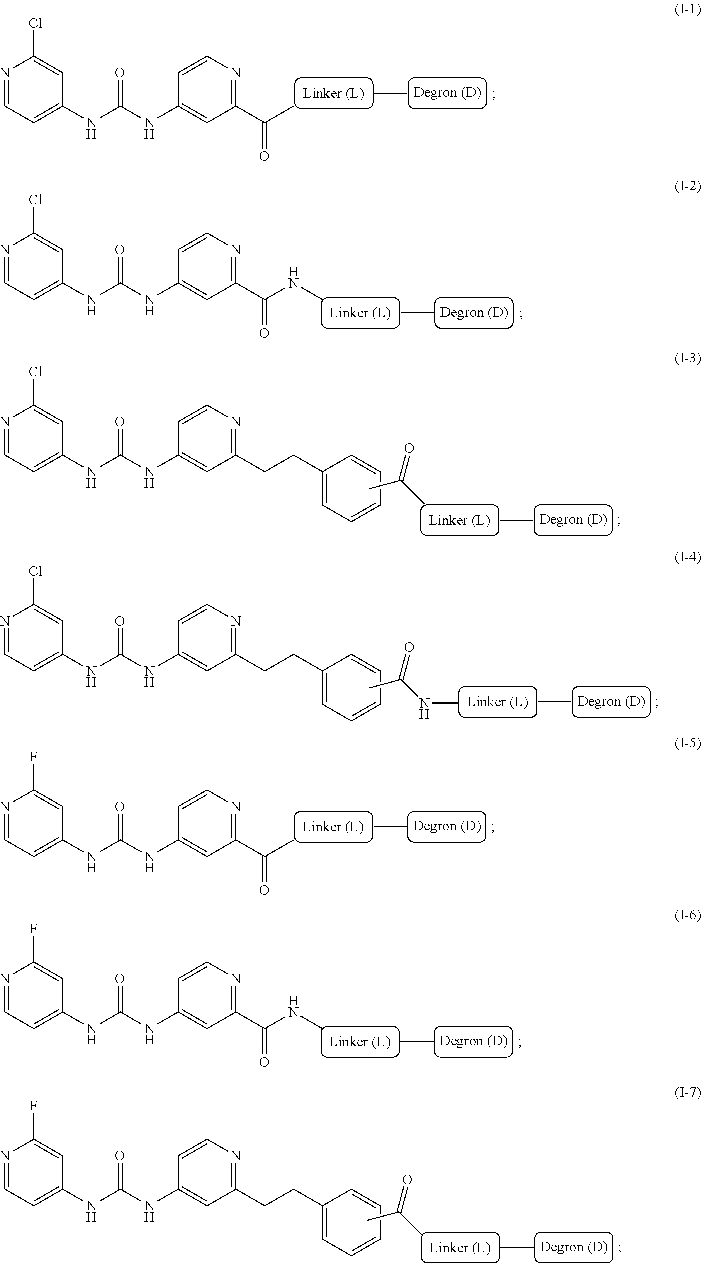

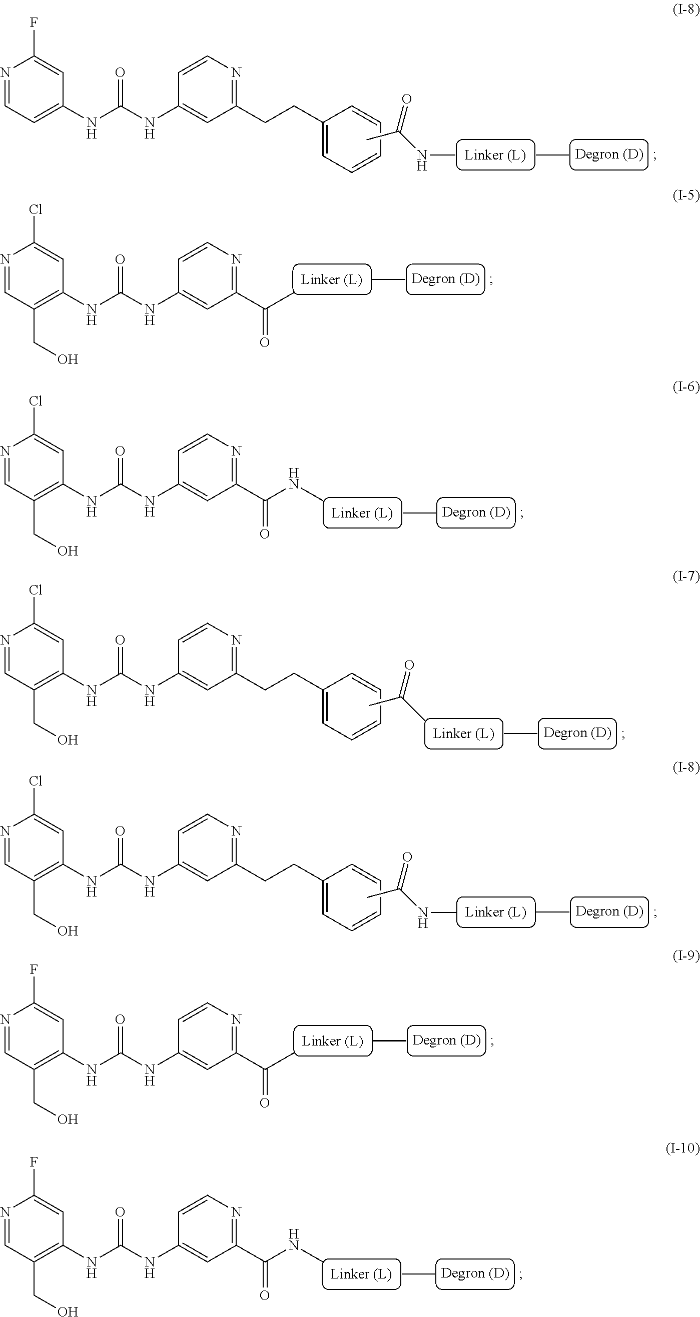

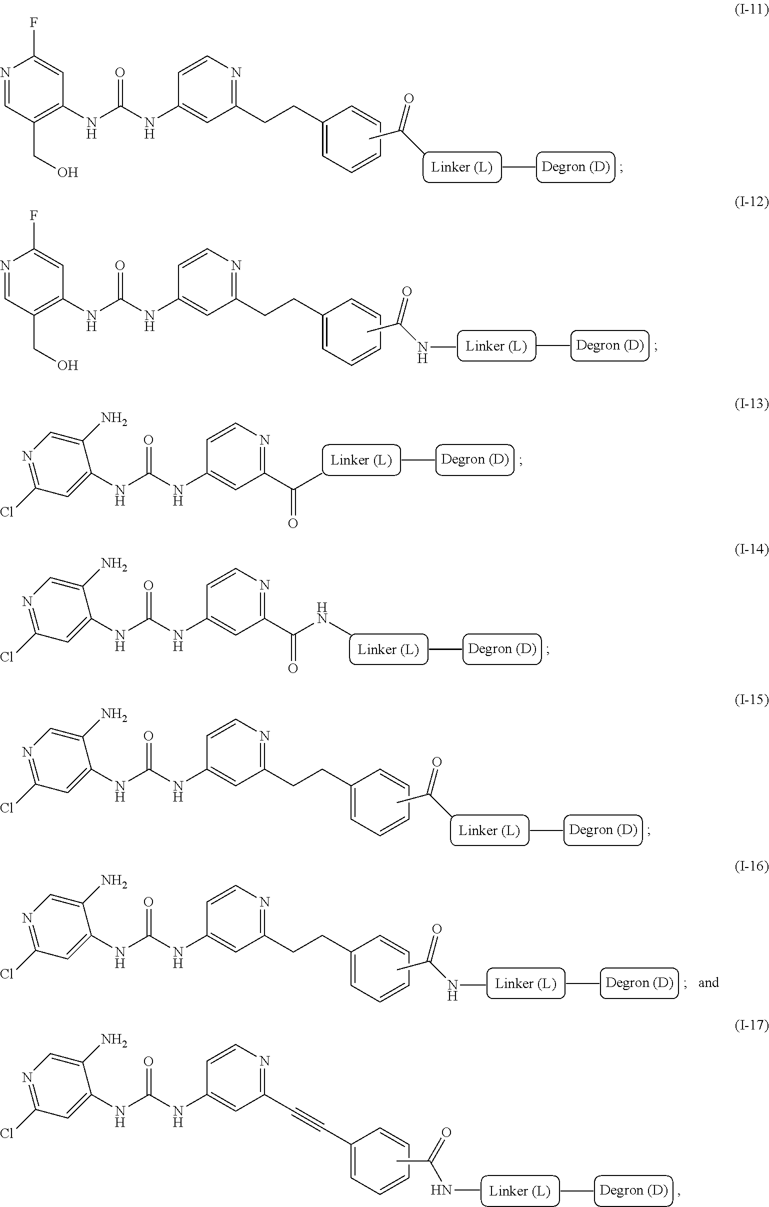

or a pharmaceutically acceptable salt or stereoisomer thereof.

### Linkers

The linker (“L”) provides a covalent attachment between the targeting ligand and the degron. The structure of linker may not be critical, provided it is substantially non-interfering with the activity of

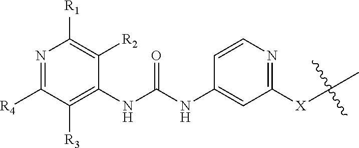

or the degron. In some embodiments, the linker includes an alkylene chain (e.g., having 1-20 alkylene units). In some embodiments, the linker comprises an uninterrupted C₂ to C₁₅ alkylene chain. In other embodiments, the linker may include an alkylene chain, which may be interrupted by, and/or terminate (at either or both termini) at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may include a C₂-C₁₅ alkylene chain terminating in NH-group wherein the nitrogen is also bound to the degron.

In some embodiments, the linker includes an alkylene chain having 2-10 alkylene units that is interrupted by and/or terminating in

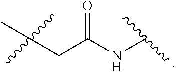 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of alkylene linkers that may be suitable for use in the present disclosure include the following:

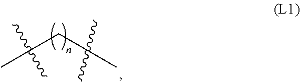

wherein n is an integer of 1-12 (“of” meaning inclusive), e.g., 1-12, 1-11, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10, examples of which include:

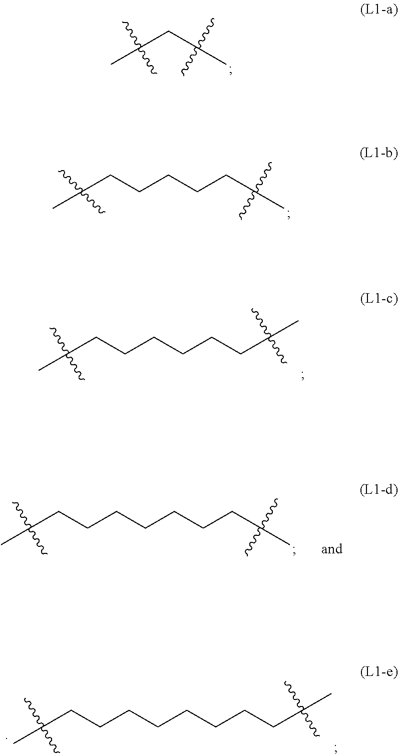

alkylene chains terminating in various functional groups (as described above), examples of which are as follows:

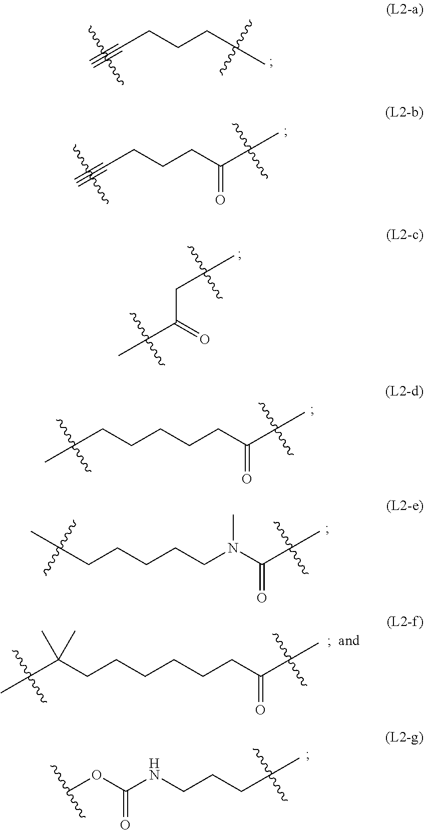

alkylene chains interrupted with various functional groups (as described above), examples of which are as follows:

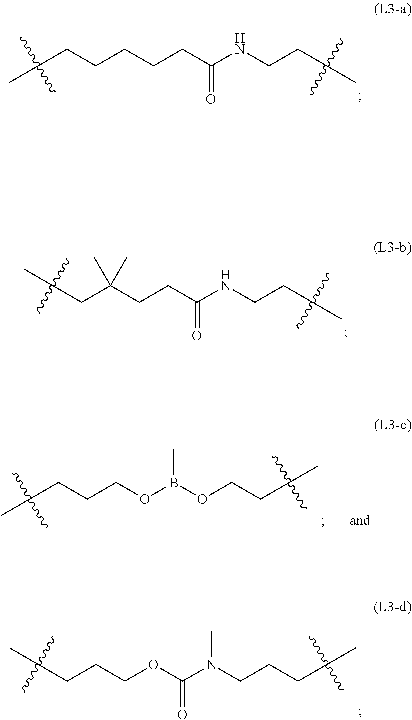 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with a heterocyclene group, e.g.,

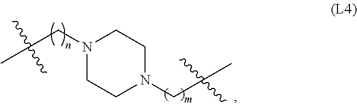

wherein m and n are independently integers of 0-10, examples of which include:

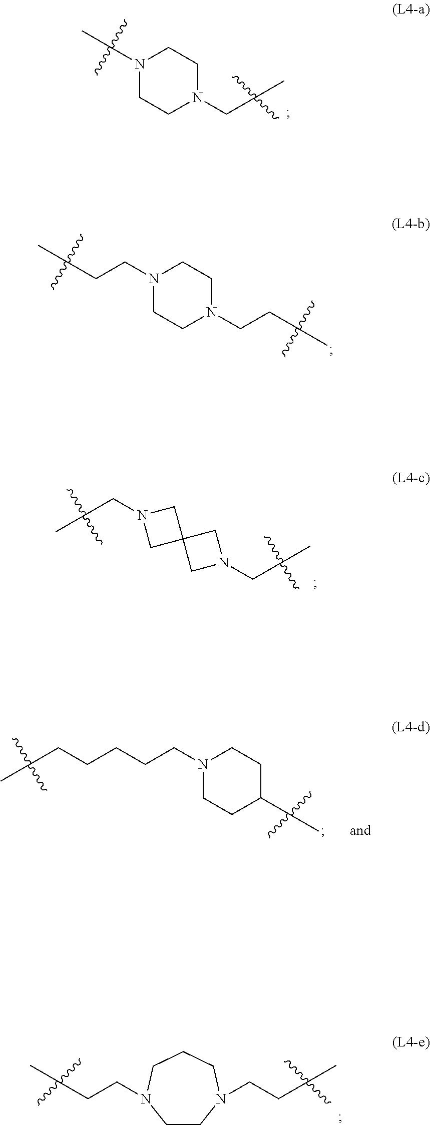 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by an amide, a heterocyclene and/or an aryl group, examples of which include:

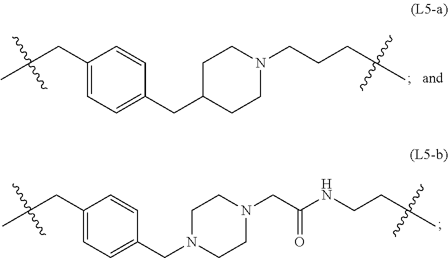 [SMILES: *CCCN1CCC(Cc2ccc(C*)cc2)CC1 ; *CCNC(=O)CN1CCN(Cc2ccc(C*)cc2)CC1]

alkylene chains interrupted by a heterocyclene, an aryl group, and a heteroatom, examples of which include:

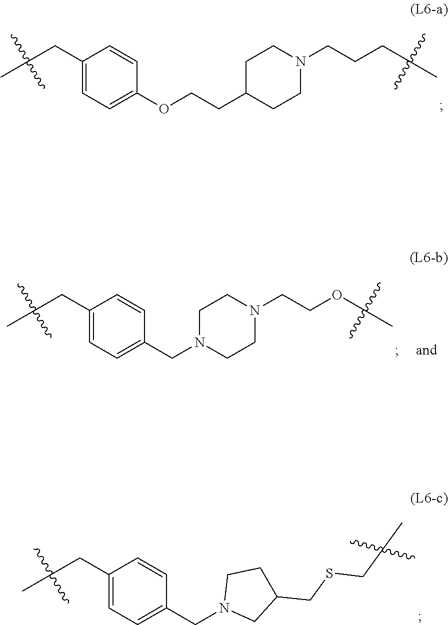 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

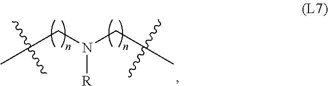

wherein each n is independently an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H or C₁ to C₄ alkyl, an example of which is

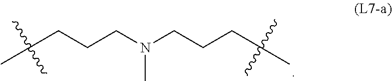 [SMILES: *CCCN(C)CCC*]

In some embodiments, the linker comprises a C₆ carbocyclene, 6-membered heterocyclene, or phenyl group. In some embodiments, the 6-membered heterocyclene is

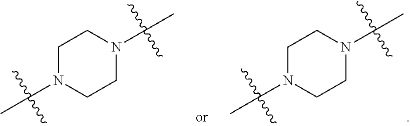 [SMILES: *N1CCN(*)CC1 ; *N1CCN(*)CC1]

In some embodiments, the linker may include a polyethylene glycol chain that may terminate (at either or both termini) in at least one of —S—, —N(R′)—, —C≡C—, —C(O)—, —C(O)O——OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In some embodiments, the linker includes a polyethylene glycol chain having 2-8 PEG units and terminates at one or both termini in

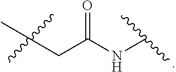 [SMILES: *CC(=O)N*]

In some embodiments, the linker includes 1-6 uninterrupted PEG units.

Representative examples of linkers that include a polyethylene glycol chain include:

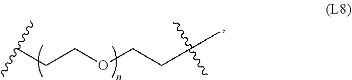

wherein n is an integer of 1-10, examples of which include:

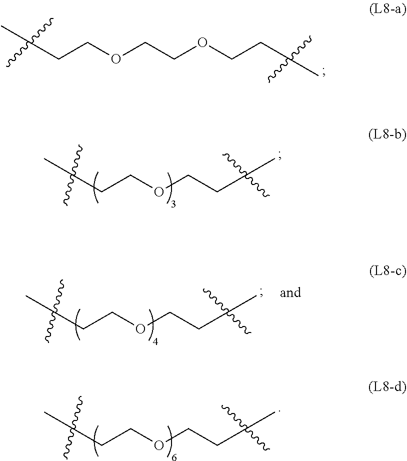

In some embodiments, the polyethylene glycol linker may terminate in a functional group, examples of which are as follows:

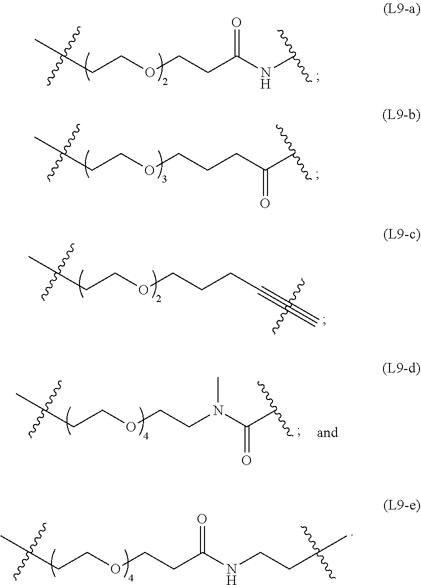

In some embodiments, the linker is represented by any one of structures:

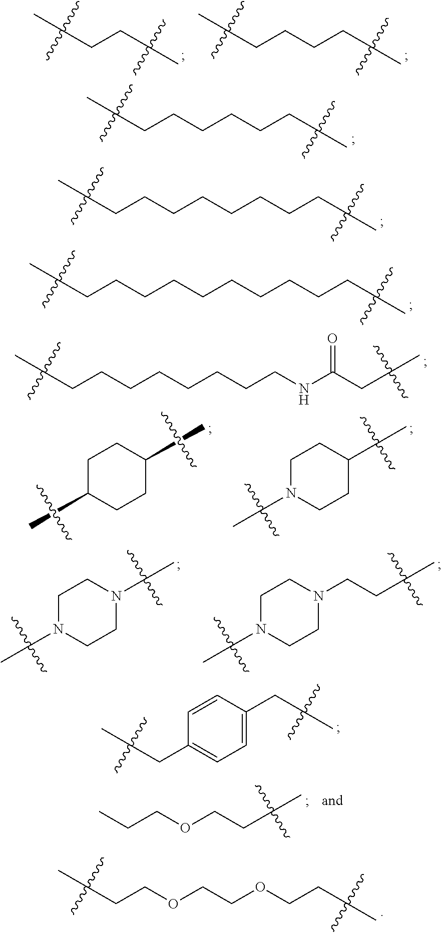

### Degrons

The Ubiquitin-Proteasome Pathway (UPP) is a critical cellular pathway that regulates key regulator proteins and degrades misfolded or abnormal proteins. UPP is central to multiple cellular processes. The covalent attachment of ubiquitin to specific protein substrates is achieved through the action of E3 ubiquitin ligases. These ligases include over 500 different proteins and are categorized into multiple classes defined by the structural element of their E3 functional activity.

In some embodiments, the degron binds the E3 ligase which is cereblon. (CRBN).

Representative examples of such degrons are represented by any one of structures (D1a) to (D1d):

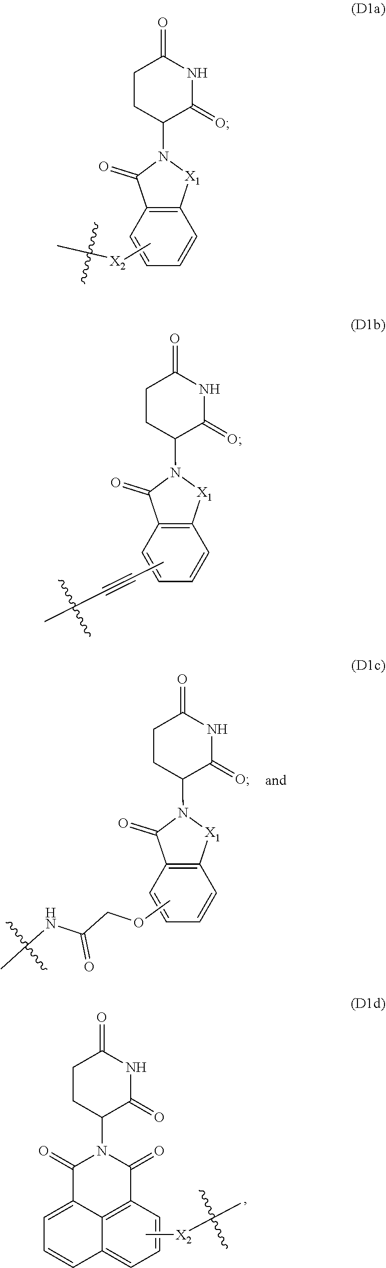

wherein X₁ is CH₂ or C(O) and X₂ is a bond, CH₂, NH, or O.

Yet other degrons that bind cereblon and which may be suitable for use in the present disclosure are disclosed in U.S. Pat. No. 9,770,512, and U.S. Patent Application Publication Nos. 2018/0015087, 2018/0009779, 2016/0243247, 2016/0235731, 2016/0235730, and 2016/0176916, and International Patent Publications WO 2017/197055, WO 2017/197051, WO 2017/197036, WO 2017/197056 and WO 2017/197046.

In some embodiments, the degron binds the von Hippel-Lindau (VHL) E3 ubiquitin ligase.

Representative examples of such degrons are represented by any one of structures (D1-a) to (D1-f):

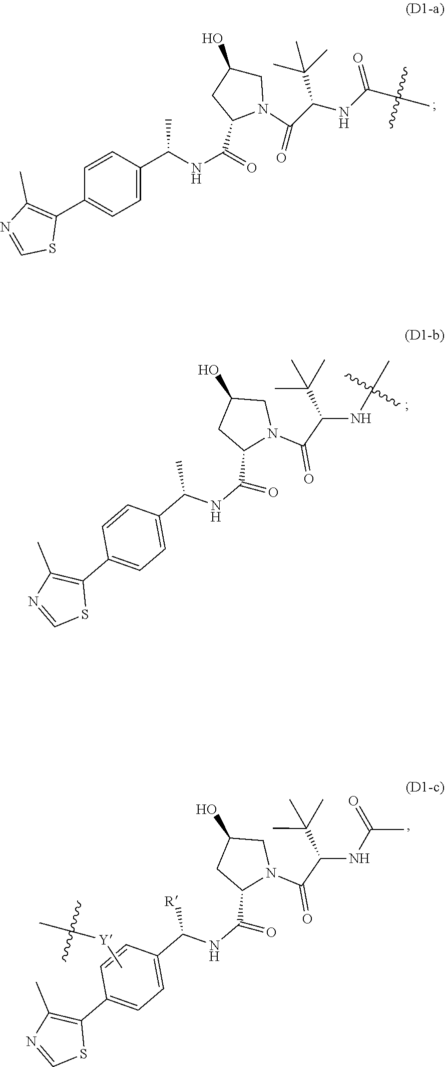

wherein Y′ is a bond, N, O or C and R′ is H or methyl;

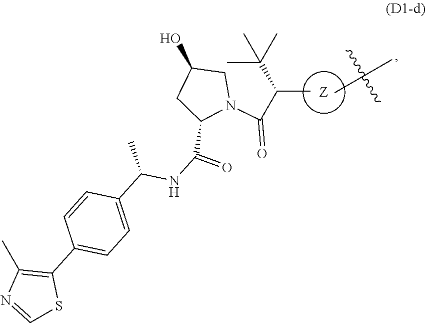

wherein Z is a C₅-C₆ carbocyclic or a C₅-C₆ heterocyclic group;

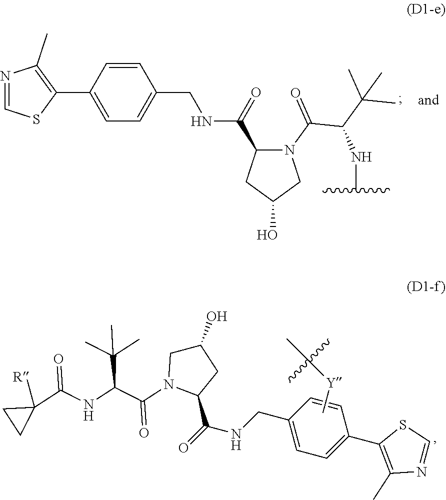

wherein Y″ is a bond, N, O or C and R″ is F or CN,
or a stereoisomer thereof

In some embodiments, Z is

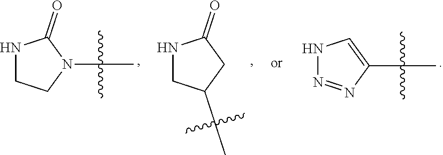 [SMILES: *N1CCNC1=O ; *C1CNC(=O)C1 ; *c1c[nH]nn1]

Yet other degrons that bind VHL and which may be suitable for use in the present disclosure are disclosed in U.S. Patent Application Publication 2017/0121321 A1.

In some embodiments, bifunctional compounds of the present disclosure may be represented by any one of structures (1) to (27):

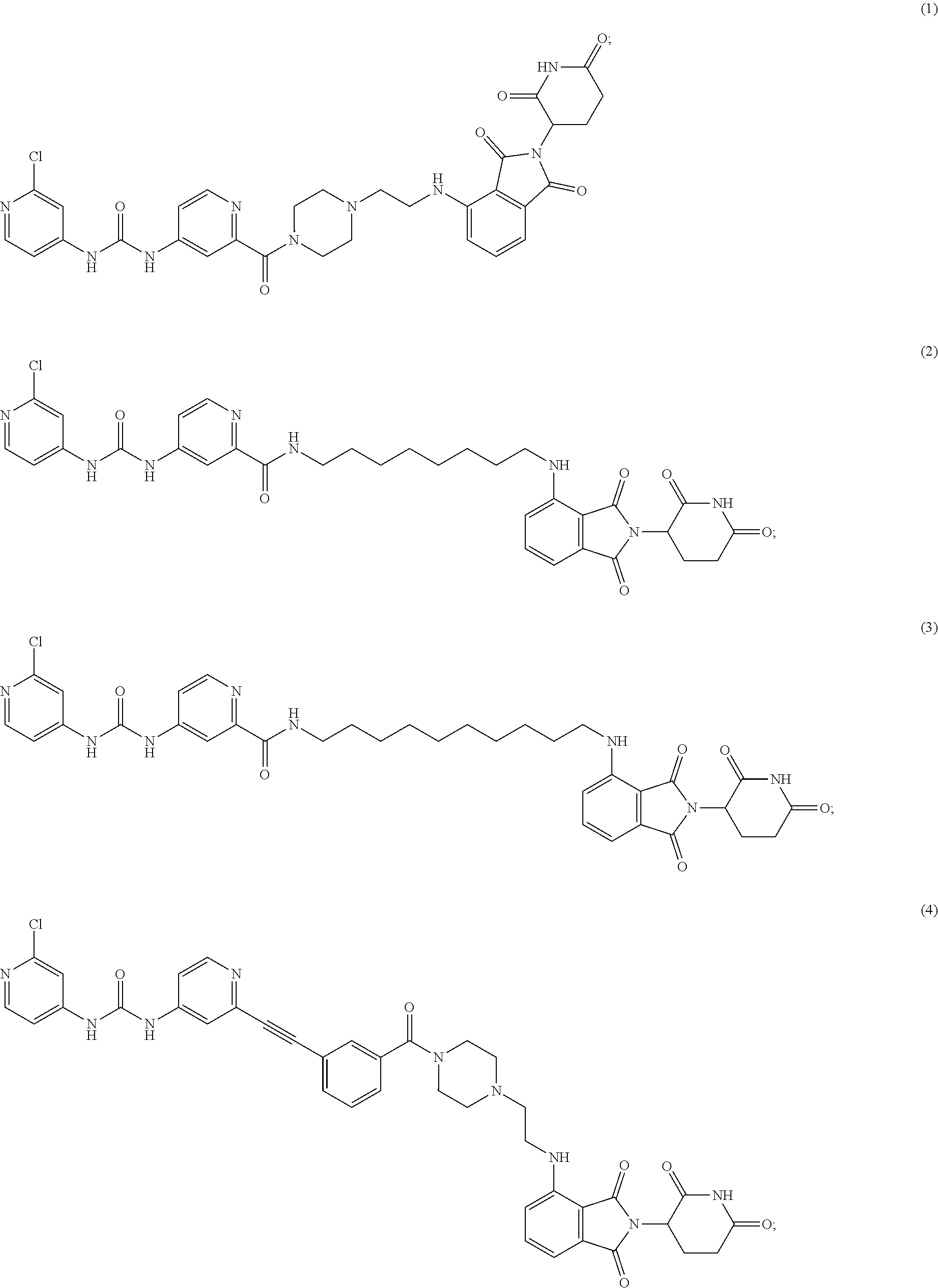 [SMILES: O=C1CCC(N2C(=O)c3cccc(NCCN4CCN(C(=O)c5cc(NC(=O)Nc6ccnc(Cl)c6)ccn5)CC4)c3C2=O)C(=O)N1 ; O=C1CCC(N2C(=O)c3cccc(NCCCCCCCCNC(=O)c4cc(NC(=O)Nc5ccnc(Cl)c5)ccn4)c3C2=O)C(=O)N1 ; O=C1CCC(N2C(=O)c3cccc(NCCCCCCCCCCNC(=O)c4cc(NC(=O)Nc5ccnc(Cl)c5)ccn4)c3C2=O)C(=O)N1 ; O=C1CCC(N2C(=O)c3cccc(NCCN4CCN(C(=O)c5cccc(C#Cc6cc(NC(=O)Nc7ccnc(Cl)c7)ccn6)c5)CC4)c3C2=O)C(=O)N1]

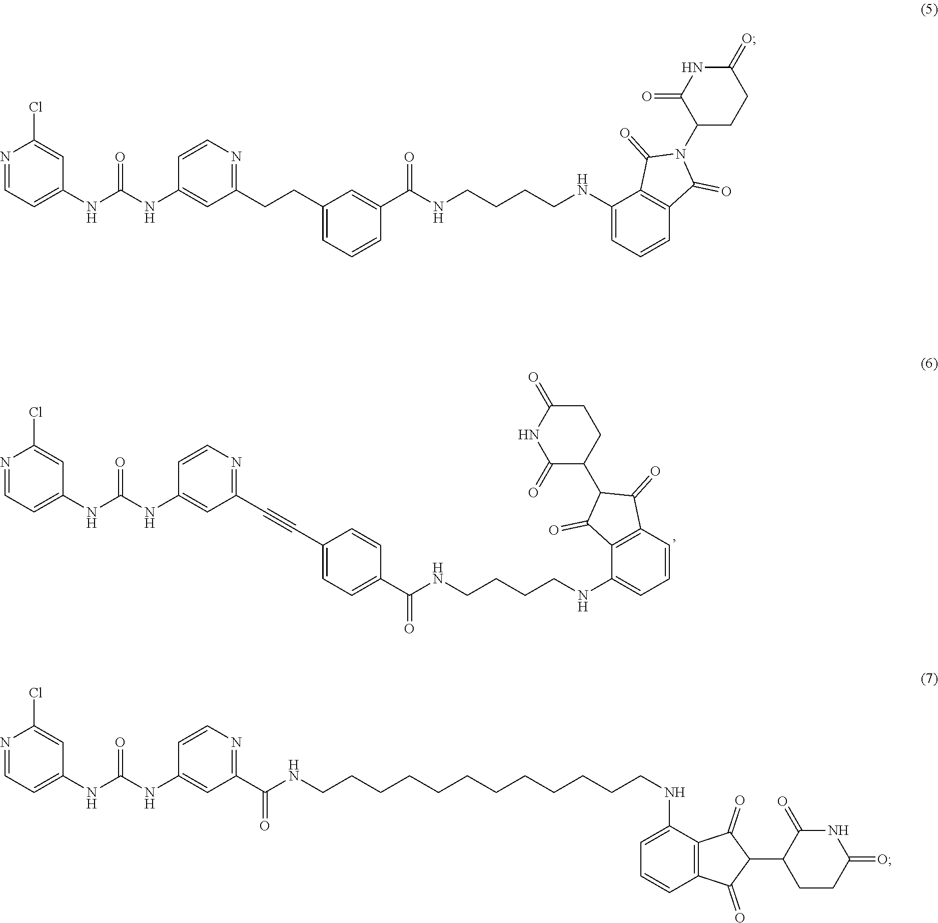 [SMILES: O=C1CCC(N2C(=O)c3cccc(NCCCCNC(=O)c4cccc(CCc5cc(NC(=O)Nc6ccnc(Cl)c6)ccn5)c4)c3C2=O)C(=O)N1 ; O=C1CCC(C2C(=O)c3cccc(NCCCCNC(=O)c4ccc(C#Cc5cc(NC(=O)Nc6ccnc(Cl)c6)ccn5)cc4)c3C2=O)C(=O)N1 ; O=C1CCC(C2C(=O)c3cccc(NCCCCCCCCCCCCNC(=O)c4cc(NC(=O)Nc5ccnc(Cl)c5)ccn4)c3C2=O)C(=O)N1]

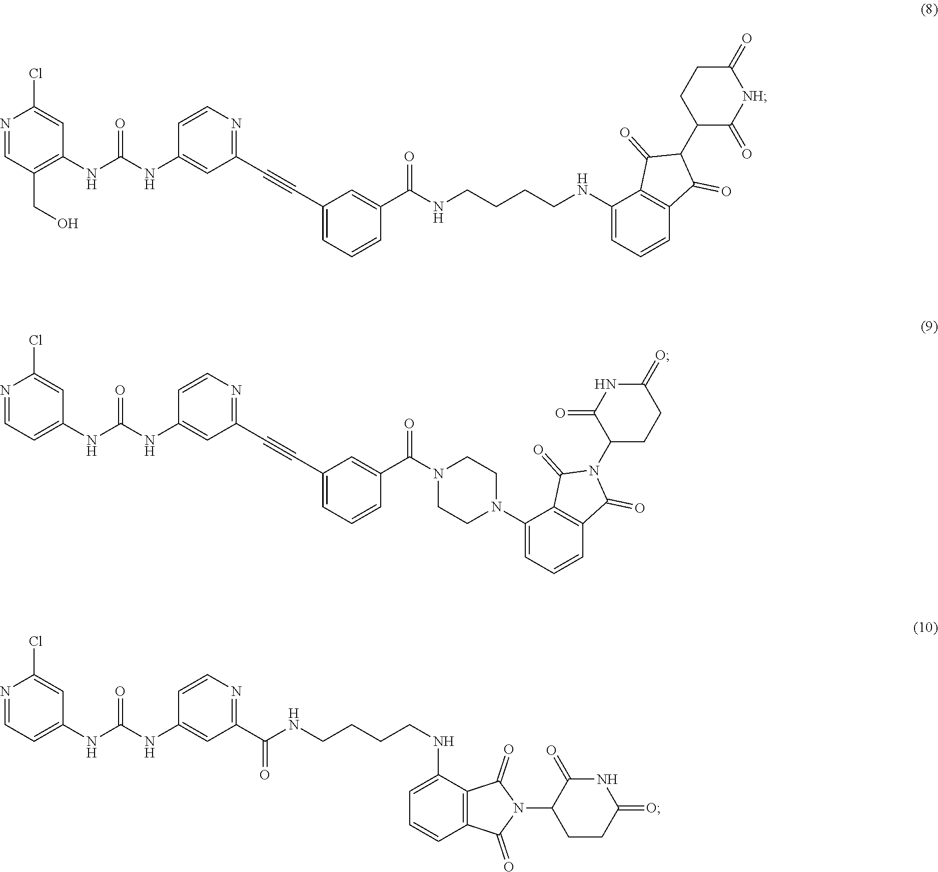 [SMILES: O=C1CCC(C2C(=O)c3cccc(NCCCCNC(=O)c4cccc(C#Cc5cc(NC(=O)Nc6cc(Cl)ncc6CO)ccn5)c4)c3C2=O)C(=O)N1 ; O=C1CCC(N2C(=O)c3cccc(N4CCN(C(=O)c5cccc(C#Cc6cc(NC(=O)Nc7ccnc(Cl)c7)ccn6)c5)CC4)c3C2=O)C(=O)N1 ; O=C1CCC(N2C(=O)c3cccc(NCCCCNC(=O)c4cc(NC(=O)Nc5ccnc(Cl)c5)ccn4)c3C2=O)C(=O)N1]

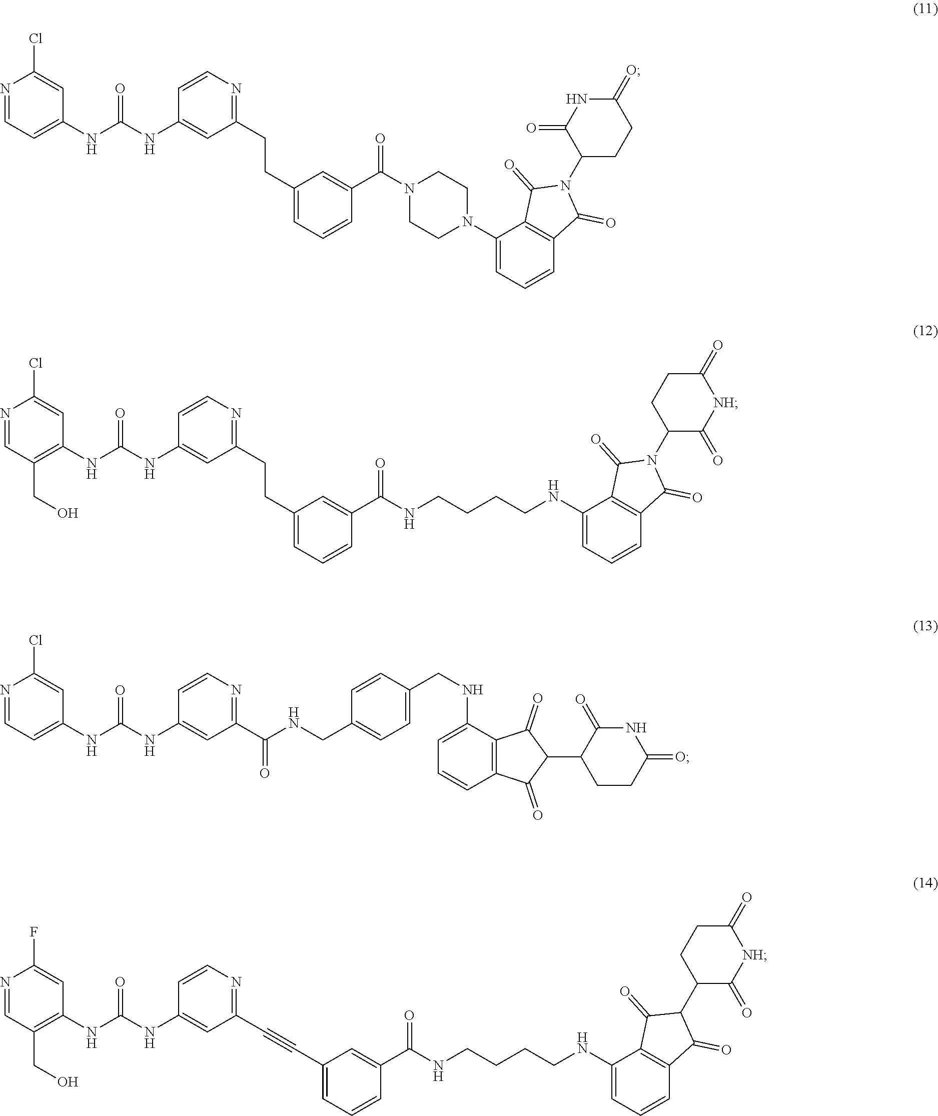 [SMILES: O=C1CCC(N2C(=O)c3cccc(N4CCN(C(=O)c5cccc(CCc6cc(NC(=O)Nc7ccnc(Cl)c7)ccn6)c5)CC4)c3C2=O)C(=O)N1 ; O=C1CCC(N2C(=O)c3cccc(NCCCCNC(=O)c4cccc(CCc5cc(NC(=O)Nc6cc(Cl)ncc6CO)ccn5)c4)c3C2=O)C(=O)N1 ; O=C1CCC(C2C(=O)c3cccc(NCc4ccc(CNC(=O)c5cc(NC(=O)Nc6ccnc(Cl)c6)ccn5)cc4)c3C2=O)C(=O)N1 ; O=C1CCC(C2C(=O)c3cccc(NCCCCNC(=O)c4cccc(C#Cc5cc(NC(=O)Nc6cc(F)ncc6CO)ccn5)c4)c3C2=O)C(=O)N1]

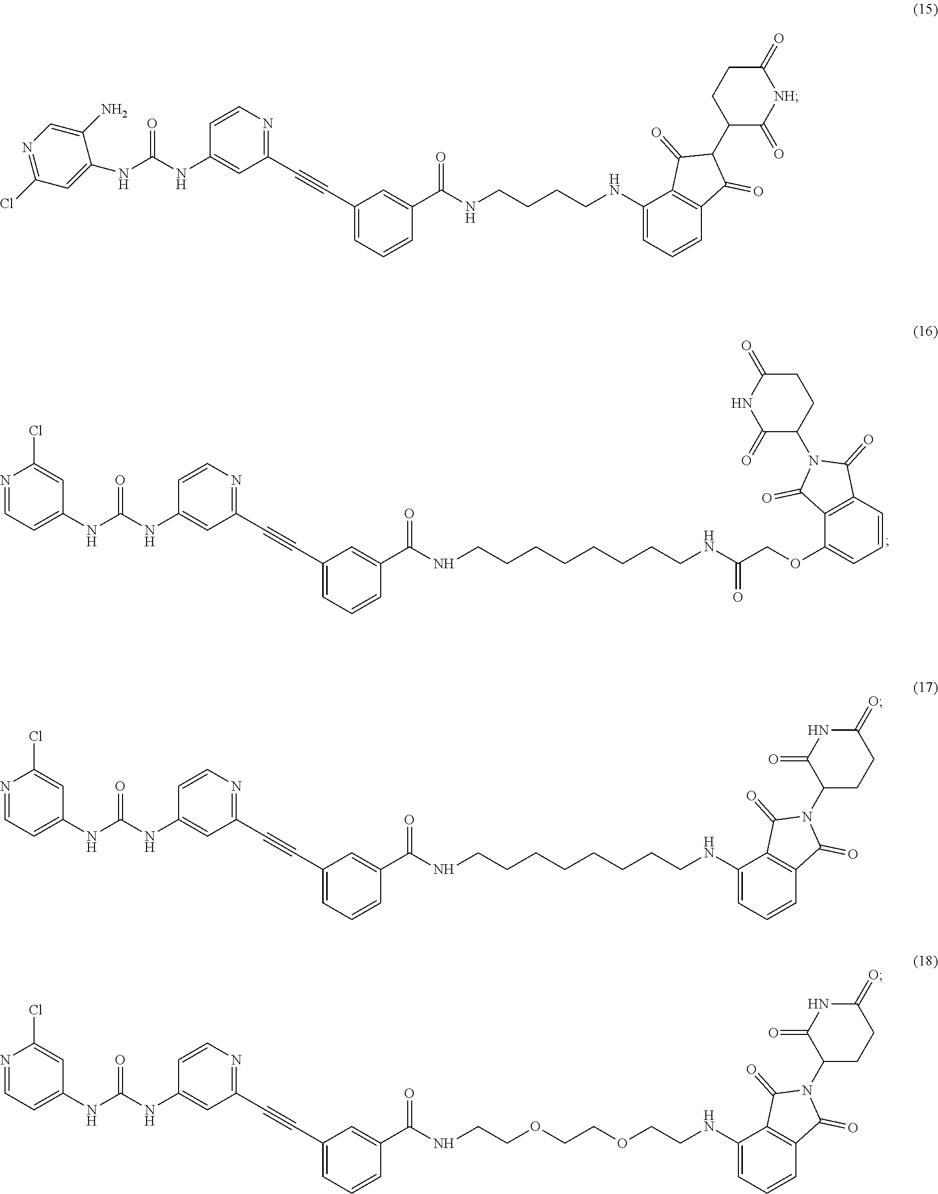 [SMILES: Nc1cnc(Cl)cc1NC(=O)Nc1ccnc(C#Cc2cccc(C(=O)NCCCCNc3cccc4c3C(=O)C(C3CCC(=O)NC3=O)C4=O)c2)c1 ; O=C(COc1cccc2c1C(=O)N(C1CCC(=O)NC1=O)C2=O)NCCCCCCCCNC(=O)c1cccc(C#Cc2cc(NC(=O)Nc3ccnc(Cl)c3)ccn2)c1 ; O=C1CCC(N2C(=O)c3cccc(NCCCCCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCOCCOCCNC(=O)c4cccc(C#Cc5cc(NC(=O)Nc6ccnc(Cl)c6)ccn5)c4)c3C2=O)C(=O)N1]

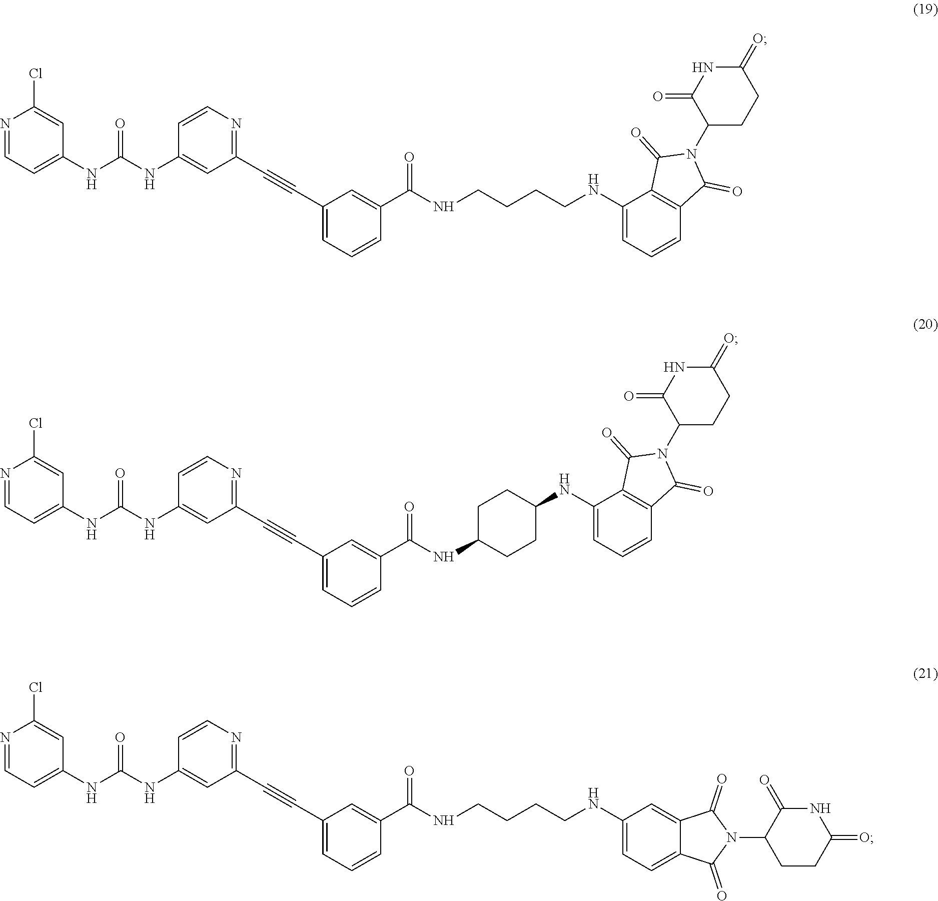 [SMILES: O=C1CCC(N2C(=O)c3cccc(NCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(N[C@H]4CC[C@@H](NC(=O)c5cccc(C#Cc6cc(NC(=O)Nc7ccnc(Cl)c7)ccn6)c5)CC4)c3C2=O)C(=O)N1 ; O=C1CCC(N2C(=O)c3ccc(NCCCCNC(=O)c4cccc(C#Cc5cc(NC(=O)Nc6ccnc(Cl)c6)ccn5)c4)cc3C2=O)C(=O)N1]

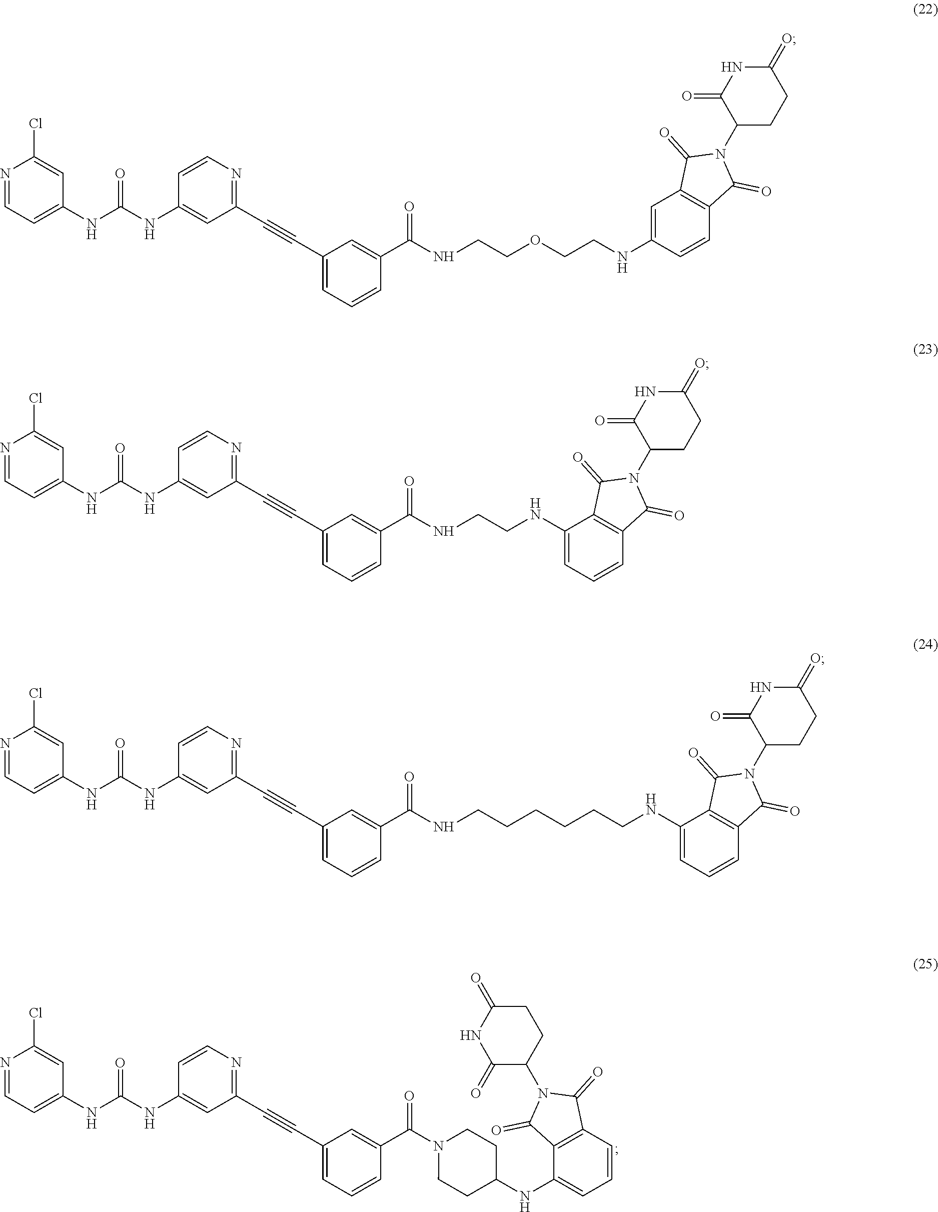 [SMILES: O=C1CCC(N2C(=O)c3ccc(NCCOCCNC(=O)c4cccc(C#Cc5cc(NC(=O)Nc6ccnc(Cl)c6)ccn5)c4)cc3C2=O)C(=O)N1 ; O=C1CCC(N2C(=O)c3cccc(NCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NC4CCN(C(=O)c5cccc(C#Cc6cc(NC(=O)Nc7ccnc(Cl)c7)ccn6)c5)CC4)c3C2=O)C(=O)N1]

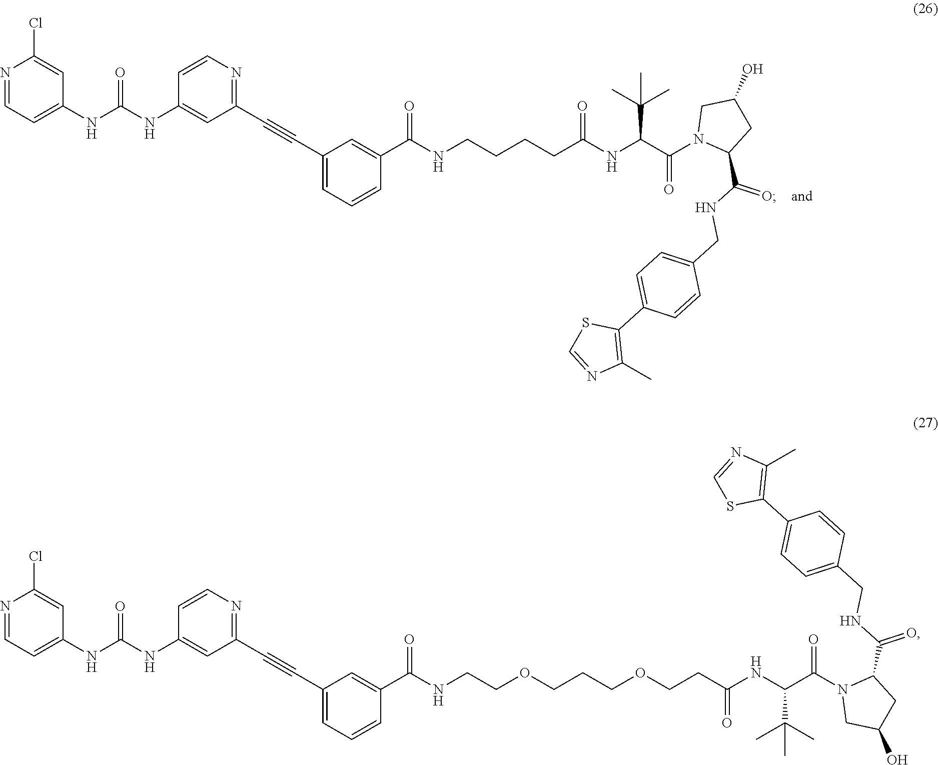 [SMILES: Cc1ncsc1-c1ccc(CNC(=O)[C@@H]2C[C@@H](O)CN2C(=O)[C@@H](NC(=O)CCCCNC(=O)c2cccc(C#Cc3cc(NC(=O)Nc4ccnc(Cl)c4)ccn3)c2)C(C)(C)C)cc1 ; "and" ; Cc1ncsc1-c1ccc(CNC(=O)[C@@H]2C[C@@H](O)CN2C(=O)[C@@H](NC(=O)CCOCCCOCCNC(=O)c2cccc(C#Cc3cc(NC(=O)Nc4ccnc(Cl)c4)ccn3)c2)C(C)(C)C)cc1]

or a pharmaceutically acceptable salt or stereoisomer thereof.

Bifunctional compounds of formula (I) may be in the form of a free acid or free base, or a pharmaceutically acceptable salt. A pharmaceutically acceptable salt of the compounds of this disclosure can be formed, for example, by reaction of an appropriate free base of a compound of the disclosure and an appropriate pharmaceutically acceptable acid in a suitable solvent under standard conditions well known in the art. See, for example, Gould, P. L., “Salt selection for basic drugs,” International Journal of Pharmaceutics, 33: 201-217 (1986); Bastin, R. J., et al., “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19, (1977).

As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn, and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate, or p-toluenesulfonate salts and the like. Certain compounds of the invention can form pharmaceutically acceptable salts with various organic bases such as lysine, arginine, guanidine, diethanolamine or metformin.

Bifunctional compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R—) or (S—) configurations of the compounds), mixtures of mirror image isomers (physical mixtures of the enantiomers, and racemates or racemic mixtures) of compounds, geometric (cis/trans or E/Z, R/S) isomers of compounds, and isomers of compounds with more than one chiral center that are not mirror images of one another (diastereoisomers). The chiral centers of the compounds may undergo epimerization in vivo; thus, for these compounds, administration of the compound in its (R—) form is considered equivalent to administration of the compound in its (S—) form. Accordingly, the compounds of the present disclosure may be made and used in the form of individual isomers and substantially free of other isomers, or in the form of a mixture of various isomers, e.g., racemic mixtures of stereoisomers.

In some embodiments, the bifunctional compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. As used herein, the term “compound” embraces isotopic derivatives. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

In addition, bifunctional compounds of formula (I) embrace N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

Another aspect of the present disclosure is directed to a method for making a bifunctional compound of formula (I) or a pharmaceutically acceptable salt or stereoisomer thereof. Broadly, the compounds or pharmaceutically acceptable salts or stereoisomers thereof, may be prepared by any process known to be applicable to the preparation of chemically related compounds. The compounds of the present disclosure will be better understood in connection with the synthetic schemes that described in various working examples that illustrate nonlimiting methods by which the compounds of the disclosure may be prepared.

### Pharmaceutical Compositions

Another aspect of the present disclosure is directed to a pharmaceutical composition that includes a therapeutically effective amount of a bifunctional compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bifunctional compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly, bifunctional compounds of the present disclosure may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs), semi-solid compositions (e.g., gels, suspensions and creams), and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of the present disclosure may be formulated in a hard or soft gelatin capsule. Representative excipients that may be used include pregelatinized starch, magnesium stearate, mannitol, sodium stearyl fumarate, lactose anhydrous, microcrystalline cellulose, and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipient such as wetting agents, suspending agents, coloring, sweetening, flavoring, and perfuming agents.

Injectable preparations may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, bifunctional compounds of formula (I) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters), and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The bifunctional compounds may be formulated for buccal or sublingual administration, examples of which include tablets, lozenges and gels.

The bifunctional compounds may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula (I) may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a bifunctional compound of formula (I) or a pharmaceutically acceptable salt or a stereoisomer thereof, or a composition including a bifunctional compound of formula (I) or a pharmaceutically acceptable salt or a stereoisomer thereof, effective in producing the desired therapeutic response in a particular patient suffering from a disease or disorder characterized or mediated by aberrant activity of SMARCA4 or SMARCA2 and SMARCA4. The term “therapeutically effective amount” thus includes the amount of a bifunctional compound of the disclosure or a pharmaceutically acceptable salt or a stereoisomer thereof, that when administered, induces a positive modification in the disease or disorder to be treated, or is sufficient to inhibit or even prevent development or progression of the disease or disorder, or alleviate to some extent, one or more of the symptoms of the disease or disorder being treated in a subject, or which simply kills or inhibits the growth of diseased (e.g., cancer) cells, or reduces the amount of SMARCA4 or SMARCA2 and SMARCA4 in diseased cells.

The total daily dosage of the bifunctional compounds and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the bifunctional compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001). Bifunctional compounds of formula (I) and their pharmaceutically acceptable salts and stereoisomers may be effective over a wide dosage range. In some embodiments, the total daily dosage (e.g., for adult humans) may range from about 0.001 to about 1600 mg, from 0.01 to about 1600 mg, from 0.01 to about 500 mg, from about 0.01 to about 100 mg, from about 0.5 to about 100 mg, from 1 to about 100-400 mg per day, from about 1 to about 50 mg per day, and from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of a bifunctional compound (e.g., 1, 2, 2.5, 3, 4, 5, 10, 15, 20, 25, 50, 100, 150, and 200 mg). In some embodiments, individual dosages may be formulated to contain the desired dosage amount depending upon the number of times the compound is administered per day. Methods of Use

In some aspects, the present disclosure is directed to methods of treating diseases or disorders involving aberrant (e.g., dysfunctional or dysregulated) activity of SMARCA4 or SMARCA2 and SMARCA4, that entails administration of a therapeutically effective amount of a bifunctional compound formula (I) or a pharmaceutically acceptable salt or stereoisomer thereof, to a subject in need thereof.

The diseases or disorders are characterized or mediated by aberrant activity SMARCA4 or SMARCA2 and SMARCA4 (e.g., elevated levels of SMARCA4 or SMARCA2 and SMARCA4 or otherwise functionally abnormal SMARCA4 or SMARCA2 and SMARCA4 relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject is suffering from the disease or disorder. Thus, subjects suffering from, and suspected of suffering from, a specific disease or disorder are not necessarily two distinct groups.

In some embodiments, bifunctional compounds of formula (I) may be useful in the treatment of cell proliferative diseases and disorders (e.g., cancer or benign neoplasms). As used herein, the term “cell proliferative disease or disorder” refers to the conditions characterized by deregulated or abnormal cell growth, or both, including noncancerous conditions such as neoplasms, precancerous conditions, benign tumors, and cancer.

In other embodiments, the methods are directed to treating subjects having cancer. Broadly, the bifunctional compounds of the present disclosure may be effective in the treatment of carcinomas (solid tumors including both primary and metastatic tumors), sarcomas, melanomas, and hematological cancers (cancers affecting blood including lymphocytes, bone marrow and/or lymph nodes) such as leukemia, lymphoma and multiple myeloma. Adult tumors/cancers and pediatric tumors/cancers are included. The cancers may be vascularized, or not yet substantially vascularized, or non-vascularized tumors.

Representative examples of cancers include adrenocortical carcinoma, AIDS-related cancers (e.g., Kaposi's and AIDS-related lymphoma), appendix cancer, childhood cancers (e.g., childhood cerebellar astrocytoma, childhood cerebral astrocytoma), basal cell carcinoma, skin cancer (non-melanoma), biliary cancer, extrahepatic bile duct cancer, intrahepatic bile duct cancer, bladder cancer, urinary bladder cancer, brain cancer (e.g., gliomas and glioblastomas such as brain stem glioma, gestational trophoblastic tumor glioma, cerebellar astrocytoma, cerebral astrocytoma/malignant glioma, ependymoma, medulloblastoma, supratentorial primitive neuroectodeimal tumors, visual pathway and hypothalamic glioma), breast cancer, bronchial adenomas/carcinoids, carcinoid tumor, nervous system cancer (e.g., central nervous system cancer, central nervous system lymphoma), cervical cancer, chronic myeloproliferative disorders, colorectal cancer (e.g., colon cancer, rectal cancer), lymphoid neoplasm, mycosis fungoids, Sezary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastrointestinal cancer (e.g., stomach cancer, small intestine cancer, gastrointestinal carcinoid tumor, gastrointestinal stromal tumor (GIST)), cholangiocarcinoma, germ cell tumor, ovarian germ cell tumor, head and neck cancer, neuroendocrine tumors, Hodgkin's lymphoma, Ann Arbor stage III and stage IV childhood Non-Hodgkin's lymphoma, ROS1-positive refractory Non-Hodgkin's lymphoma, leukemia, lymphoma, multiple myeloma, hypopharyngeal cancer, intraocular melanoma, ocular cancer, islet cell tumors (endocrine pancreas), renal cancer (e.g., Wilm's Tumor, renal cell carcinoma), liver cancer, lung cancer (e.g., non-small cell lung cancer and small cell lung cancer), ALK-positive anaplastic large cell lymphoma, ALK-positive advanced malignant solid neoplasm, Waldenstrom's macroglobulinema, melanoma, intraocular (eye) melanoma, merkel cell carcinoma, mesothelioma, metastatic squamous neck cancer with occult primary, multiple endocrine neoplasia (MEN), myelodysplastic syndromes, myelodysplastic/myeloproliferative diseases, nasopharyngeal cancer, neuroblastoma, oral cancer (e.g., mouth cancer, lip cancer, oral cavity cancer, tongue cancer, oropharyngeal cancer, throat cancer, laryngeal cancer), ovarian cancer (e.g., ovarian epithelial cancer, ovarian germ cell tumor, ovarian low malignant potential tumor), pancreatic cancer, islet cell pancreatic cancer, paranasal sinus and nasal cavity cancer, parathyroid cancer, penile cancer, pharyngeal cancer, pheochromocytoma, pineoblastoma, metastatic anaplastic thyroid cancer, undifferentiated thyroid cancer, papillary thyroid cancer, pituitary tumor, plasma cell neoplasm/multiple myeloma, pleuropulmonary blastoma, prostate cancer, retinoblastoma, rhabdomyosarcoma, salivary gland cancer, uterine cancer (e.g., endometrial uterine cancer, uterine sarcoma, uterine corpus cancer), squamous cell carcinoma, testicular cancer, thymoma, thymic carcinoma, thyroid cancer, juvenile xanthogranuloma, transitional cell cancer of the renal pelvis and ureter and other urinary organs, urethral cancer, gestational trophoblastic tumor, vaginal cancer, vulvar cancer, hepatoblastoma, rhabdoid tumor, and Wilms tumor.

Sarcomas that may be treatable with the bifunctional compounds of the present disclosure include both soft tissue and bone cancers alike, representative examples of which include osteosarcoma or osteogenic sarcoma (bone) (e.g., Ewing's sarcoma), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), mesenchymous or mixed mesodermal tumor (mixed connective tissue types), and histiocytic sarcoma (immune cancer).

In some embodiments, methods of the present disclosure entail treatment of subjects having cell proliferative diseases or disorders of the hematological system, liver, brain, lung, colon, pancreas, prostate, ovary, breast, skin, and endometrium.

As used herein, “cell proliferative diseases or disorders of the hematological system” include lymphoma, leukemia, myeloid neoplasms, mast cell neoplasms, myelodysplasia, benign monoclonal gammopathy, lymphomatoid papulosis, polycythemia vera, agnogenic myeloid metaplasia, and essential thrombocythemia. Representative examples of hematologic cancers may thus include multiple myeloma, lymphoma (including T-cell lymphoma, Hodgkin's lymphoma, non-Hodgkin's lymphoma (diffuse large B-cell lymphoma (DLBCL), follicular lymphoma (FL), mantle cell lymphoma (MCL) and ALK+ anaplastic large cell lymphoma (e.g., B-cell non-Hodgkin's lymphoma selected from diffuse large B-cell lymphoma (e.g., germinal center B-cell-like diffuse large B-cell lymphoma or activated B-cell-like diffuse large B-cell lymphoma), Burkitt's lymphoma/leukemia, mantle cell lymphoma, mediastinal (thymic) large B-cell lymphoma, follicular lymphoma, marginal zone lymphoma, lymphoplasmacytic lymphoma/Waldenstrom macroglobulinemia, metastatic pancreatic adenocarcinoma, refractory B-cell non-Hodgkin's lymphoma, and relapsed B-cell non-Hodgkin's lymphoma, childhood lymphomas, and lymphomas of lymphocytic and cutaneous origin, e.g., small lymphocytic lymphoma, leukemia, including childhood leukemia, hairy-cell leukemia, acute lymphocytic leukemia, acute myelocytic leukemia, acute myeloid leukemia (e.g., acute monocytic leukemia), chronic lymphocytic leukemia, small lymphocytic leukemia, chronic myelocytic leukemia, chronic myelogenous leukemia, and mast cell leukemia, myeloid neoplasms and mast cell neoplasms.

As used herein, “cell proliferative diseases or disorders of the liver” include all forms of cell proliferative disorders affecting the liver. Cell proliferative disorders of the liver may include liver cancer (e.g., hepatocellular carcinoma, intrahepatic cholangiocarcinoma and hepatoblastoma), a precancer or precancerous condition of the liver, benign growths or lesions of the liver, and malignant growths or lesions of the liver, and metastatic lesions in tissue and organs in the body other than the liver. Cell proliferative disorders of the liver may include hyperplasia, metaplasia, and dysplasia of the liver.

As used herein, “cell proliferative diseases or disorders of the brain” include all forms of cell proliferative disorders affecting the brain. Cell proliferative disorders of the brain may include brain cancer (e.g., gliomas, glioblastomas, meningiomas, pituitary adenomas, vestibular schwannomas, and primitive neuroectodermal tumors (medulloblastomas)), a precancer or precancerous condition of the brain, benign growths or lesions of the brain, and malignant growths or lesions of the brain, and metastatic lesions in tissue and organs in the body other than the brain. Cell proliferative disorders of the brain may include hyperplasia, metaplasia, and dysplasia of the brain.

As used herein, “cell proliferative diseases or disorders of the lung” include all forms of cell proliferative disorders affecting lung cells. Cell proliferative disorders of the lung include lung cancer, precancer and precancerous conditions of the lung, benign growths or lesions of the lung, hyperplasia, metaplasia, and dysplasia of the lung, and metastatic lesions in the tissue and organs in the body other than the lung. Lung cancer includes all forms of cancer of the lung, e.g., malignant lung neoplasms, carcinoma in situ, typical carcinoid tumors, and atypical carcinoid tumors. Lung cancer includes small cell lung cancer (“SLCL”), non-small cell lung cancer (“NSCLC”), adenocarcinoma, small cell carcinoma, large cell carcinoma, squamous cell carcinoma, and mesothelioma. Lung cancer can include “scar carcinoma”, bronchioveolar carcinoma, giant cell carcinoma, spindle cell carcinoma, and large cell neuroendocrine carcinoma. Lung cancer also includes lung neoplasms having histologic and ultrastructural heterogeneity (e.g., mixed cell types). In some embodiments, a compound of the present disclosure may be used to treat non-metastatic or metastatic lung cancer (e.g., NSCLC, ALK-positive NSCLC, NSCLC harboring ROS1 rearrangement, lung adenocarcinoma, and squamous cell lung carcinoma).

As used herein, “cell proliferative diseases or disorders of the colon” include all forms of cell proliferative disorders affecting colon cells, including colon cancer, a precancer or precancerous conditions of the colon, adenomatous polyps of the colon and metachronous lesions of the colon. Colon cancer includes sporadic and hereditary colon cancer, malignant colon neoplasms, carcinoma in situ, typical carcinoid tumors, and atypical carcinoid tumors, adenocarcinoma, squamous cell carcinoma, and squamous cell carcinoma. Colon cancer can be associated with a hereditary syndrome such as hereditary nonpolyposis colorectal cancer, familiar adenomatous polyposis, MYH associated polyposis, Gardner's syndrome, Peutz-Jeghers syndrome, Turcot's syndrome and juvenile polyposis. Cell proliferative disorders of the colon may also be characterized by hyperplasia, metaplasia, or dysplasia of the colon.

As used herein, “cell proliferative diseases or disorders of the pancreas” include all forms of cell proliferative disorders affecting pancreatic cells. Cell proliferative disorders of the pancreas may include pancreatic cancer, a precancer or precancerous condition of the pancreas, hyperplasia of the pancreas, dysplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas, including ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma, and pancreatic neoplasms having histologic and ultrastructural heterogeneity (e.g., mixed cell).

As used herein, “cell proliferative diseases or disorders of the prostate” include all forms of cell proliferative disorders affecting the prostate. Cell proliferative disorders of the prostate may include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate may include hyperplasia, metaplasia, and dysplasia of the prostate.

As used herein, “cell proliferative diseases or disorders of the ovary” include all forms of cell proliferative disorders affecting cells of the ovary. Cell proliferative disorders of the ovary may include a precancer or precancerous condition of the ovary, benign growths or lesions of the ovary, ovarian cancer, and metastatic lesions in tissue and organs in the body other than the ovary. Cell proliferative disorders of the ovary may include hyperplasia, metaplasia, and dysplasia of the ovary.

As used herein, “cell proliferative diseases or disorders of the breast” include all forms of cell proliferative disorders affecting breast cells. Cell proliferative disorders of the breast may include breast cancer, a precancer or precancerous condition of the breast, benign growths or lesions of the breast, and metastatic lesions in tissue and organs in the body other than the breast. Cell proliferative disorders of the breast may include hyperplasia, metaplasia, and dysplasia of the breast.

As used herein, “cell proliferative diseases or disorders of the skin” include all forms of cell proliferative disorders affecting skin cells. Cell proliferative disorders of the skin may include a precancer or precancerous condition of the skin, benign growths or lesions of the skin, melanoma, malignant melanoma or other malignant growths or lesions of the skin, and metastatic lesions in tissue and organs in the body other than the skin. Cell proliferative disorders of the skin may include hyperplasia, metaplasia, and dysplasia of the skin.

As used herein, “cell proliferative diseases or disorders of the endometrium” include all forms of cell proliferative disorders affecting cells of the endometrium. Cell proliferative disorders of the endometrium may include a precancer or precancerous condition of the endometrium, benign growths or lesions of the endometrium, endometrial cancer, and metastatic lesions in tissue and organs in the body other than the endometrium. Cell proliferative disorders of the endometrium may include hyperplasia, metaplasia, and dysplasia of the endometrium.

In some embodiments, the cancer is selected from synovial sarcoma, lung cancer, ovarian cancer, brain cancer, kidney cancer, leukemia, non-small cell lung cancer, Burkitt's Lymphoma, childhood medulloblastoma, pancreatic adenocarcinoma, ovarian clear cell carcinoma, renal cell carcinoma, endometrial carcinoma, and melanoma.

In some aspects, the present disclosure is directed to methods of treating a diffuse midline glioma harboring a H3K27M mutation, comprising administering to a subject in need thereof a therapeutically effective amount of a bifunctional compound of formula (I) or pharmaceutically acceptable salt or stereoisomer thereof. In another aspect, the present disclosure is directed to methods of treating a diffuse midline glioma harboring a H3K27M mutation, comprising administering to a subject in need thereof a therapeutically effective amount of a bifunctional compound of any of formulas I′, I″ and II″.

In some embodiments, the method of treating a diffuse midline glioma harboring a H3K27M mutation comprises administering to a subject in need thereof a therapeutically effective amount of a bifunctional compound of formula I′:

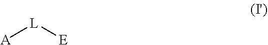

or a pharmaceutically acceptable salt thereof, wherein A is

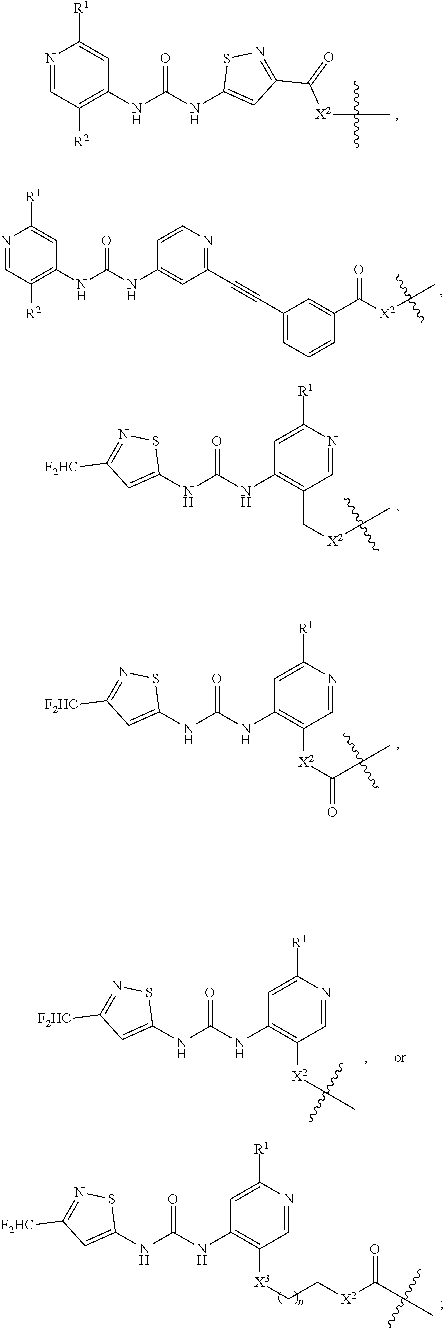

### E is

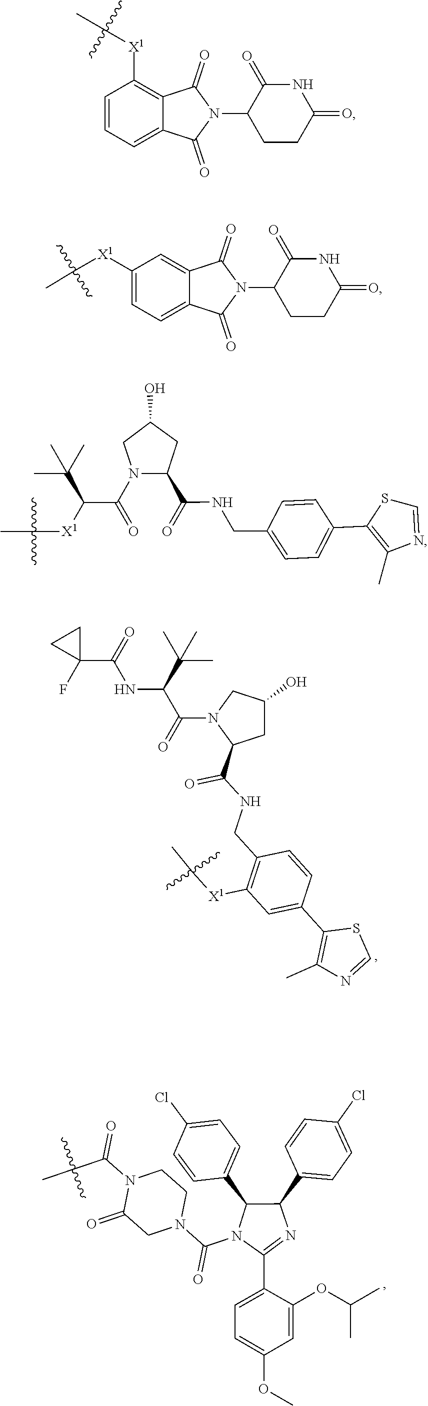

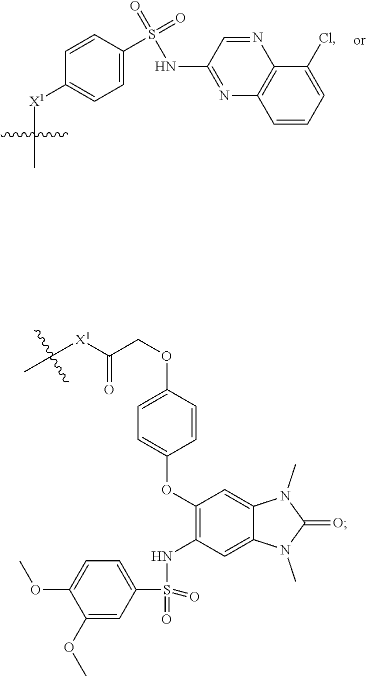

X¹ and X² are each independently selected from O, NR³, and S; or X¹ or X² combine with L to form a heterocyclyl or heteroaryl; or both of X¹ and X² combine with L to form a heterocyclyl or heteroaryl;
X³ is O, NR³, or S;
L comprises an alkylene, alkenylene, alkynylene, cycloalkylene, heterocyclylene, arylene, or heteroarylene chain, or a combination thereof, comprising 1 to 35 carbon atoms, for example, 1 to 35 —CH₂— moieties, optionally wherein: if L comprises an alkylene comprising 1 to 35 —CH₂— moieties, then at least one, but no more than ten, —CH₂— moieties of L are independently replaced with a moiety selected from —C(═O)—, —C(═O)—NR⁴—, —NR⁴—C(═O)—, —C(═O)—O—, —O—C(═O)—, —NR⁴—C(═O)—NR³—, —O—C(═O)—NR⁴—, —NR⁴—C(═O)—O—, —O—, —S—, and —NR⁴—, provided that the number of —CH2— moieties of L is larger than the collective number of —C(═O)—, —C(═O)—NR⁴—, —NR⁴—C(═O)—, —C(═O)—O—, —O—C(═O)—, —NR⁴—C(═O)—NR³—, O—C(═O)—NR⁴—, —NR⁴—C(═O)—O—, —O—, —S—, and —NR— moieties of L, and provided there is at least one —CH2— between each —C(═O)—, —C(═O)—NR⁴—, —NR⁴—C(═O)—, —C(═O)—O—, —O—C(═O)—, —NR⁴—C(═O)—NR³—, —O—C(═O)—NR⁴—, —NR⁴—C(═O)—O—, —O—, —S—, and —NR⁴— moiety of L;
R¹ and R² are each independently selected from H, alkyl, halo, hydroxyl, hydroxalkyl, carboxyl, acyl, ester, thioester, alkoxy, phosphoryl, amino, amido, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl;
R³ and R⁴ are each independently selected from H and alkyl; and
n is 1-5, preferably 1.

In some embodiments, the bifunctional compound of formula I′ is selected from the following:

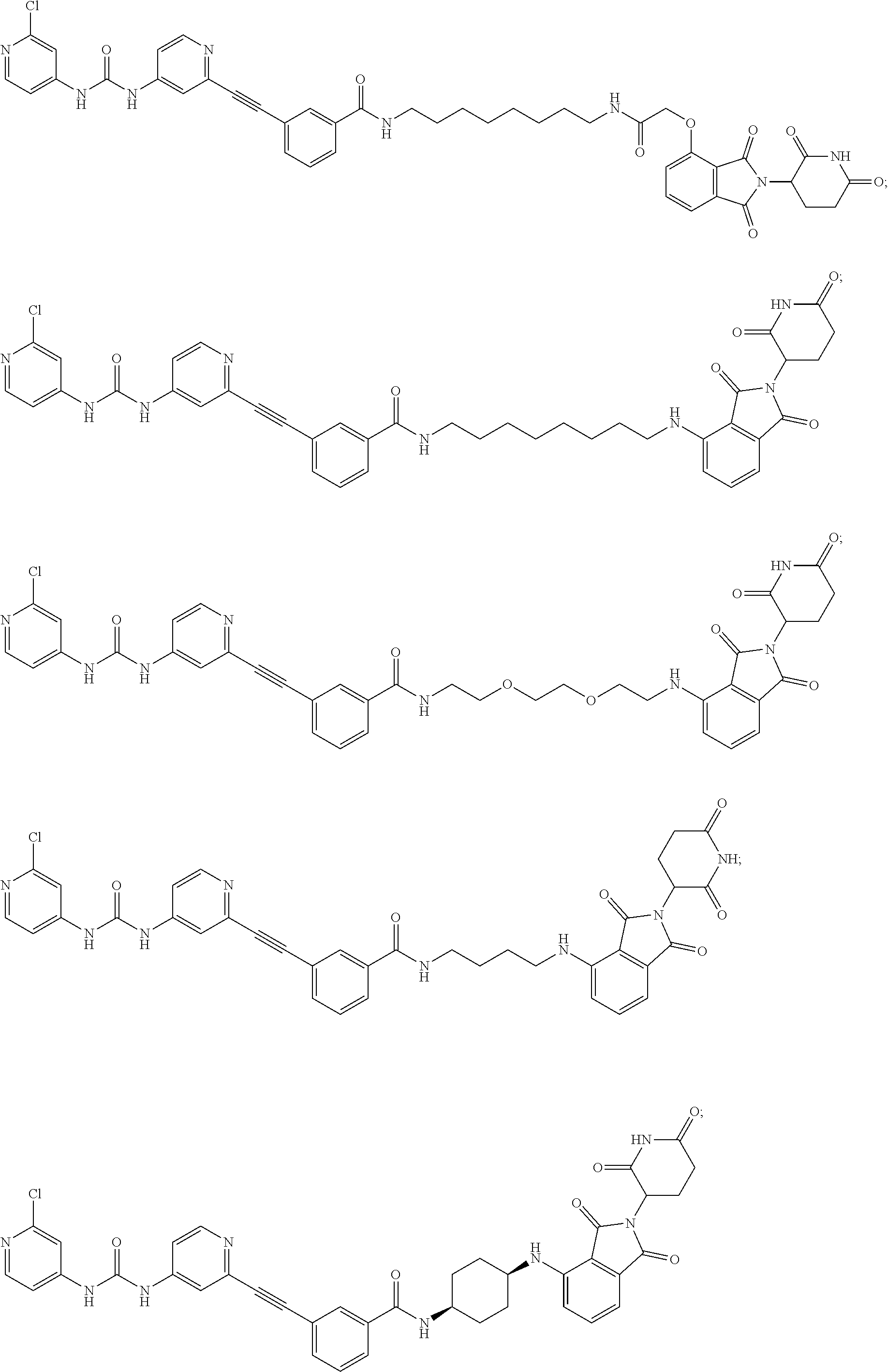 [SMILES: O=C(COc1cccc2c1C(=O)N(C1CCC(=O)NC1=O)C2=O)NCCCCCCCCNC(=O)c1cccc(C#Cc2cc(NC(=O)Nc3ccnc(Cl)c3)ccn2)c1 ; O=C1CCC(N2C(=O)c3cccc(NCCCCCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCOCCOCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(N[C@H]4CC[C@@H](NC(=O)c5cccc(C#Cc6cc(NC(=O)Nc7ccnc(Cl)c7)ccn6)c5)CC4)c3C2=O)C(=O)N1]

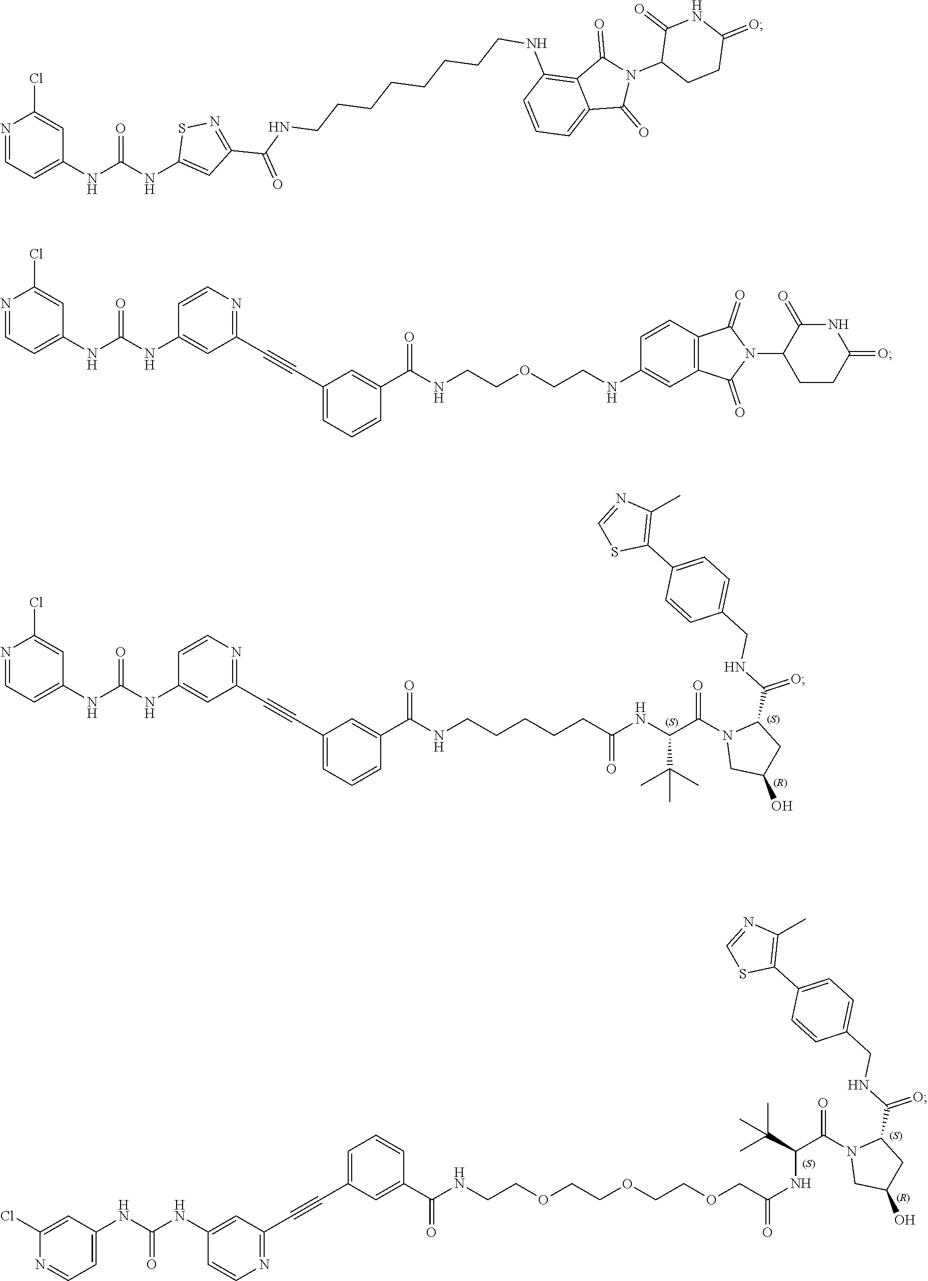

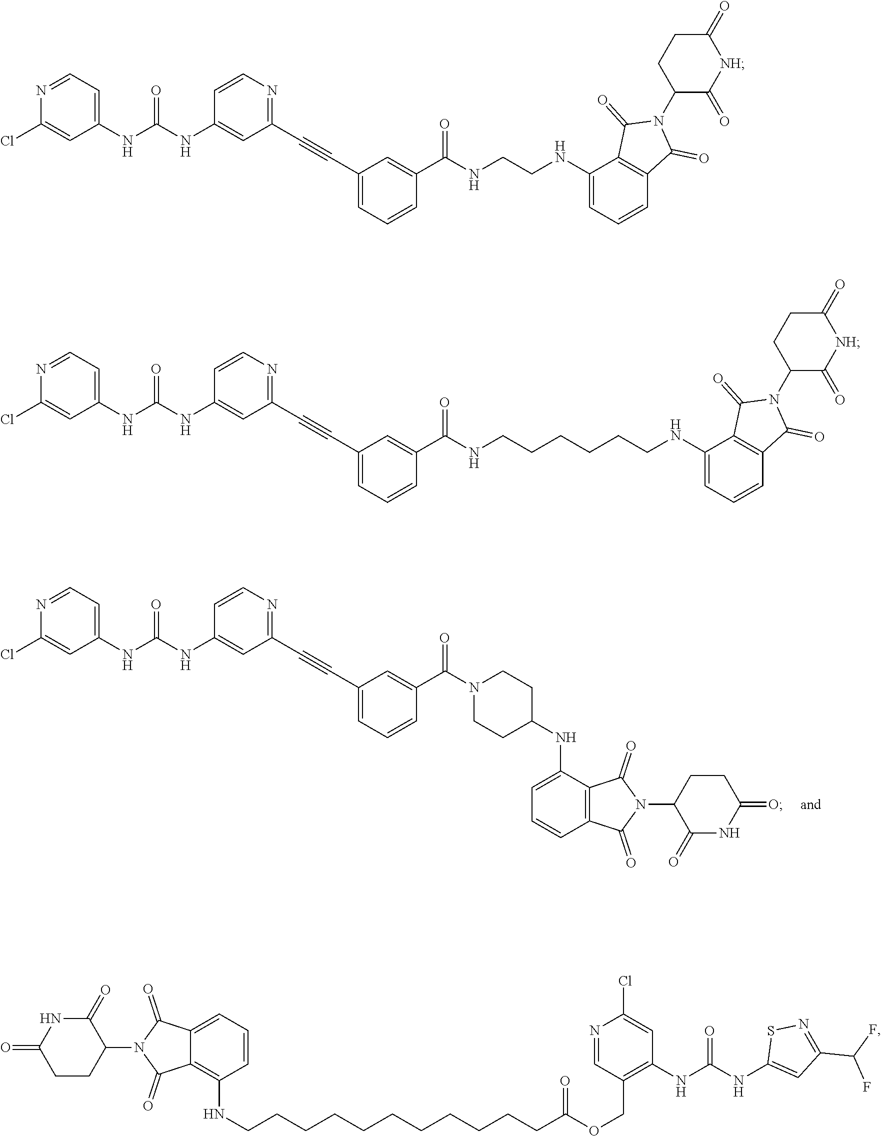 [SMILES: O=C1CCC(N2C(=O)c3cccc(NCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NC4CCN(C(=O)c5cccc(C#Cc6cc(NC(=O)Nc7ccnc(Cl)c7)ccn6)c5)CC4)c3C2=O)C(=O)N1 ; O=C1CCC(N2C(=O)c3cccc(NCCCCCCCCCCCC(=O)OCc4cnc(Cl)cc4NC(=O)Nc4cc(C(F)F)ns4)c3C2=O)C(=O)N1]

or a pharmaceutically acceptable salt or stereoisomer thereof.

Compounds of formula I′ are described in PCT/US2021/017626, the contents of which are incorporated herein by reference.

In some embodiments, the method of treating a diffuse midline glioma harboring a H3K27M mutation comprises administering to a subject in need thereof a therapeutically effective amount of a bifunctional compound of formula I″ or II″:

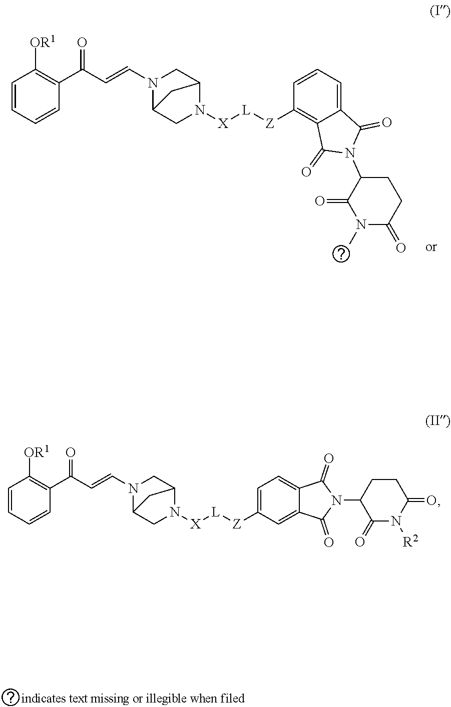

or a pharmaceutically acceptable salt thereof, wherein
X is a bond, aryl, or heteroaryl;
L is an alkylene, alkenylene, or alkynylene chain comprising 1 to 35 carbon atoms, for example, 1 to 35 —CH₂— moieties,
   optionally wherein:
   at least one, but no more than ten, —CH2— moieties of L are independently replaced with a moiety selected from —C(═O)—, —C(═O)—NR³—, —NR³—C(═O)—, —C(═O)—, —O—C(═O)—, —NR³—C(═O)—NR³—, —O—C(═O)—NR³—, —NR³—C(═O)—O—, —O—, —S—, and —NR³, provided the number of —CH₂— moieties of L is larger than the collective number of —C(═O)—, —C(═O)—NR³—, —NR³—C(═O)—, —C(═O)—O—, —O—C(═O)—, —NR³—C(═O)—NR³—, —O—C(═O)—NR³—, —NR³—C(═O)—O—, —O—, —S—, and —NR³— moieties of L, and
   provided there is at least one —CH₂— between each —C(═O)—, —C(═O)—NR³—, —NR³—C(═O)—, —C(═O)—O—, —O—C(═O)—, —NR³—C(═O)—NR³, —O—C(═O)—NR³—, —NR³—C(═O)—O—, —O—, —S— and —NR³— moiety of L;
Z-L is —CH₂-L, —O—CH2-L, or —NR³—CH₂-L; and
R¹, R², and R³ are each independently selected from H4 and alkyl.

Therefore, in some embodiments, the bifunctional compound is selected from the group consisting of:

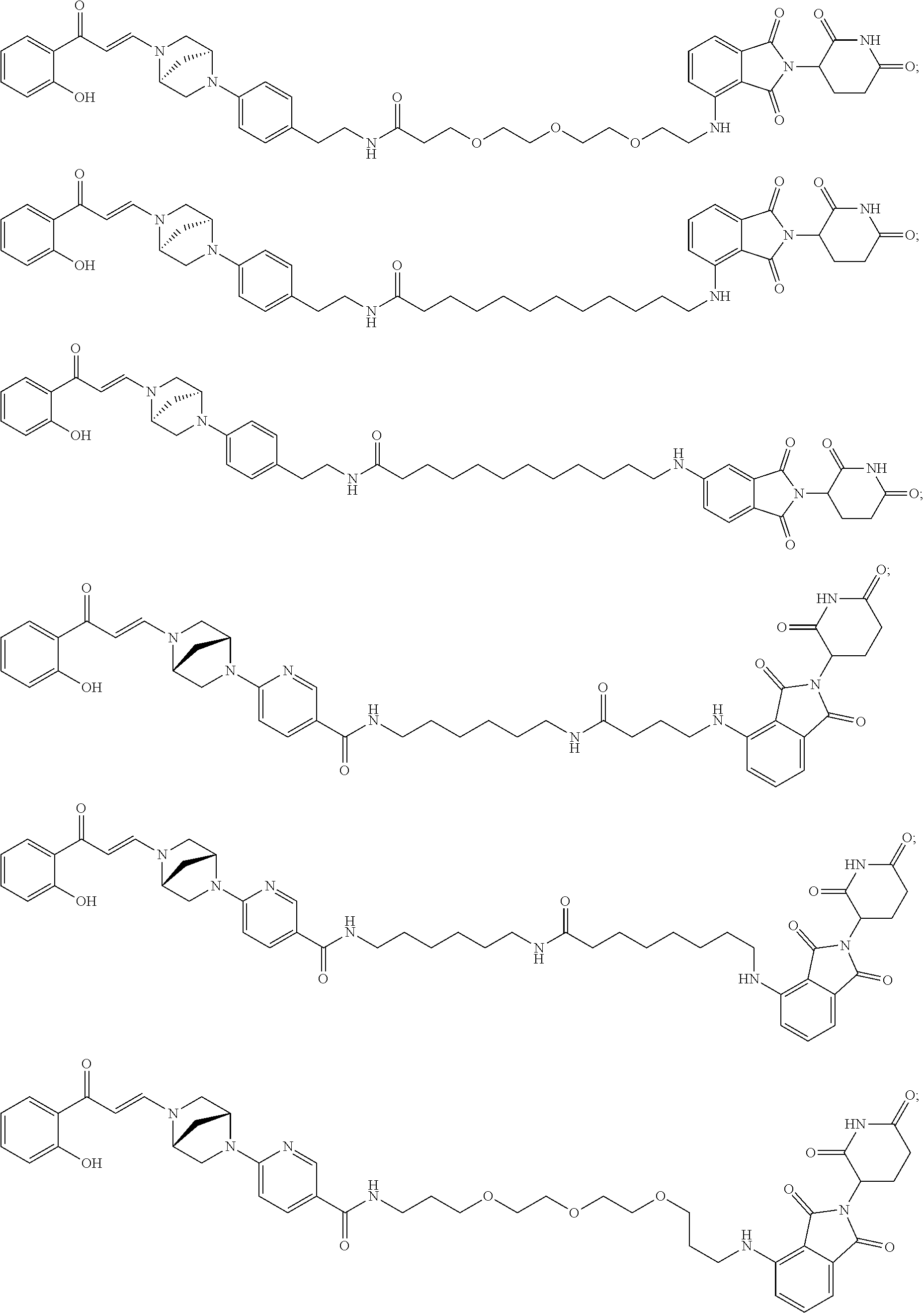

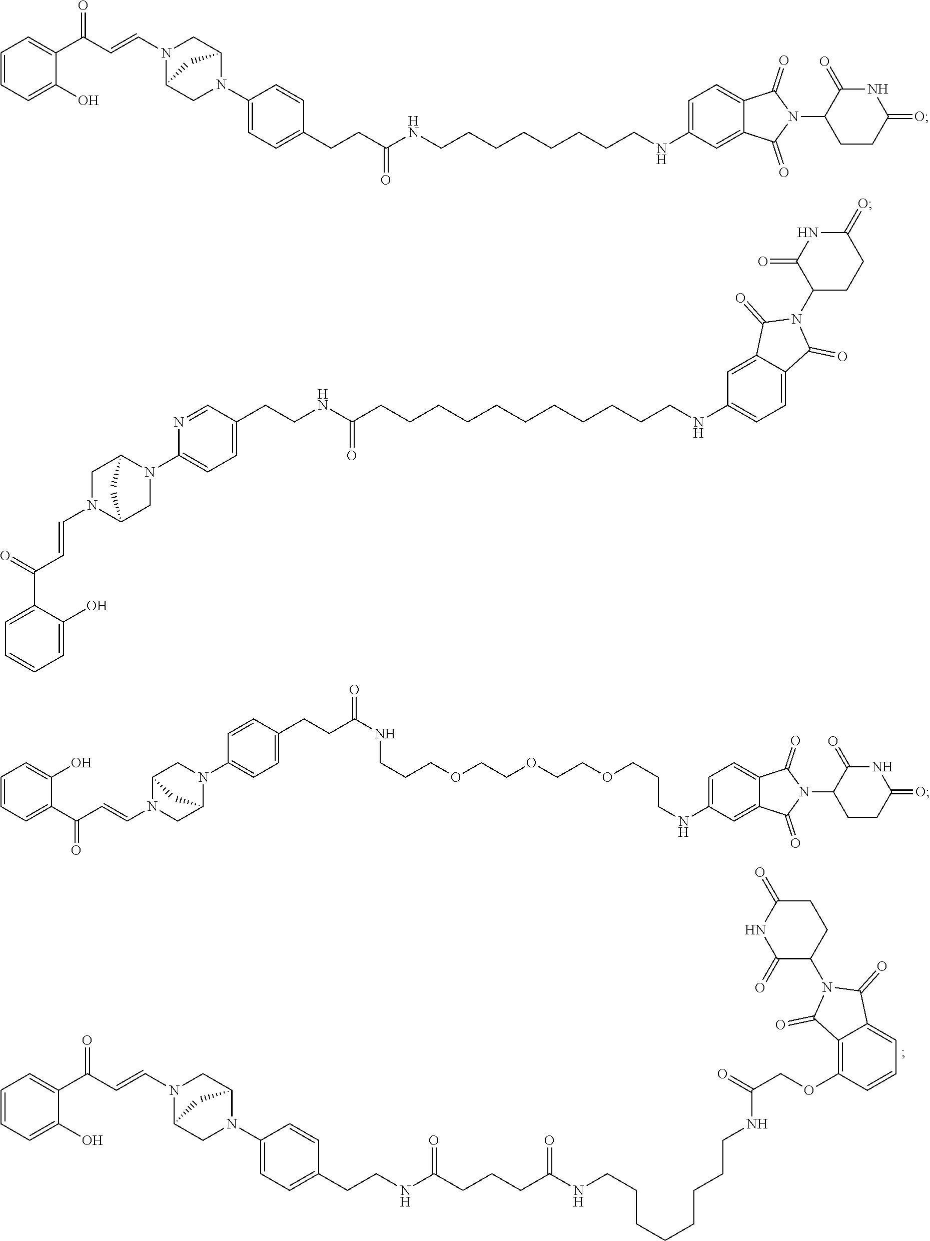

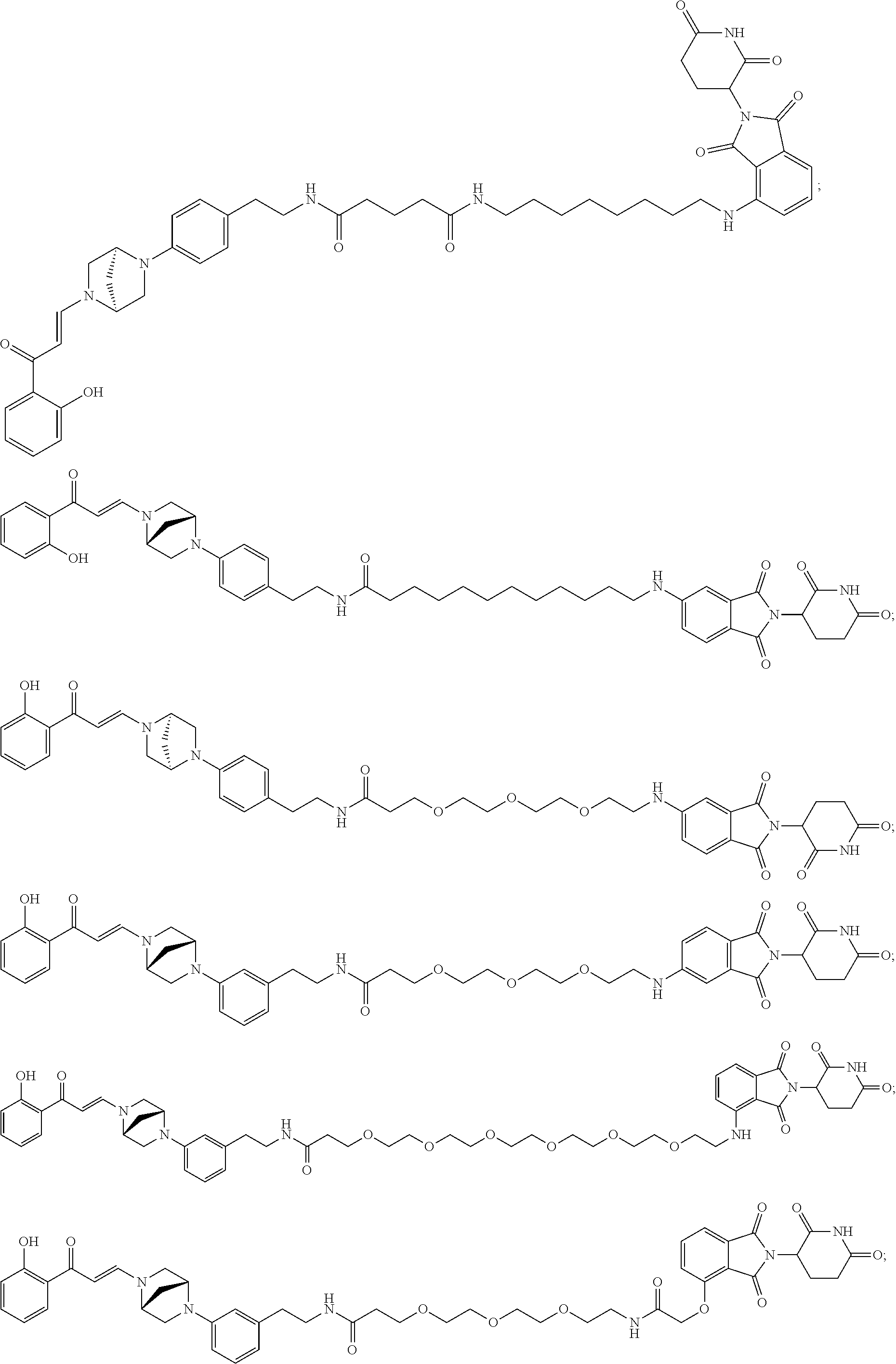

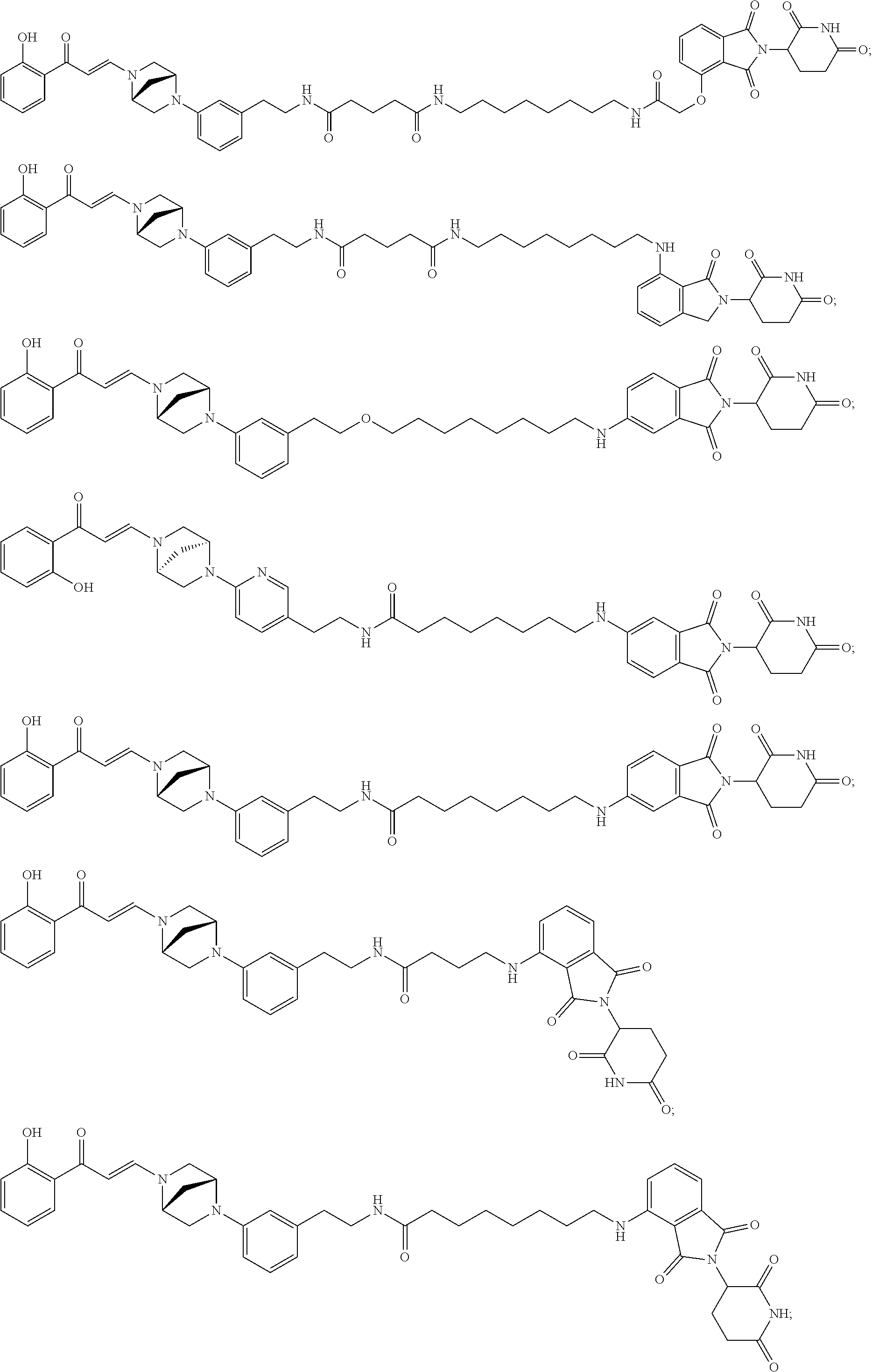

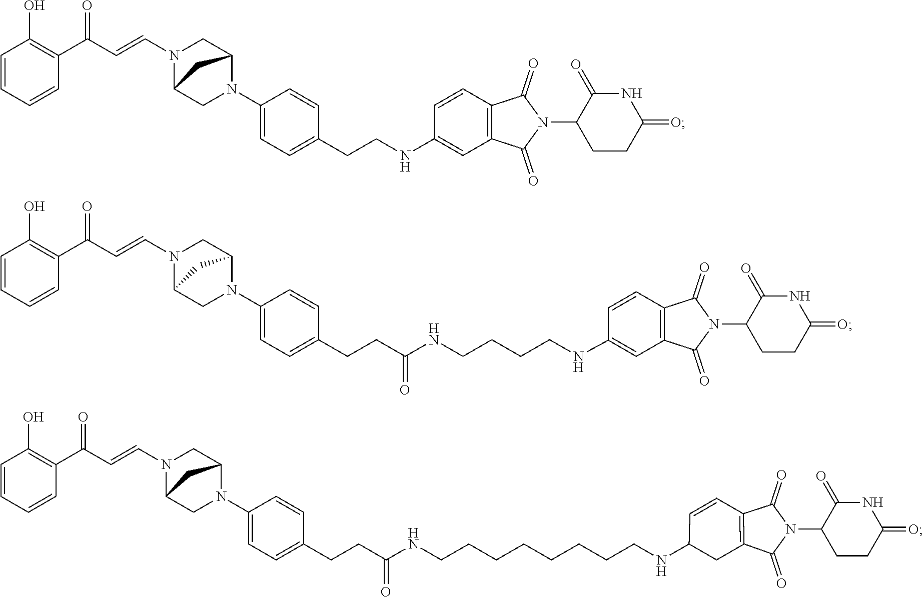 [SMILES: O=C1CCC(N2C(=O)c3ccc(NCCc4ccc(N5C[C@H]6C[C@@H]5CN6/C=C/C(=O)c5ccccc5O)cc4)cc3C2=O)C(=O)N1 ; O=C(CCc1ccc(N2C[C@@H]3C[C@H]2CN3/C=C/C(=O)c2ccccc2O)cc1)NCCCCNc1ccc2c(c1)C(=O)N(C1CCC(=O)NC1=O)C2=O ; O=C(CCc1ccc(N2C[C@H]3C[C@@H]2CN3/C=C/C(=O)c2ccccc2O)cc1)NCCCCCCCCNC1C=CC2=C(C1)C(=O)N(C1CCC(=O)NC1=O)C2=O]

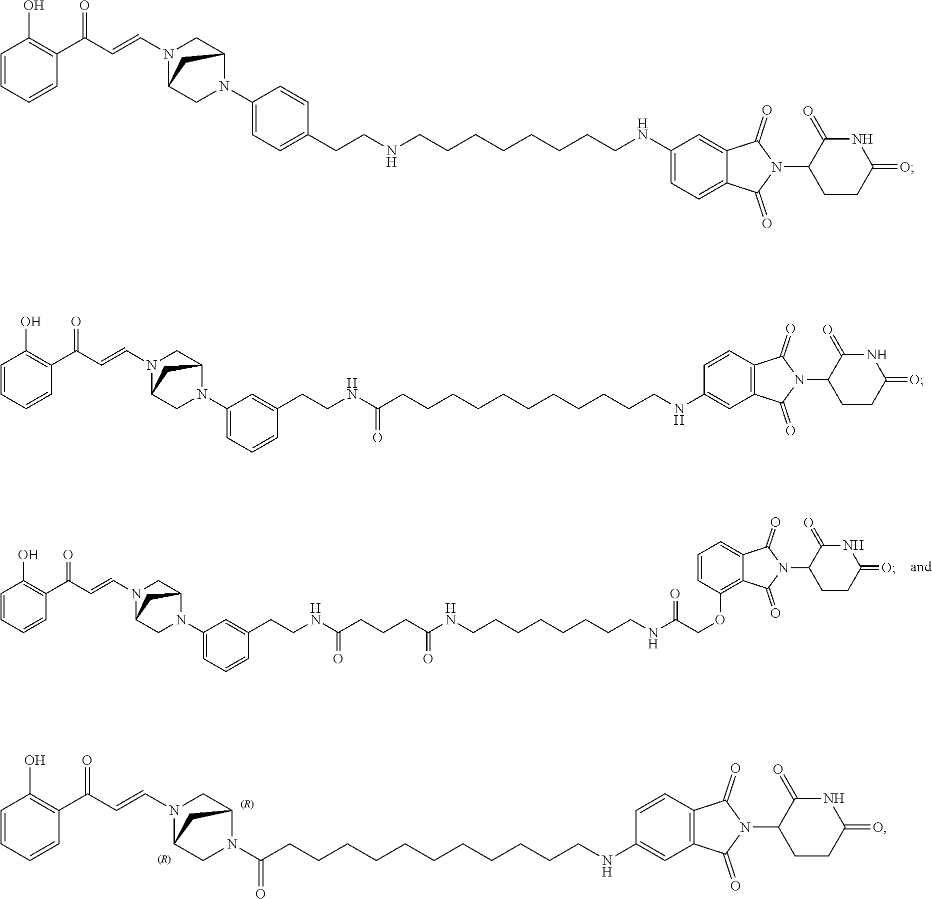

or a pharmaceutically acceptable salt or stereoisomer thereof.

Compounds of formula I″ and II″ are described in International Patent Application No PCT/US2020/039638, the contents of which are incorporated herein by reference in their entirety.

For purposes of treating a diffuse midline glioma harboring a H3K27M mutation, compounds of formulas I′, I″, and II″ may be formulated and administered consistent with the teachings herein in connection with compounds of formula I.

In some embodiments, the diffuse midline glioma harboring the H3K27M mutation is diffuse intrinsic pontine glioma (DIPG).

The bifunctional compounds may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became intolerant to the particular treatment. Therapy may also be given as an adjuvant treatment, i.e., to prevent reoccurrence of cancer in patients with no currently detectable disease or after surgical removal of a tumor. Thus, in some embodiments, the bifunctional compounds may be administered to a patient who has received another therapy, such as chemotherapy, radioimmunotherapy, surgical therapy, immunotherapy, radiation therapy, targeted therapy or any combination thereof.

The methods of the present disclosure may entail administration of bifunctional compounds or pharmaceutical compositions thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days) followed by a 7-day “off” period. In other embodiments, the bifunctional compound may be dosed twice a day (BID) over the course of two and a half days (for a total of 5 doses) or once a day (QD) over the course of two days (for a total of 2 doses). In other embodiments, the bifunctional compound may be dosed once a day (QD) over the course of five days.

### Combination Therapy

The bifunctional compounds and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a bifunctional compound in combination with one or more additional therapeutics or pharmaceutical agents known for use in treating the disease or condition (e.g., cancer). The dosage of the additional anticancer therapeutic may be the same or even lower than known or recommended doses. See, Hardman et al., eds., Goodman & Gilman's The Pharmacological Basis Of Therapeutics, 10th ed., McGraw-Hill, New York, 2001; Physician's Desk Reference 60th ed., 2006. For example, anti-cancer agents that may be suitable for use in combination with the bifunctional compounds are known in the art. See, e.g., U.S. Pat. No. 9,101,622 (Section 5.2 thereof) and U.S. Pat. No. 9,345,705 B2 (Columns 12-18 thereof). Representative examples of additional active agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bifunctional antibodies) and CAR-T therapy.

In some embodiments, the bifunctional compound and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

In some embodiments involving cancer treatment, the bifunctional compound and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the additional therapeutic or pharmaceutical agent is a B Lymphoma Mo-MLV Insertion Region 1 Homolog (BMI1) inhibitor. In some embodiments, the BMI1 inhibitor is PTC028, PTC209, PTC596, or QW24.

### Pharmaceutical Kits

The present bifunctional compounds and/or compositions containing them may be assembled into kits or pharmaceutical systems. Kits or pharmaceutical systems according to this aspect of the disclosure may include a carrier or package such as a box, carton, tube or the like, having in close confinement therein one or more containers, such as vials, tubes, ampoules, or bottles, which contain bifunctional compound of the disclosure or a pharmaceutical composition thereof. The kits or pharmaceutical systems of the disclosure may also include printed instructions for using the compounds and compositions.

These and other aspects of the present disclosure will be further appreciated upon consideration of the following Examples, which are intended to illustrate certain particular embodiments of the disclosure but are not intended to limit its scope, as defined by the claims.

### EXAMPLES

These and other aspects of the present disclosure will be further appreciated upon consideration of the following Examples, which are intended to illustrate certain particular embodiments of the disclosure but are not intended to limit its scope, as defined by the claims.

### Synthesis of intermediates int-1 to int-3

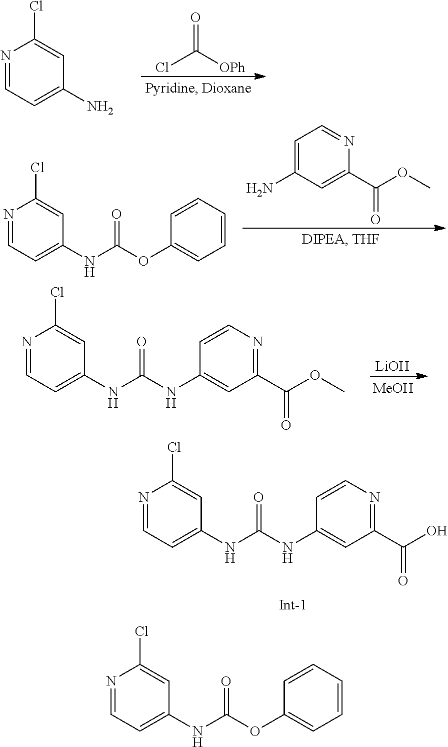

### Phenyl (3-chlorophenyl)carbamate

To a solution of 2-chloropyridin-4-amine (1 g, 7.78 mmol) and pyridine (0.63 mL, 7.78 mmol) in DCM (40 mL) was added phenyl chloroformate (1.03 mL, 8.17 mmol) dropwise. The reaction was stirred at room temperature for 16 hours (h). The reaction mixture was concentrated in vacuo and then diluted with ethyl acetate and water. The mixture was extracted three times with ethyl acetate. The combined organic layers were washed with brine, dried with sodium sulfate, filtered and concentrated in vacuo. The resulting residue was purified by silica-gel chromatography (EtOAc/hexane) to give phenyl (3-chlorophenyl)carbamate as a white solid (4.26 g, quantitative (quant.)).

MS (ESI) m/z 249.2 [M+H]⁺.

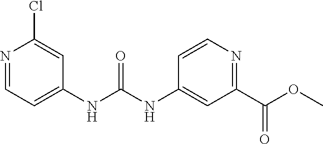 [SMILES: COC(=O)c1cc(NC(=O)Nc2ccnc(Cl)c2)ccn1]

### Methyl 4-(3-(2-chloropyridin-4-yl)ureido)picolinate

To a solution of phenyl (3-chlorophenyl)carbamate (350 mg, 1.41 mmol) and methyl 4-aminopicolinate (195 mg, 1.28 mmol) in THE was added N, N-diisopropylethylamine (DIPEA) (0.446 mL, 2.56 mmol), and the resulting mixture was microwaved at 110° C. for 45 minutes. The reaction mixture was concentrated in vacuo and purified by silica-gel chromatography (DCM/MeOH) to give methyl 4-(3-(2-chloropyridin-4-yl)ureido)picolinate as a yellow solid (157 mg, 33% yield).

MS (ESI) m/z 307.06 [M+H]⁺.

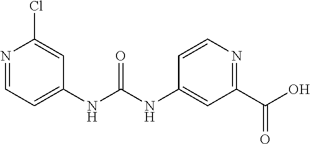 [SMILES: O=C(Nc1ccnc(Cl)c1)Nc1ccnc(C(=O)O)c1]

### 4-(3-(2-Chloropyridin-4-yl)ureido)picolinic acid (int-1)

To a solution of methyl 4-(3-(2-chloropyridin-4-yl)ureido)picolinate (130 mg, 0.43 mmol) in MeOH was added 2M LiOH (0.43 mL, 0.86 mmol), and the resulting reaction mixture was stirred at room temperature for 16 h. The mixture was concentrated in vacuo to give int-1 as a yellow solid (125 mg, 100% yield).

MS (ESI) m/z 293.04 [M+H]⁺.

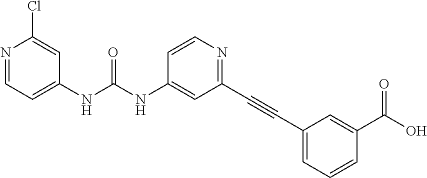 [SMILES: O=C(Nc1ccnc(Cl)c1)Nc1ccnc(C#Cc2cccc(C(=O)O)c2)c1]

### 3-((4-(3-(2-Chloropyridin-4-yl)ureido)pyridin-2-yl)ethynyl)benzoic acid (int-2)

Compound int-2 was synthesized according to the procedures in Papillon et al., J. Med. Chem. 61(22):10155-10172 (2018).

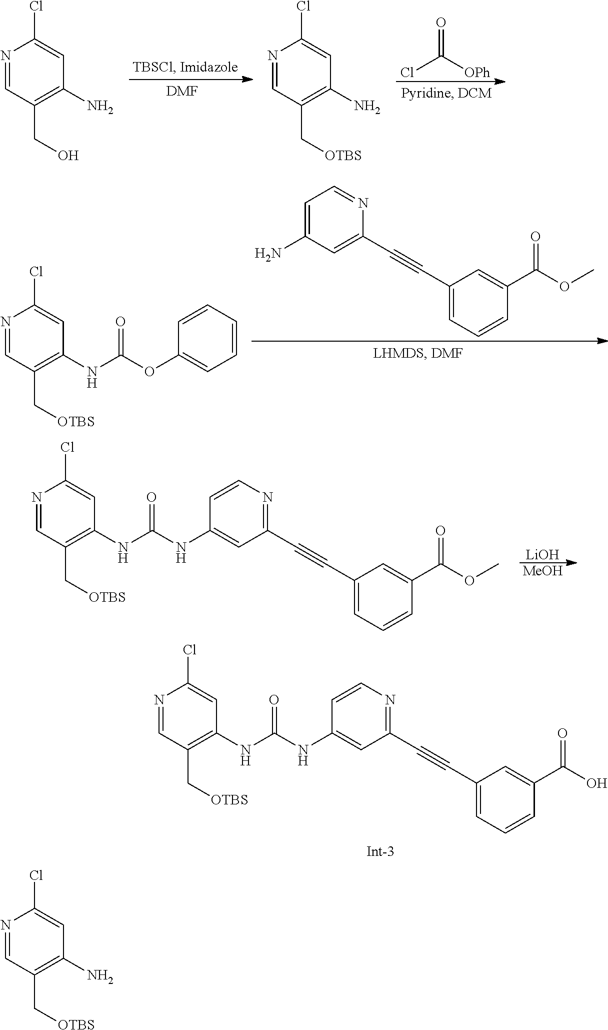

To a solution of (4-amino-6-chloropyridin-3-yl)methanol (1 g, 6.31 mmol) in DMF, tert-butyldimethylsilyl chloride (1.05 g, 6.94 mmol) and imidazole (1.07 g, 15.8 mmol) were added. The reaction mixture was stirred at room temperature for 3 h. The mixture was concentrated in vacuo and the resulting residue was extracted with ethyl acetate and water. The aqueous layer was with extracted 2 more times with ethyl acetate. The combined organic layers were washed with brine, dried with sodium sulfate, and concentrated in vacuo. The resulting crude product was purified by silica-gel chromatography (Hex/EtOAc) to give 5-(((tert-butyldimethylsilyl)oxy)methyl)-2-chloropyridin-4-amine as a white solid (1.63 g, 95% yield).

MS (ESI) m/z 273.3 [M+H]⁺.

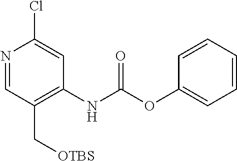 [SMILES: CC(C)(C)[Si](C)(C)OCc1cnc(Cl)cc1NC(=O)Oc1ccccc1]

### Phenyl (5-(((tert-butyldimethylsilyl)oxy)methyl)-2-chloropyridin-4-yl)carbamate

To a solution of 5-(((tert-butyldimethylsilyl)oxy)methyl)-2-chloropyridin-4-amine (1 g, 7.78 mmol) and pyridine (0.63 mL, 7.78 mmol) in DCM (40 mL) was added phenyl chloroformate (1.03 mL, 8.17 mmol) dropwise. The reaction was stirred at room temperature for 16 hours. Reaction was concentrated in vacuo then diluted with ethyl acetate and water. The mixture was extracted with ethyl acetate three times. The organic layers were combined, washed with brine, dried with sodium sulfate, filtered and concentrated. The residue was purified by silica-gel chromatography (EtOAc/hexane) to give 2, (5-(((tert-butyldimethylsilyl)oxy)methyl)-2-chloropyridin-4-yl)carbamate as white solid (4.26 g, quant.).

MS (ESI) m/z 393.2 [M+H]⁺.

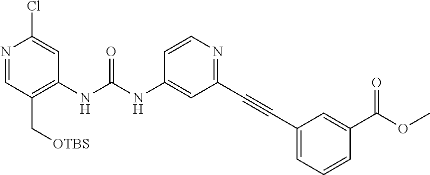 [SMILES: COC(=O)c1cccc(C#Cc2cc(NC(=O)Nc3cc(Cl)ncc3CO[Si](C)(C)C(C)(C)C)ccn2)c1]

### Methyl 3-((4-(3-(5-(((tert-butyldimethylsilyl)oxy)methyl)-2-chloropyridin-4-yl)ureido)pyridin-2-yl)ethynyl)benzoate

To a solution of (5-(((tert-butyldimethylsilyl)oxy)methyl)-2-chloropyridin-4-yl)carbamate (173 mg, 0.44 mmol) and DIPEA (0.14 mL, 0.8 mmol) in THF (3 mL) was added methyl 3-((4-aminopyridin-2-yl)ethynyl)benzoate (100 mg, 0.40 mmol). The reaction mixture was microwaved at 110° C. for 50 minutes and then concentrated in vacuo. The obtained residue was purified by silica-gel chromatography (EtOAc/hexane) to give methyl 3-((4-(3-(5-(((tert-butyldimethylsilyl)oxy)methyl)-2-chloropyridin-4-yl)ureido)pyridin-2-yl)ethynyl)benzoate as white solid (26 mg, 12% yield).

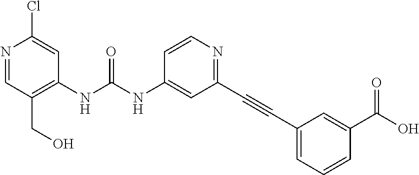 [SMILES: O=C(Nc1ccnc(C#Cc2cccc(C(=O)O)c2)c1)Nc1cc(Cl)ncc1CO]

### 3-((4-(3-(2-Chloro-5-(hydroxymethyl)pyridin-4-yl)ureido)pyridin-2-yl)ethynyl)benzoic acid (int-3)

To a solution of methyl 3-((4-(3-(5-(((tert-butyldimethylsilyl)oxy)methyl)-2-chloropyridin-4-yl)ureido)pyridin-2-yl)ethynyl)benzoate (26 mg, 0.05 mmol) in methanol (0.15 mL) was added 2M lithium hydroxide in water (0.05 mL, 0.1 mmol). The reaction was stirred at room temperature for 16 hours. The crude reaction mixture was concentrated in vacuo to give int-3 as white solid (21 mg, quant.).

### Example 2: Synthesis of 1-(2-chloropyridin-4-yl)-3-(2-(4-(2-((2-(2,6-dioxopiperidin-3-yl)-1,3-dioxoisoindolin-4-yl)amino)ethyl)piperazine-1-carbonyl)pyridin-4-yl)urea (YL-dS2-23, 1)

### General Procedure

To a solution of int-1, int-2, or int-3 (1 eq.) in dimethylformamide (DMF) (0.2M), triethylamine (TEA) (3 eq.) and HATU (2 eq.) were added, and the mixture stirred at room temperature for 15 minutes before the addition of the IMiD Amine (1 eq.). The resulting mixture reaction was stirred at room temperature for 16 h. The crude mixture was concentrated in vacuo and purified by silica-gel chromatography (DCM/MeOH) followed by high-performance liquid chromatography (HPLC) to give product (5-20% yield).

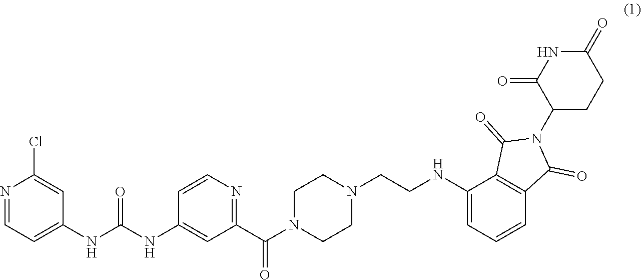 [SMILES: O=C1CCC(N2C(=O)c3cccc(NCCN4CCN(C(=O)c5cc(NC(=O)Nc6ccnc(Cl)c6)ccn5)CC4)c3C2=O)C(=O)N1]

Compound 1 was synthesized according to the above general procedure using int-1 (10 mg, 0.034 mmol) and IM-C₂-piperidine-1-Amine (13.2 mg, 0.034 mmol), and isolated as a yellow powder (2.4 mg, 11% yield).

MS (ESI) m/z 660.47 [M+H]⁺.

### Example 3: Synthesis of 4-(3-(2-chloropyridin-4-yl)ureido)-N-(8-((2-(2,6-dioxopiperidin-3-yl)-1,3-dioxoisoindolin-4-yl)amino)octyl)picolinamide (YL-dS2-24, 2)

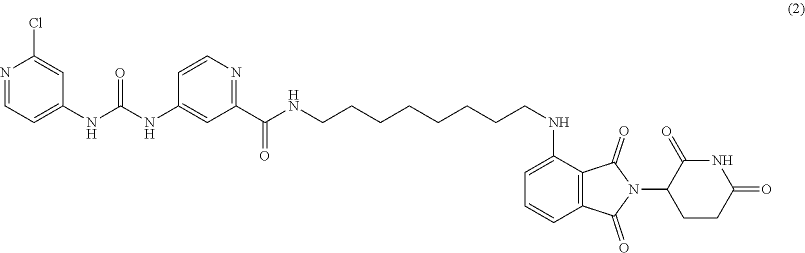 [SMILES: O=C1CCC(N2C(=O)c3cccc(NCCCCCCCCNC(=O)c4cc(NC(=O)Nc5ccnc(Cl)c5)ccn4)c3C2=O)C(=O)N1]

Compound 2 was synthesized in an analogous manner to compound 1 in Example 2 from int-1 (10 mg, 0.034 mmol) and IM-C₈-1-Amine (13.6 mg, 0.034 mmol), and isolated as a yellow powder (3.2 mg, 14% yield).

MS (ESI) m/z 675.44 [M+H]⁺.

### Example 4: Synthesis of 4-(3-(2-chloropyridin-4-yl)ureido)-N-(10-((2-(2,6-dioxopiperidin-3-yl)-1,3-dioxoisoindolin-4-yl)amino)decyl)picolinamide (YL-dS2-25, 3)

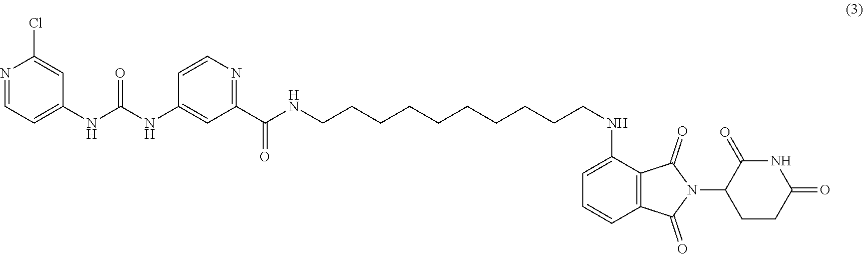 [SMILES: O=C1CCC(N2C(=O)c3cccc(NCCCCCCCCCCNC(=O)c4cc(NC(=O)Nc5ccnc(Cl)c5)ccn4)c3C2=O)C(=O)N1]

Compound 3 was synthesized in an analogous manner to compound 1 in Example 2 from int-1 (10 mg, 0.034 mmol) and IM-C₁₀-1-Amine (14.6 mg, 0.034 mmol), and isolated as a yellow powder (2.1 mg, 9% yield).

MS (ESI) m/z 703.44 [M+H]⁺.

### Example 5: Synthesis of 1-(2-chloropyridin-4-yl)-3-(2-((3-(4-(2-((2-(2,6-dioxopiperidin-3-yl)-1,3-dioxoisoindolin-4-yl)amino)ethyl)piperazine-1-carbonyl)phenyl)ethynyl)pyridin-4-yl)urea (YL-dS2-27, 4)

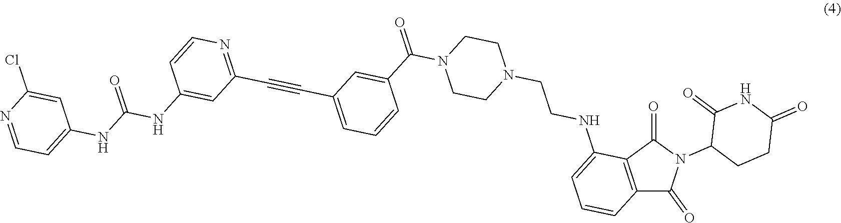 [SMILES: O=C1CCC(N2C(=O)c3cccc(NCCN4CCN(C(=O)c5cccc(C#Cc6cc(NC(=O)Nc7ccnc(Cl)c7)ccn6)c5)CC4)c3C2=O)C(=O)N1]

Compound 4 was synthesized in an analogous manner to compound 1 in Example 2 from int-2 (10 mg, 0.025 mmol) and IM-C₂-piperidine-1-Amine (12.4 mg, 0.025 mmol), and isolated as a yellow powder (1.8 mg, 10% yield).

MS (ESI) m/z 760.48 [M+H]⁺.

### Example 6: Synthesis of 3-(2-(4-(3-(2-chloropyridin-4-yl)ureido)pyridin-2-yl)ethyl)-N-(4-((2-(2,6-dioxopiperidin-3-yl)-1,3-dioxoisoindolin-4-yl)amino)butyl)benzamide (YL-dS2-28, 5)

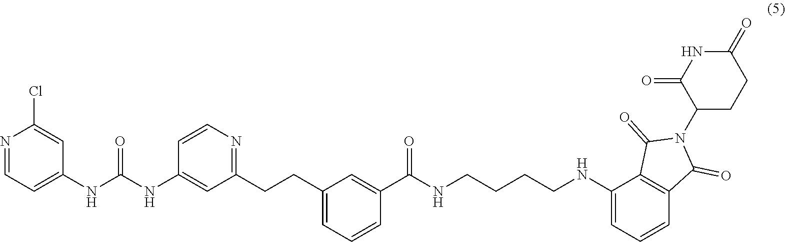 [SMILES: O=C1CCC(N2C(=O)c3cccc(NCCCCNC(=O)c4cccc(CCc5cc(NC(=O)Nc6ccnc(Cl)c6)ccn5)c4)c3C2=O)C(=O)N1]

To a stirred solution of compound 19 (example 15) (100 mg, 0.139 mmol) in DCM/MeOH (10 mL, 1:1) was added Pd/C (20 mg, 5 mol %), and the result mixture was stirred under H₂ atmosphere for 7 h. The reaction mixture was filtered through Celite®, and the filtrate was concentrated in vacuo to give a crude residue, which was purified by preparative (Prep)-HPLC to give compound 5 as a yellow solid (45.12 mg, 44.9% yield).

MS (ESI) m/z 722.24 [M+H]⁺.

### Example 7: Synthesis of 4-((4-(3-(2-chloropyridin-4-yl)ureido)pyridin-2-yl)ethynyl)-N-(4-((2-(2,6-dioxopiperidin-3-yl)-1,3-dioxoisoindolin-4-yl)amino)butyl)benzamide (YL-dS2-29, 6)

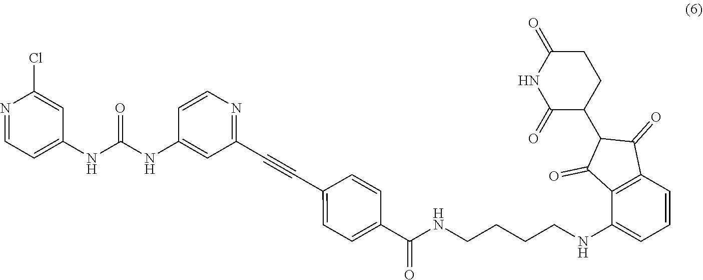 [SMILES: O=C1CCC(C2C(=O)c3cccc(NCCCCNC(=O)c4ccc(C#Cc5cc(NC(=O)Nc6ccnc(Cl)c6)ccn5)cc4)c3C2=O)C(=O)N1]

Compound 6 was synthesized in an analogous manner to compound 1 in Example 2 from a para-isomer of int-2 (16 mg, 0.041 mmol) and IM-C₄-1-Amine (14.1 mg, 0.041 mmol), and isolated as a yellow powder (6 mg, 21% yield).

MS (ESI) m/z 719.16 [M+H]⁺.

### Example 8: Synthesis of 4-(3-(2-chloropyridin-4-yl)ureido)-N-(12-((2-(2,6-dioxopiperidin-3-yl)-1,3-dioxoisoindolin-4-yl)amino)dodecyl)picolinamide (YL-dS2-30, 7)

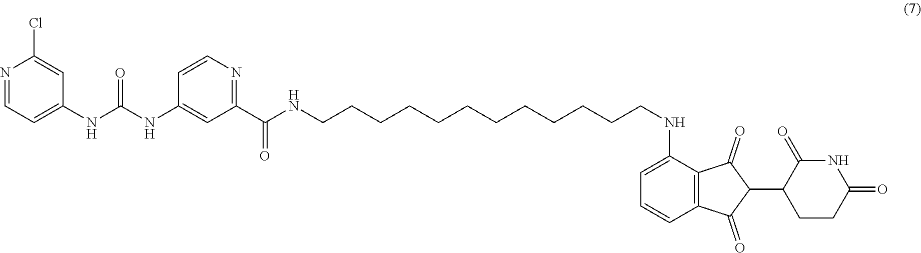 [SMILES: O=C1CCC(C2C(=O)c3cccc(NCCCCCCCCCCCCNC(=O)c4cc(NC(=O)Nc5ccnc(Cl)c5)ccn4)c3C2=O)C(=O)N1]

Compound 7 was synthesized in an analogous manner to compound 1 in Example 2 from int-1 (10 mg, 0.034 mmol) and IM-C₁₂-1-Amine (15.5 mg, 0.034 mmol), and isolated as a yellow powder (2 mg, 8% yield).

MS (ESI) m/z 730.3 [M+H]⁺.

### Example 9: Synthesis of 3-((4-(3-(2-chloro-5-(hydroxymethyl)pyridin-4-yl)ureido)pyridin-2-yl)ethynyl)-N-(4-((2-(2,6-dioxopiperidin-3-yl)-1,3-dioxoisoindolin-4-yl)amino)butyl)benzamide (YL-dS2-31, 8)

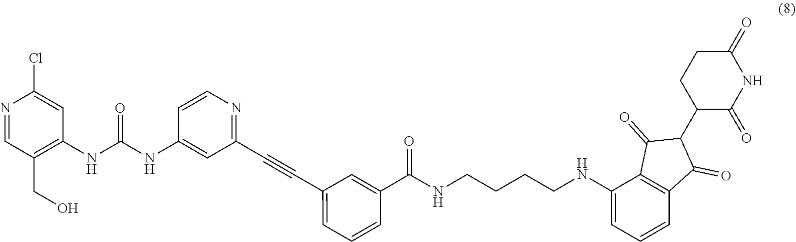 [SMILES: O=C1CCC(C2C(=O)c3cccc(NCCCCNC(=O)c4cccc(C#Cc5cc(NC(=O)Nc6cc(Cl)ncc6CO)ccn5)c4)c3C2=O)C(=O)N1]

Compound 8 was synthesized in an analogous manner to compound 1 in Example 2 from int-3 (10 mg, 0.019 mmol) and IM-C4-1-Amine (6.54 mg, 0.019 mmol), and isolated as a yellow powder (2 mg, 8% yield).

MS (ESI) m/z 730.3 [M+H]⁺.

### Example 10: Synthesis of 1-(2-chloropyridin-4-yl)-3-(2-((3-(4-(2-(2,6-dioxopiperidin-3-yl)-1,3-dioxoisoindolin-4-yl)piperazine-1-carbonyl)phenyl)ethynyl)pyridin-4-yl)urea (YL-dS2-32, 9)

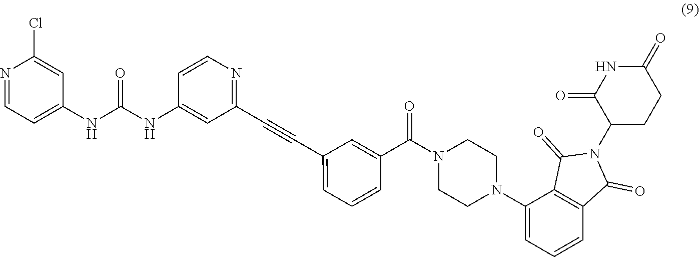 [SMILES: O=C1CCC(N2C(=O)c3cccc(N4CCN(C(=O)c5cccc(C#Cc6cc(NC(=O)Nc7ccnc(Cl)c7)ccn6)c5)CC4)c3C2=O)C(=O)N1]

Compound 9 was synthesized in an analogous manner to compound 1 in Example 2 from int-2 (100 mg, 0.25 mmol) and IM-piperidine-1-Amine (85.6 mg, 0.25 mmol), and isolated as a yellow powder (68.4 mg, 38% yield).

MS (ESI) m/z 717.19 [M+H]⁺.

### Example 11: Synthesis of 4-(3-(2-chloropyridin-4-yl)ureido)-N-(4-((2-(2,6-dioxopiperidin-3-yl)-1,3-dioxoisoindolin-4-yl)amino)butyl)picolinamide (YL-dS2-33, 10)

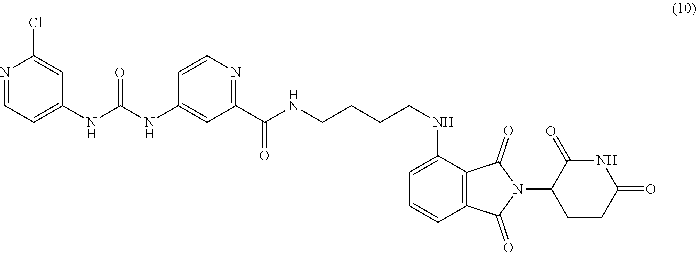 [SMILES: O=C1CCC(N2C(=O)c3cccc(NCCCCNC(=O)c4cc(NC(=O)Nc5ccnc(Cl)c5)ccn4)c3C2=O)C(=O)N1]

Compound 10 was synthesized in an analogous manner to compound 1 in Example 2 from int-1 (10 mg, 0.034 mmol) and IM-C4-1-Amine (11.8 mg, 0.034 mmol), and isolated as a yellow powder (1.6 mg, 8% yield).

MS (ESI) m/z 620.17 [M+H]⁺.

### Example 12: Synthesis of 1-(2-chloropyridin-4-yl)-3-(2-(3-(4-(2-(2,6-dioxopiperidin-3-yl)-1,3-dioxoisoindolin-4-yl)piperazine-1-carbonyl)phenethyl)pyridin-4-yl)urea YL-dS2-34, 11)

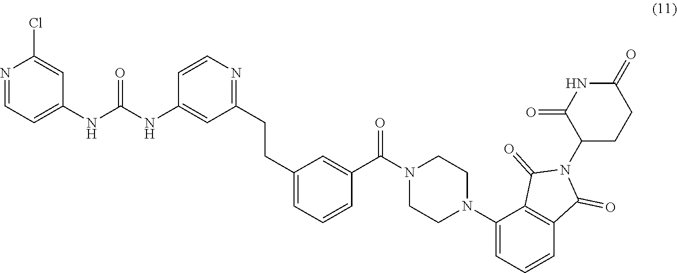 [SMILES: O=C1CCC(N2C(=O)c3cccc(N4CCN(C(=O)c5cccc(CCc6cc(NC(=O)Nc7ccnc(Cl)c7)ccn6)c5)CC4)c3C2=O)C(=O)N1]

To a stirred solution of compound 9 (10 mg, 0.014 mmol) in DCM/MeOH (10 mL, 1:1) was added Pd/C (1.5 mg, 10 mol %), and the resulting mixture was stirred under H₂ atmosphere for 8 h. The reaction mixture was filtered through Celite®, and the filtrate was concentrated in vacuo to give a crude residue, which was purified by HPLC to give compound 11 as a yellow solid (1.8 mg, 18% yield).

MS (ESI) m/z 722.24 [M+H]⁺.

### Example 13: Synthesis of 3-(2-(4-(3-(2-chloro-5-(hydroxymethyl)pyridin-4-yl)ureido)pyridin-2-yl)ethyl)-N-(4-((2-(2,6-dioxopiperidin-3-yl)-1,3-dioxoisoindolin-4-yl)amino)butyl)benzamide (YL-dS2-35, 12)

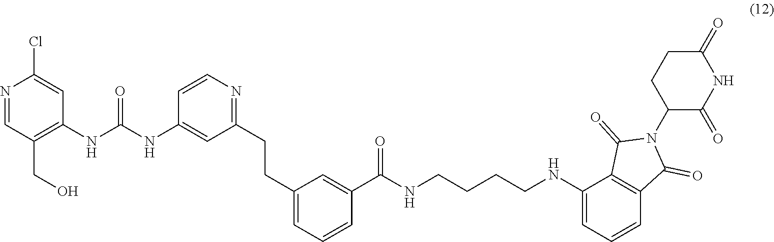 [SMILES: O=C1CCC(N2C(=O)c3cccc(NCCCCNC(=O)c4cccc(CCc5cc(NC(=O)Nc6cc(Cl)ncc6CO)ccn5)c4)c3C2=O)C(=O)N1]

To a stirred solution of compound 8 (10 mg, 0.013 mmol) in DCM/MeOH (10 mL, 1:1) was added Pd/C (1.5 mg, 10 mol %), and the resulting mixture was stirred under H₂ atmosphere for 8 h. The reaction mixture was filtered through Celite®, and the filtrate was concentrated in vacuo to give a crude residue, which was purified by HPLC to give compound 12 as a yellow solid (2 mg, 19% yield).

MS (ESI) m/z 753.25 [M+H]⁺.

### Example 14: Synthesis of 4-(3-(2-chloropyridin-4-yl)ureido)-N-(4-(((2-(2,6-dioxopiperidin-3-yl)-1,3-dioxoisoindolin-4-yl)amino)methyl)benzyl)picolinamide (YL-dS2-36, 13)

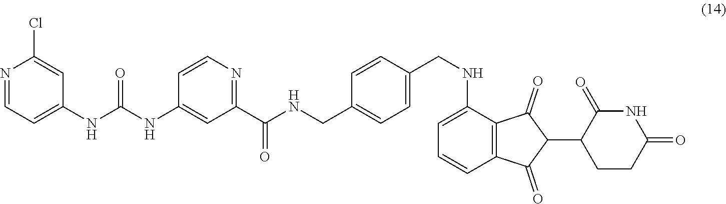 [SMILES: O=C1CCC(C2C(=O)c3cccc(NCc4ccc(CNC(=O)c5cc(NC(=O)Nc6ccnc(Cl)c6)ccn5)cc4)c3C2=O)C(=O)N1]

Compound 14 was synthesized in an analogous manner to compound 1 in Example 2 from int-1 (10 mg, 0.034 mmol) and IM-Benzyl-C₁-Amine (13.4 mg, 0.034 mmol), and isolated as a yellow powder (1.3 mg, 6% yield).

MS (ESI) m/z 667.18 [M+H]⁺.

### Example 15: Synthesis of 3-((4-(3-(2-chloropyridin-4-yl)ureido)pyridin-2-yl)ethynyl)-N-(4-((2-(2,6-dioxopiperidin-3-yl)-1,3-dioxoisoindolin-4-yl)amino)butyl)benzamide (DL-dS2-4, 19)

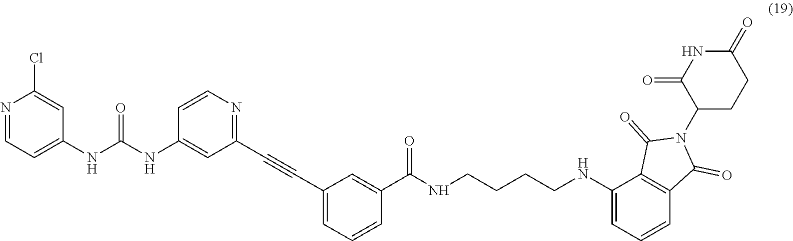 [SMILES: O=C1CCC(N2C(=O)c3cccc(NCCCCNC(=O)c4cccc(C#Cc5cc(NC(=O)Nc6ccnc(Cl)c6)ccn5)c4)c3C2=O)C(=O)N1]

Compound 19 was synthesized in an analogous manner to compound 1 in Example 2 from int-2 (10 mg, 0.034 mmol) and IM-C4-1-Amine (11.8 mg, 0.034 mmol), and isolated as a yellow powder.

### Example 16: Cellular degradation of SMARCA4 in NOMO-1 cells with bifunctional compounds 1-6

For each tested condition, 1 million cells were seeded into a well of a 6-well plate. After overnight incubation at 37° C. in 5% CO₂, cells were treated with either DMSO or varying doses of the indicated bifunctional compound and put back into the incubator for the indicated treatment time. Treated cells were collected by transferring cell suspensions from 6-well plate wells into microcentrifuge tubes and spinning down samples at 3000 rpm for 3 minutes. Supernatant was discarded and all samples were washed with phosphate-buffered saline (PBS) to remove any remaining compound from the cells. Cells were lysed using radioimmunoprecipitation assay (RIPA) buffer and sonicated on ice for 3 seconds at 25% amplification and allowed to rest for 15-30 minutes. Lysates were then spun down at 15000 rpm for 10 minutes and supernatant was collected. Protein concentration was measured using the Pierce™ bicinchoninic acid (BCA) Protein Assay Kit from ThermoFisher Scientific according to the manufacturer's instructions. Equivalent amounts of protein were resolved by western blotting using 4-12% Bis-Tris NuPAGE™ gels (Thermo-Fisher Scientific) prior to transfer and overnight immunoblotting using primary antibodies to the proteins indicated. Secondary antibodies were horseradish peroxidase (HRP)-conjugated anti-rabbit or anti-mouse (Santa Cruz Biotechnology®) and incubated at room temperature for 1 hour prior to exposure to enhanced chemiluminescence reagents (GE Amersham).

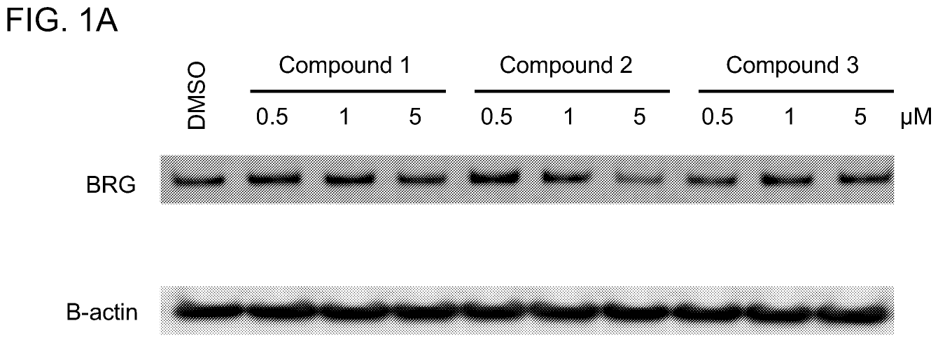

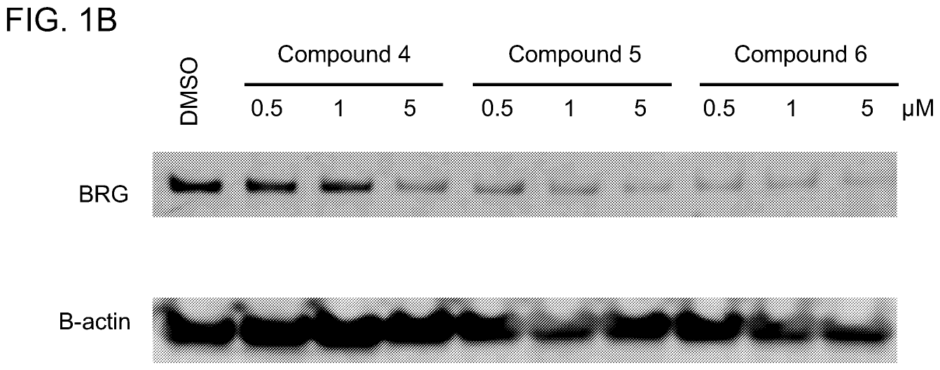

The results of are illustrated in FIG. 1A-1B. They show that BRG (SMARCA4) was significantly degraded after low micromolar treatment of bifunctional compounds 5 and 6 at 24 hours in NOMO1 cells.

### Example 17: Cell proliferation assays in MV411 (human myelomonocytic leukemia) and MOLM13 (acute monocytic leukemia) cells with bifunctional compounds 16-15

2000 cells were seeded in 50 μL of RPMI medium in each well of a white, solid bottomed 384-well plate. Plates were spun down briefly, and seeded cells were pinned with 100 nM of the bifunctional compound at serial three-fold diluted concentrations using the JANUS® Liquid Handling Workstation. Plates were incubated at 37° C. in 5% CO₂ for the indicated treatment time (48 hours). After compound treatment, 15 μL of 1:2 solution of ATPlite™ 1Step diluted in PBS was added to each well of the 384-well plates. Plates were put on a plate shaker for 1 minute and incubated at room temperature for 9 minutes, and the resulting luminescence signal was read using an EnVision® plate reader. Cell viability was calculated by normalizing luminescence signals of SMARC compound treated cells to that of vehicle treated cells.

The results are illustrated below in Table 1.

TABLE 1
IC₅₀ values in MV411 and MOLM13 cells.

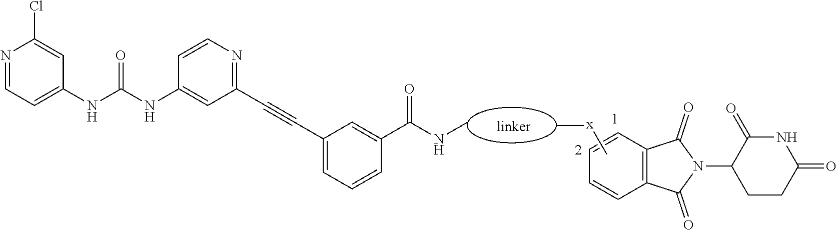

| | | | | IC₅₀ (μM) | |
|---|---|---|---|---|---|
| Compound | linker | X | Position | MV411-48h | MOLM13-48h |
| 16 | 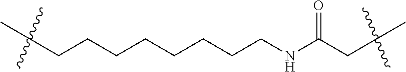 [SMILES: *CCCCCCCCNC(=O)C*] | O | 1 | 1778 | — |
| 17 | 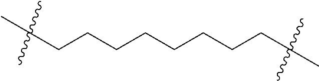 [SMILES: *CCCCCCCC*] | NH | 1 | 0.4908 | 0.5661* |
| 18 | 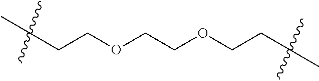 [SMILES: *CCOCCOCC*] | NH | 1 | 0.1092 | 1.283* |
| 19 | 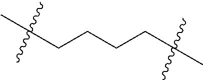 [SMILES: *CCCC*] | NH | 1 | 0.1092 | 0.0571 |
| 20 | 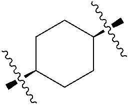 | NH | 1 | n/a | 94.19 |
| 21 | 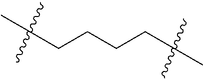 [SMILES: *CCCC*] | NH | 2 | 0.01024 | 0.07926 |
| 22 | 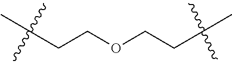 [SMILES: *CCOCC*] | NH | 2 | 0.03705 | 0.253 |
| 23 | 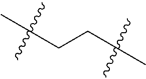 [SMILES: *CC*] | NH | 1 | 21.71 | 6.177 |
| 24 | 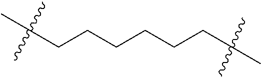 [SMILES: *CCCCCC*] | NH | 1 | 0.03895 | 0.3437 |
| 25 | 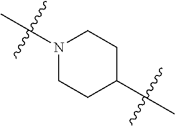 [SMILES: *C1CCN(*)CC1] | NH | 1 | 248.8 | n/a |

*72 h
n/a not acquired

The results in Table 1 show IC₅₀ values of cell viability in MV411 and MOLM13 cells after being treated with bifunctional compounds of various linkers and linker positions at 48 hours, in which bifunctional compound 19 exhibited submicromolar for both cell lines.

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present invention as defined by the appended claims.

What is claimed is:

1. A bifunctional compound having a structure represented by formula (I): 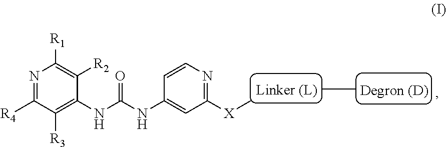
or a pharmaceutically acceptable salt or stereoisomer thereof, wherein 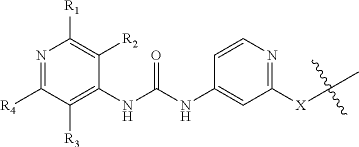
represents a targeting moiety that binds SWI/SNF related, matrix associated, actin dependent regulator of chromatin, subfamily a, member 4 (SMARCA4) or SMARCA2 and SMARCA4, the degron represents a moiety that binds an E3 ubiquitin ligase and L represents a linker that covalently attaches the targeting moiety to the degron, wherein:
R₁ and R₄ are each independently H or halogen;
R₂ is H or NH₂;
R₃ is H, NH₂, NO₂, OH, CN, (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different Ria groups, wherein each Ria is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
X is C(O), C(O)NH 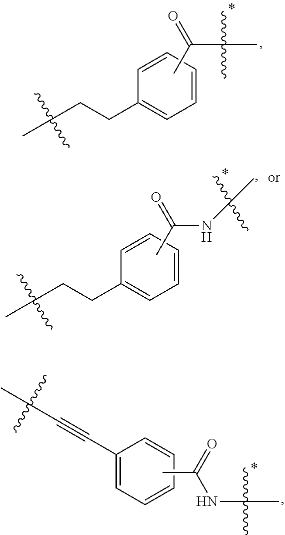
wherein the squiggle ( [symbol] ) is the point of attachment to the pyridine ring and the squiggle-asterisk ( [symbol] ) is the point of attachment to the linker.

2. The bifunctional compound of claim 1, R₃ is H, NH₂, or CH₂OH.

3. The bifunctional compound of claim 1, wherein R₁ is halogen, and R₂, R₃, R₄ are H.

4. The bifunctional compound of claim 1, wherein R₁ is halogen, R₂ and R₄ are H, and R₃ is CH₂OH.

5. The bifunctional compound of claim 3, wherein R₁ is Cl or F.

6. The bifunctional compound of claim 1, wherein R₁ and R₃ are H, R₂ is NH₂, and R₄ is halogen.

7. The bifunctional compound of claim 6, wherein R₄ is C₁.

8. The bifunctional compound of claim 1, wherein X is C(O), C(O)NH,

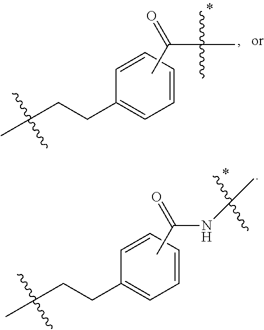

9. The bifunctional compound of claim 7, wherein X is

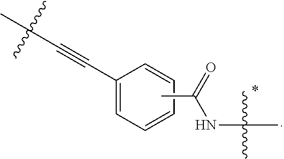

10. The bifunctional compound of claim 1, wherein R₁ is halogen and R₃ is H, NH₂, or methoxy, and X is not

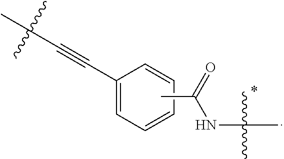

11. The bifunctional compound of claim 1, which has any one of structures I-1 to I-17: 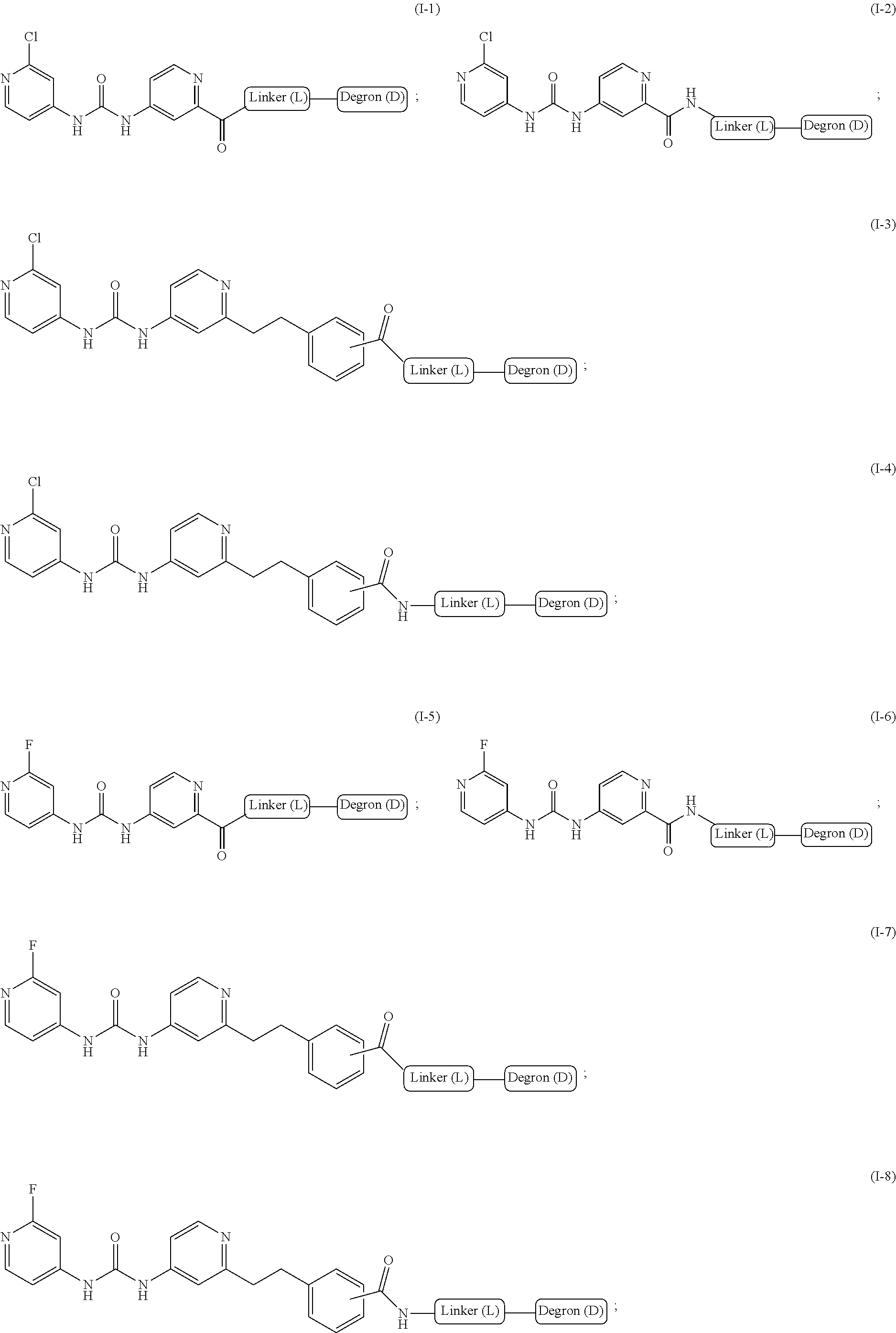 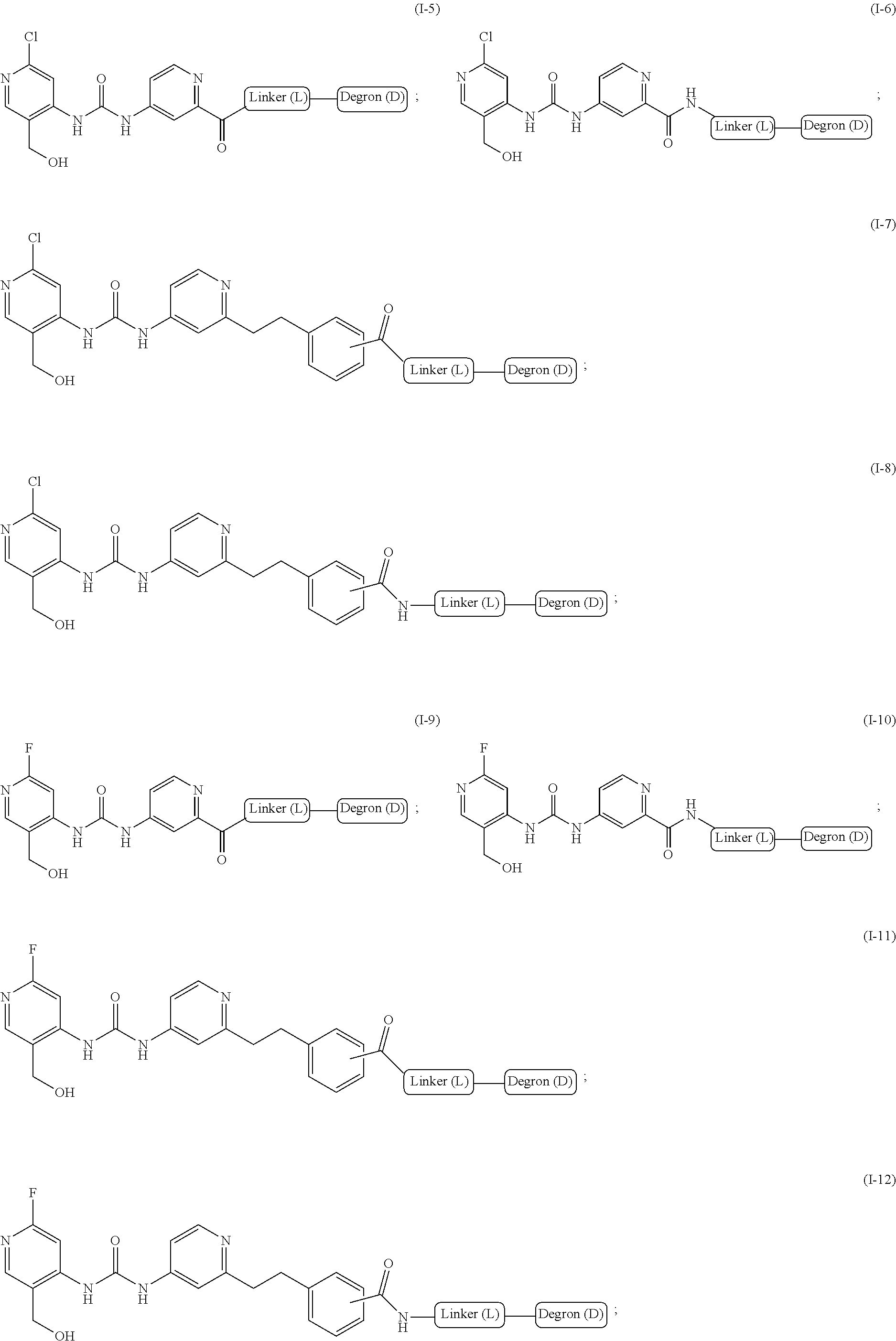 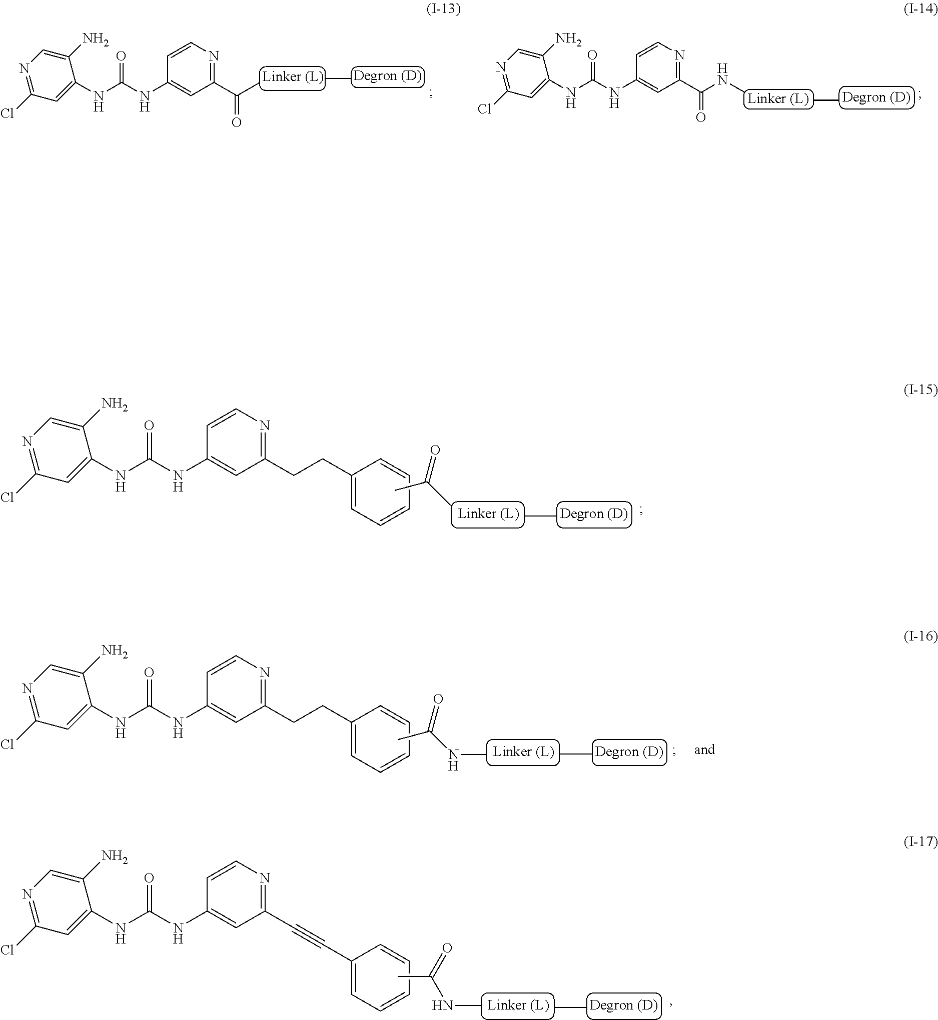
or a pharmaceutically acceptable salt or stereoisomer thereof

12. The bifunctional compound of claim 1, wherein the linker comprises an uninterrupted C₂ to C₁₅ alkylene chain.

13. The bifunctional compound of claim 12, wherein the alkylene chain comprises 2-10 uninterrupted alkylene units.

14. The bifunctional compound of claim 1, wherein the linker comprises 1-6 uninterrupted PEG units.

15. The bifunctional compound of claim 1, wherein the linker further comprises at least one group selected from —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, and 5- to 12-membered heteroarylene, wherein R′ is H or C₁-C₆ alkylene, and wherein the groups may be the same or different.

16. The bifunctional compound of claim 1, wherein the linker comprises a C₆ carbocyclene, 6-membered heterocyclene, or phenyl group.

17. The bifunctional compound of claim 16, wherein the 6-membered heterocyclene is

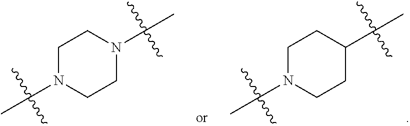 [SMILES: *N1CCN(*)CC1 ; *C1CCN(*)CC1]

18. The bifunctional compound of claim 1, wherein the linker is any one of structures:

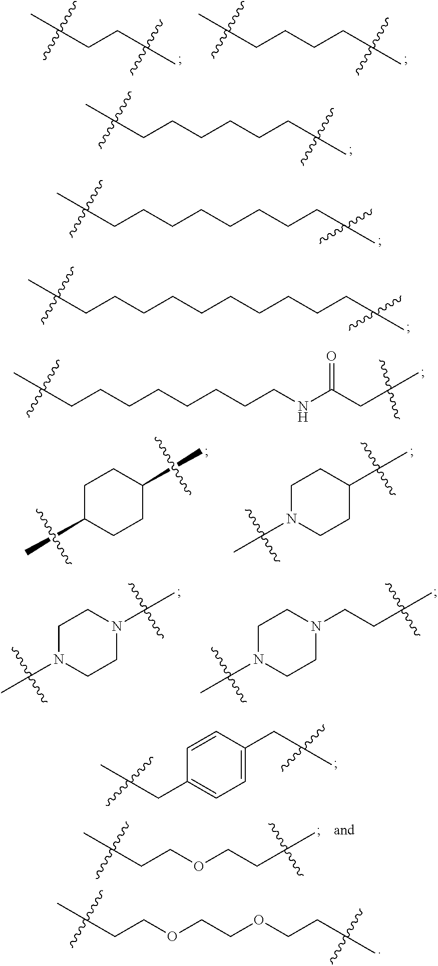

19. The bifunctional compound of claim 1, wherein the degron is represented by any one of structures (D1a) to (D1d): 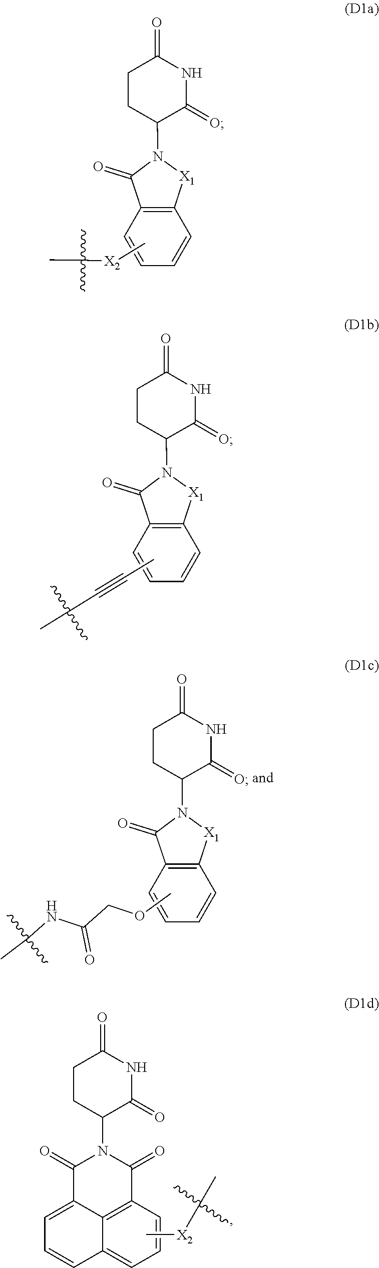
wherein X₁ is CH₂ or C(O) and X₂ is a bond, NH, or O.

20. (canceled)

21. The bifunctional compound of claim 1, wherein the degron is represented by any one of structures (D2-a) to (D2-f): 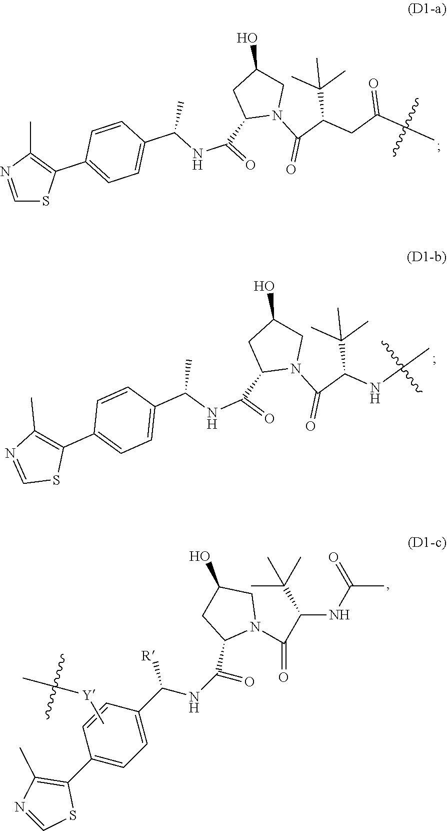
wherein Y′ is a bond, N, O or C and R′ is H or methyl; 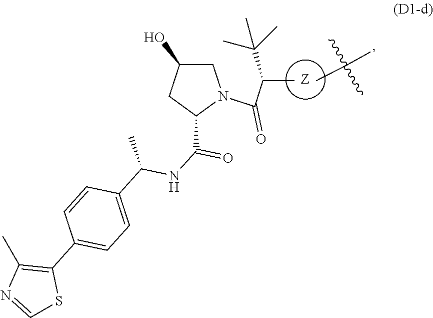
wherein Z is a C₅-C₆ carbocyclic or a C₅-C₆ heterocyclic group; 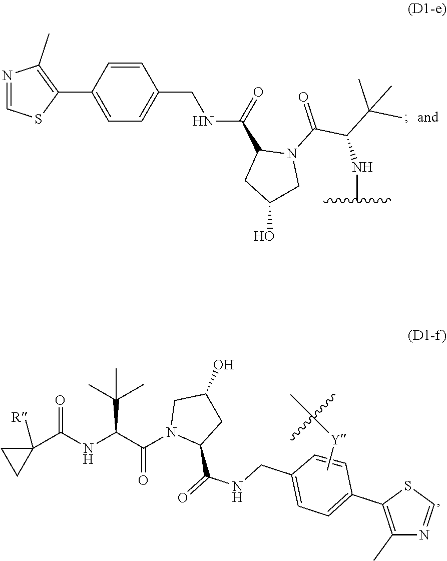
wherein Y″ is a bond, N, O or C and R″ is F or CN, or a stereoisomer thereof.

22. The bifunctional compound of claim 21, wherein Z is

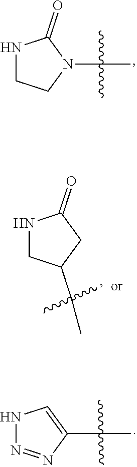

23. A bifunctional compound, which is any one of structures (1) to (27): 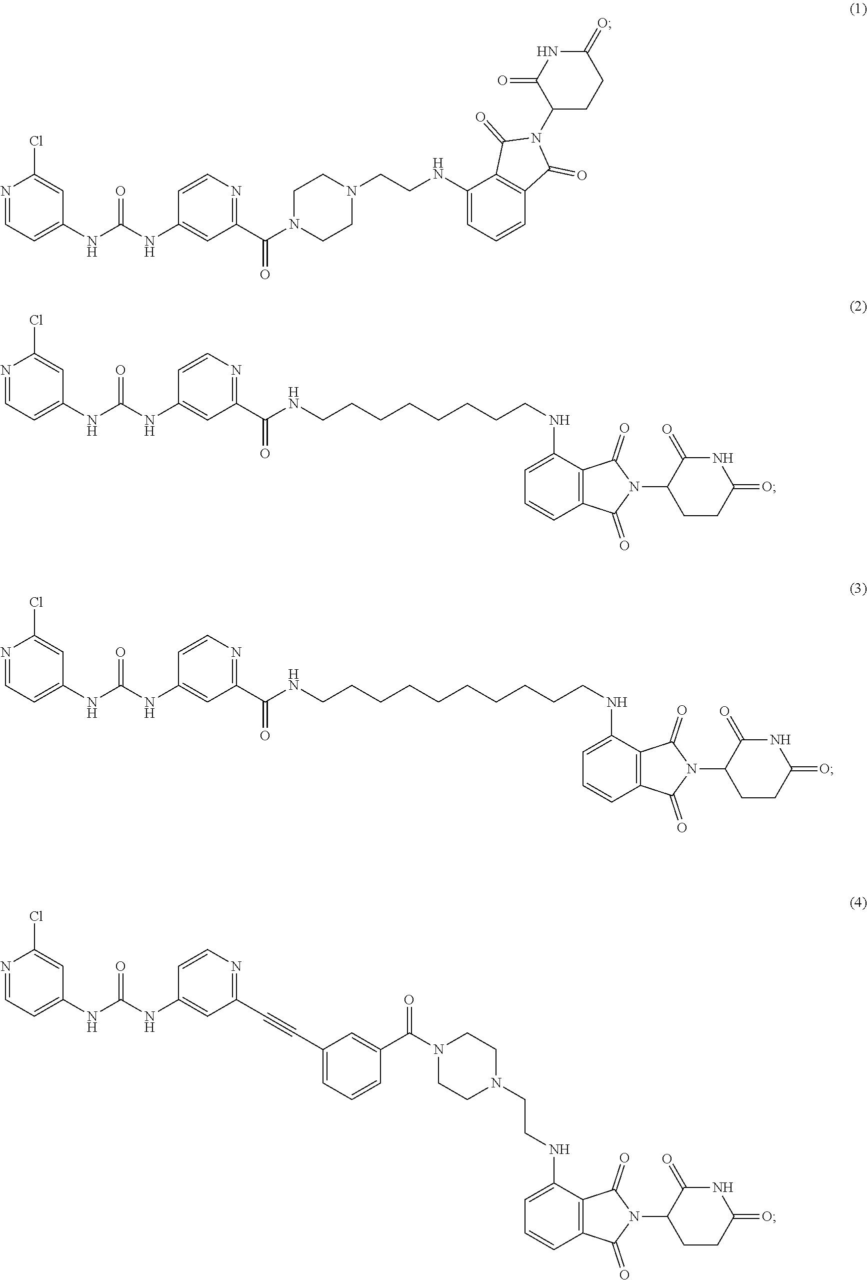 [SMILES: O=C1CCC(N2C(=O)c3cccc(NCCN4CCN(C(=O)c5cc(NC(=O)Nc6ccnc(Cl)c6)ccn5)CC4)c3C2=O)C(=O)N1 ; O=C1CCC(N2C(=O)c3cccc(NCCCCCCCCNC(=O)c4cc(NC(=O)Nc5ccnc(Cl)c5)ccn4)c3C2=O)C(=O)N1 ; O=C1CCC(N2C(=O)c3cccc(NCCCCCCCCCCNC(=O)c4cc(NC(=O)Nc5ccnc(Cl)c5)ccn4)c3C2=O)C(=O)N1 ; O=C1CCC(N2C(=O)c3cccc(NCCN4CCN(C(=O)c5cccc(C#Cc6cc(NC(=O)Nc7ccnc(Cl)c7)ccn6)c5)CC4)c3C2=O)C(=O)N1] 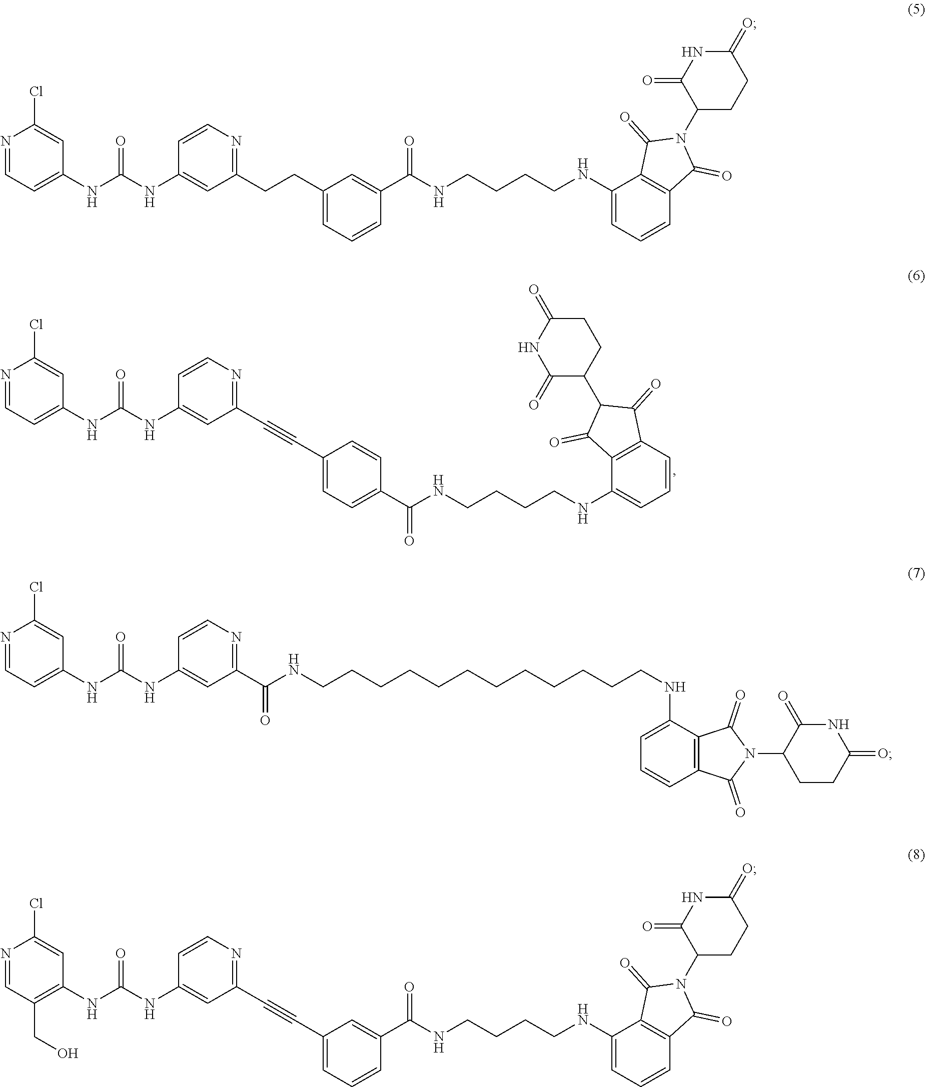 [SMILES: O=C1CCC(N2C(=O)c3cccc(NCCCCNC(=O)c4cccc(CCc5cc(NC(=O)Nc6ccnc(Cl)c6)ccn5)c4)c3C2=O)C(=O)N1 ; O=C1CCC(C2C(=O)c3cccc(NCCCCNC(=O)c4ccc(C#Cc5cc(NC(=O)Nc6ccnc(Cl)c6)ccn5)cc4)c3C2=O)C(=O)N1 ; O=C1CCC(N2C(=O)c3cccc(NCCCCCCCCCCCCNC(=O)c4cc(NC(=O)Nc5ccnc(Cl)c5)ccn4)c3C2=O)C(=O)N1 ; O=C1CCC(N2C(=O)c3cccc(NCCCCNC(=O)c4cccc(C#Cc5cc(NC(=O)Nc6cc(Cl)ncc6CO)ccn5)c4)c3C2=O)C(=O)N1] 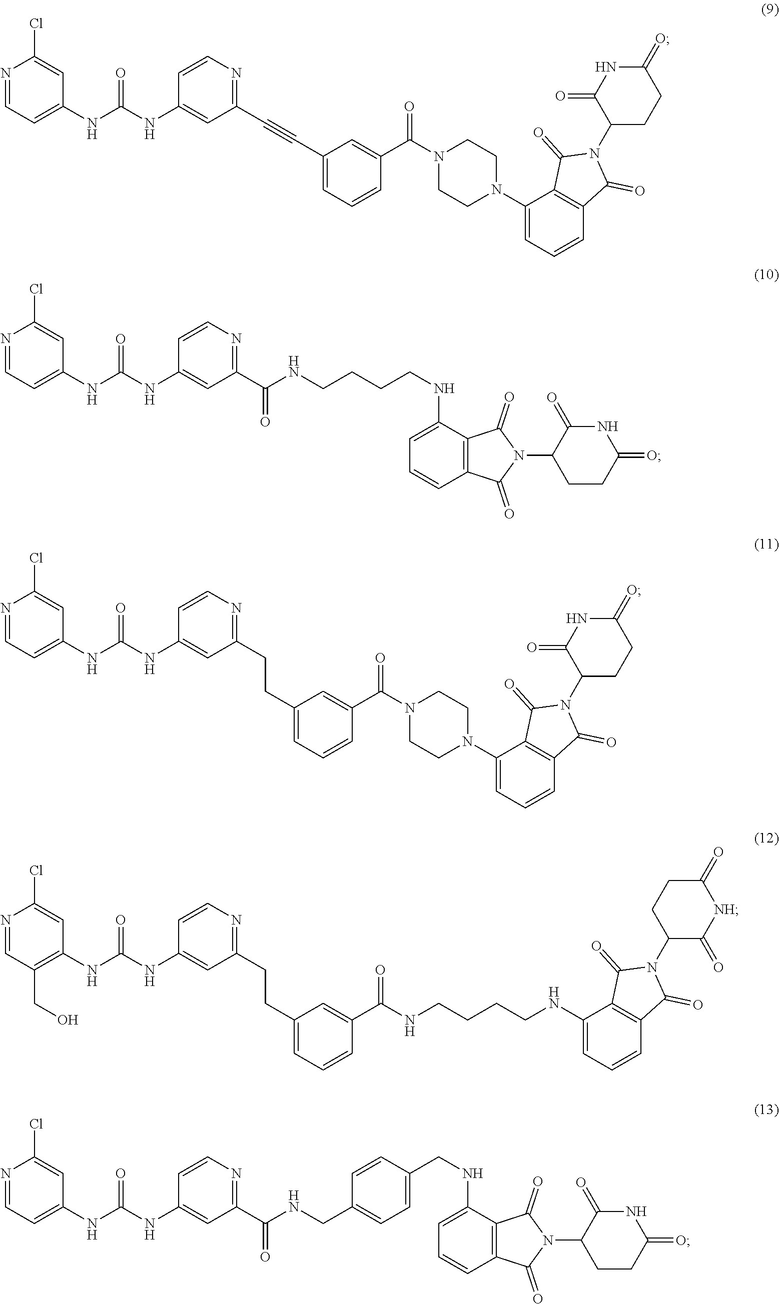 [SMILES: O=C1CCC(N2C(=O)c3cccc(N4CCN(C(=O)c5cccc(C#Cc6cc(NC(=O)Nc7ccnc(Cl)c7)ccn6)c5)CC4)c3C2=O)C(=O)N1 ; O=C1CCC(N2C(=O)c3cccc(NCCCCNC(=O)c4cc(NC(=O)Nc5ccnc(Cl)c5)ccn4)c3C2=O)C(=O)N1 ; O=C1CCC(N2C(=O)c3cccc(N4CCN(C(=O)c5cccc(CCc6cc(NC(=O)Nc7ccnc(Cl)c7)ccn6)c5)CC4)c3C2=O)C(=O)N1 ; O=C1CCC(N2C(=O)c3cccc(NCCCCNC(=O)c4cccc(CCc5cc(NC(=O)Nc6cc(Cl)ncc6CO)ccn5)c4)c3C2=O)C(=O)N1 ; O=C1CCC(N2C(=O)c3cccc(NCc4ccc(CNC(=O)c5cc(NC(=O)Nc6ccnc(Cl)c6)ccn5)cc4)c3C2=O)C(=O)N1] 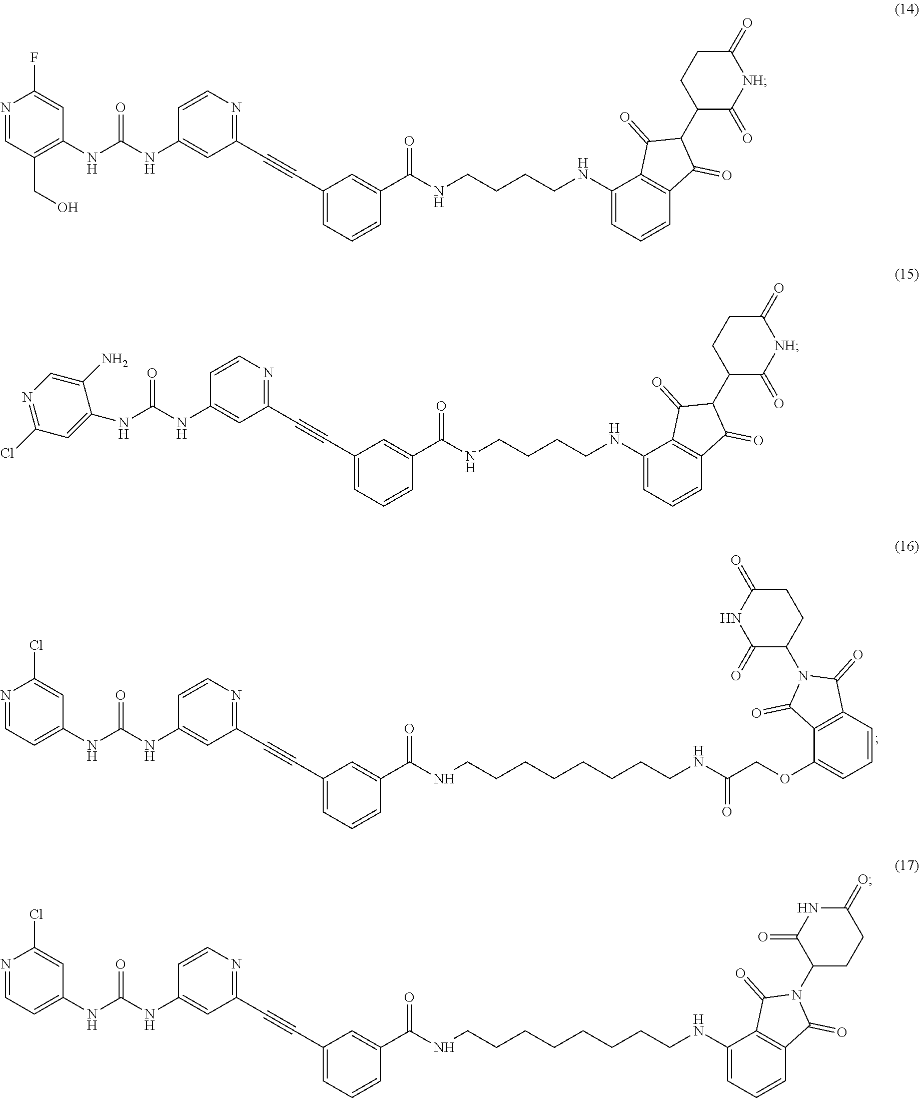 [SMILES: O=C1CCC(C2C(=O)c3cccc(NCCCCNC(=O)c4cccc(C#Cc5cc(NC(=O)Nc6cc(F)ncc6CO)ccn5)c4)c3C2=O)C(=O)N1 ; Nc1cnc(Cl)cc1NC(=O)Nc1ccnc(C#Cc2cccc(C(=O)NCCCCNc3cccc4c3C(=O)C(C3CCC(=O)NC3=O)C4=O)c2)c1 ; O=C(COc1cccc2c1C(=O)N(C1CCC(=O)NC1=O)C2=O)NCCCCCCCCNC(=O)c1cccc(C#Cc2cc(NC(=O)Nc3ccnc(Cl)c3)ccn2)c1 ; O=C1CCC(N2C(=O)c3cccc(NCCCCCCCCNC(=O)c4cccc(C#Cc5cc(NC(=O)Nc6ccnc(Cl)c6)ccn5)c4)c3C2=O)C(=O)N1] 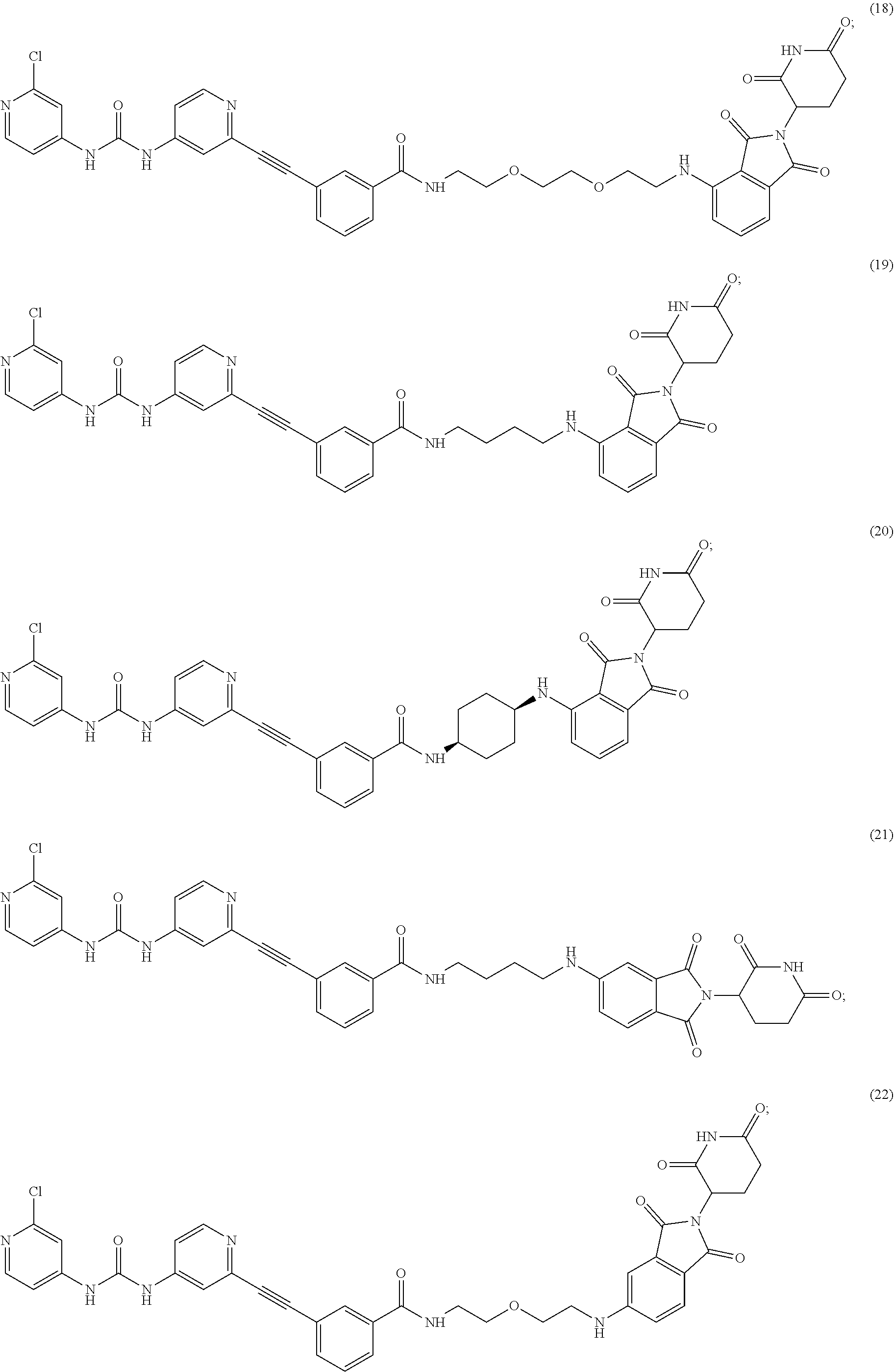 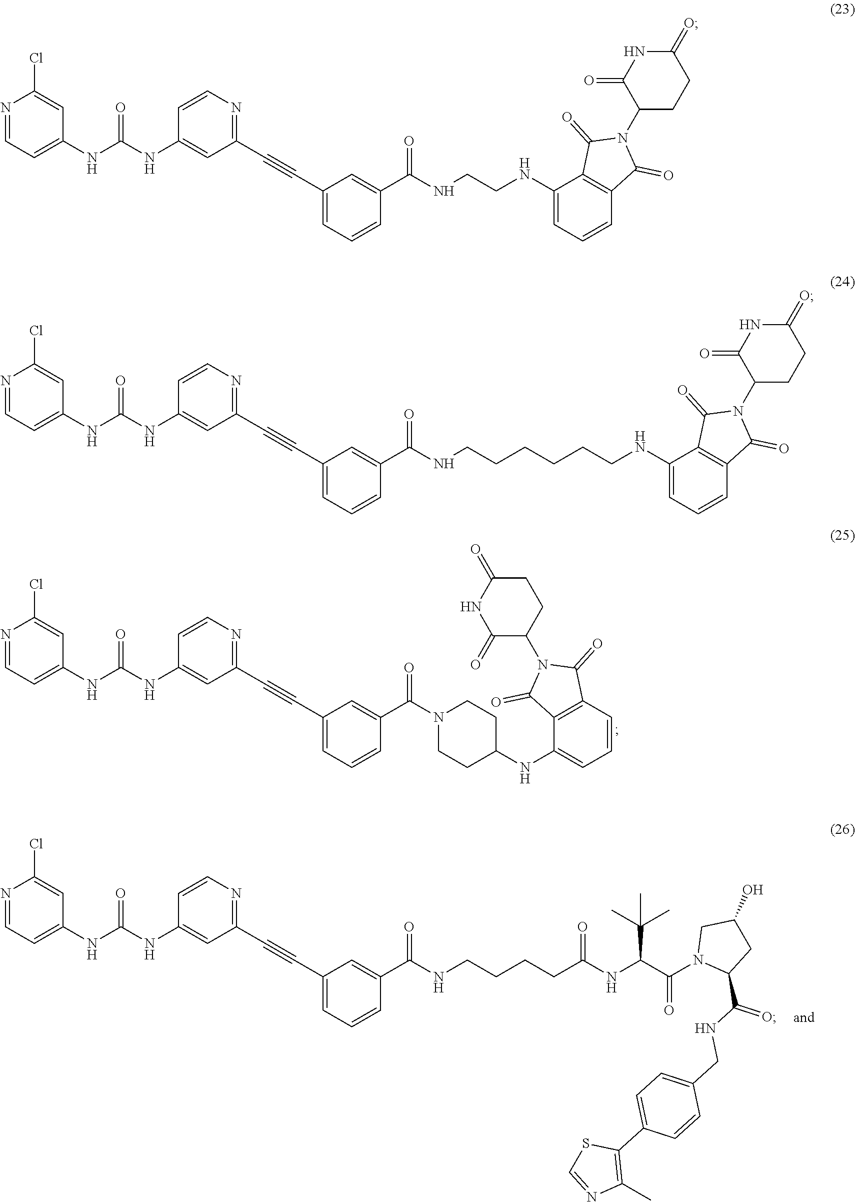 [SMILES: O=C1CCC(N2C(=O)c3cccc(NCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NCCCCCCNC(=O)c4cccc(C#Cc5cc(NC(=O)Nc6ccnc(Cl)c6)ccn5)c4)c3C2=O)C(=O)N1 ; O=C1CCC(N2C(=O)c3cccc(NC4CCN(C(=O)c5cccc(C#Cc6cc(NC(=O)Nc7ccnc(Cl)c7)ccn6)c5)CC4)c3C2=O)C(=O)N1 ; Cc1ncsc1-c1ccc(CNC(=O)[C@@H]2C[C@@H](O)CN2C(=O)[C@@H](NC(=O)CCCCNC(=O)c2cccc(C#Cc3cc(NC(=O)Nc4ccnc(Cl)c4)ccn3)c2)C(C)(C)C)cc1] 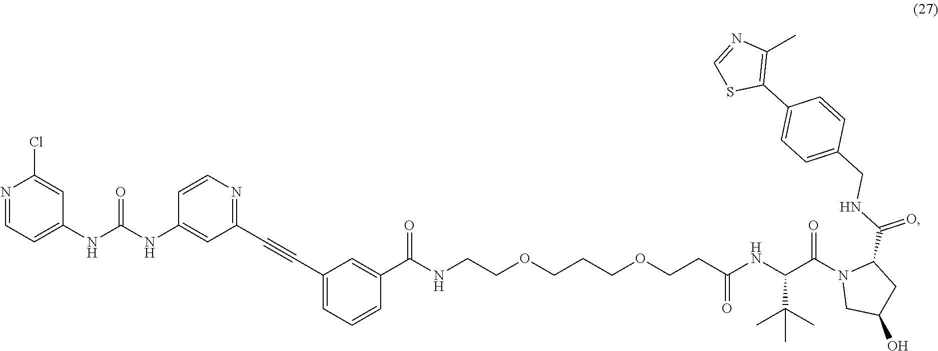 [SMILES: Cc1ncsc1-c1ccc(CNC(=O)[C@@H]2C[C@@H](O)CN2C(=O)[C@@H](NC(=O)CCOCCCOCCNC(=O)c2cccc(C#Cc3cc(NC(=O)Nc4ccnc(Cl)c4)ccn3)c2)C(C)(C)C)cc1]
or a pharmaceutically acceptable salt or stereoisomer thereof.

24. A pharmaceutical composition, comprising a therapeutically effective amount of the bifunctional compound or pharmaceutically acceptable salt or stereoisomer thereof of claim 1, and a pharmaceutically acceptable carrier.

25. A method of treating a disease or disorder that is characterized or mediated by aberrant activity of SMARCA4 or SMARCA2 and SMARCA4, comprising administering to a subject in need thereof a therapeutically effective amount of the bifunctional compound or pharmaceutically acceptable salt or stereoisomer thereof of claim 1.

26. The method of claim 25, wherein the disease or disorder is cancer.

27. The method of claim 26, wherein the cancer is selected from synovial sarcoma, lung cancer, ovarian cancer, brain cancer, kidney cancer, leukemia, non-small cell lung cancer, Burkitt's Lymphoma, childhood medulloblastoma, pancreatic adenocarcinoma, ovarian clear cell carcinoma, renal cell carcinoma, endometrial carcinoma, and melanoma.

28. A method of treating a diffuse midline glioma harboring a H3K27M mutation, comprising administering to a subject in need thereof a therapeutically effective amount of the bifunctional compound or pharmaceutically acceptable salt or stereoisomer thereof of claim 1.

29. A method of treating a diffuse midline glioma harboring a H3K27M mutation, comprising administering to a subject in need thereof a therapeutically effective amount of a bifunctional compound of formula I′, I″ or II″, or a pharmaceutically acceptable salt or stereoisomer thereof: 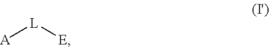
wherein
A is 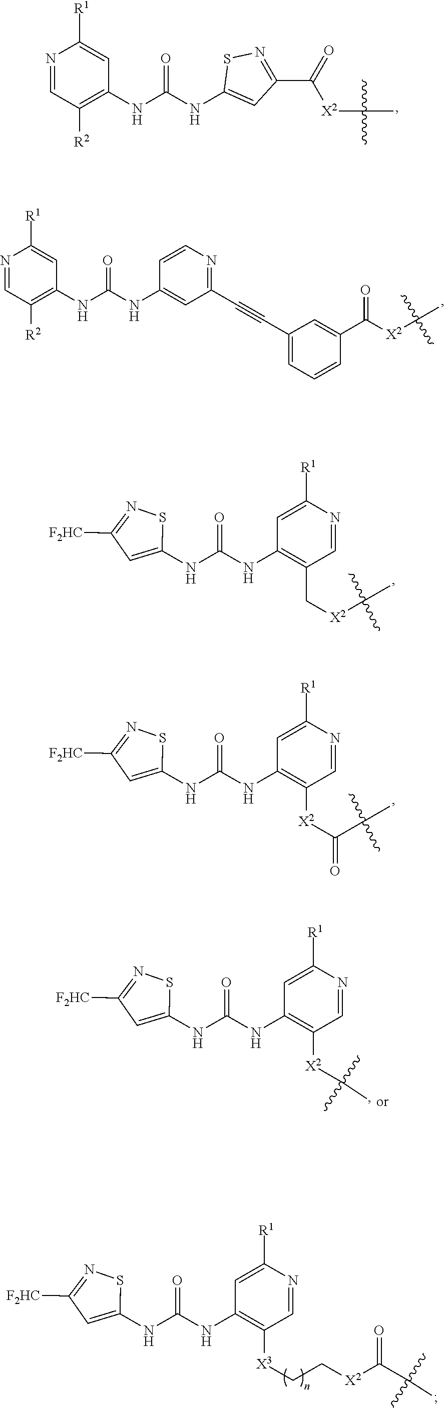 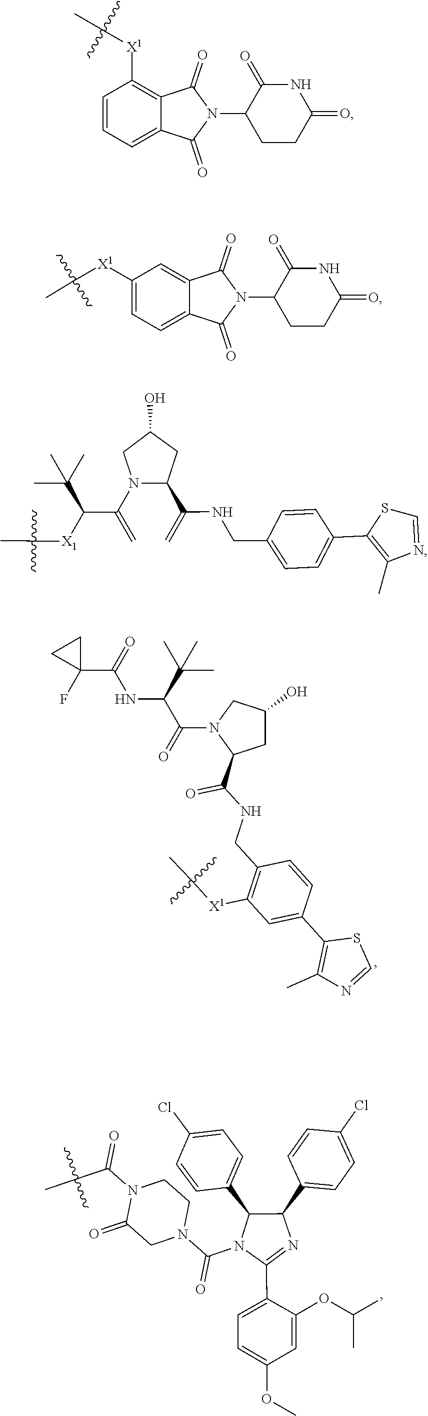 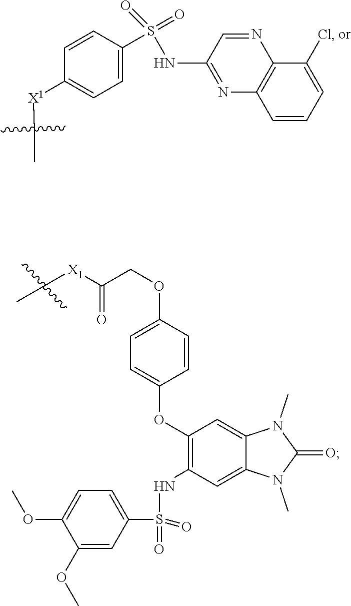
X¹ and X² are each independently selected from O, NR³, and S; or X¹ or X² combine with L to form a heterocyclyl or heteroaryl; or both of X¹ and X² combine with L to form a heterocyclyl or heteroaryl;
X³ is O, NR³, or S;
L comprises an alkylene, alkenylene, alkynylene, cycloalkylene, heterocyclylene, arylene, or heteroarylene chain, or a combination thereof, comprising 1 to 35 carbon atoms, for example, 1 to 35 —CH₂— moieties, optionally wherein: if L comprises an alkylene comprising 1 to 35 —CH₂— moieties, then at least one, but no more than ten, —CH₂— moieties of L are independently replaced with a moiety selected from —C(═O)—, —C(═O)—NR⁴—, —NR⁴—C(═O)—, —C(═O)—
O—, —O—C(═O)—, —NR⁴—C(═O)—NR³—, —O—C(═O)—NR⁴—, —NR⁴—C(═O)—O—, —O—, —S—, and —NR⁴—, provided that the number of —CH₂— moieties of L is larger than the collective number of —C(═O)—, —C(═O)—NR⁴—, —NR⁴—C(═O)—, —C(═O)—O—, —O—C(═O)—, —NR⁴—C(═O)—NR³—, O—C(═O)—NR⁴—, —NR⁴—C(═O)—O—, —O—, —S—, and —NR— moieties of L, and provided there is at least one —CH₂— between each —C(═O)—, —C(═O)—NR⁴—, —NR⁴—C(═O)—, —C(═O)—O—, —O—C(═O)—, —NR⁴—C(═O)—NR³—, —O—C(═O)—NR⁴—, —NR⁴—C(═O)—O—, —O—, —S—, and —NR⁴— moiety of L;
R¹ and R² are each independently selected from H, alkyl, halo, hydroxyl, hydroxalkyl, carboxyl, acyl, ester, thioester, alkoxy, phosphoryl, amino, amido, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl;
R³ and R⁴ are each independently selected from H and alkyl; and
n is 1-5, preferably 1; 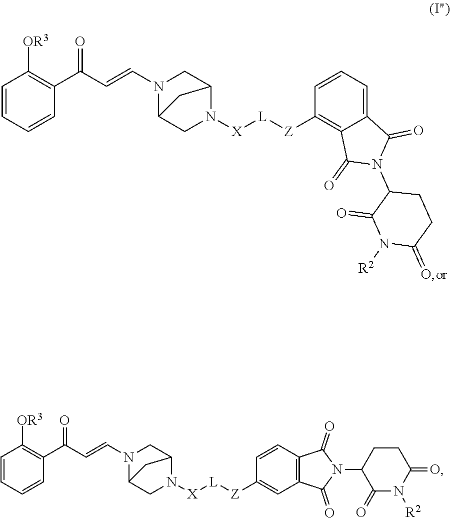
wherein
X is a bond, aryl, or heteroaryl;
L is an alkylene, alkenylene, or alkynylene chain comprising 1 to 35 carbon atoms, for example, 1 to 35 —CH₂— moieties,
optionally wherein:
at least one, but no more than ten, —CH₂— moieties of L are independently replaced with a moiety selected from —C(═O)—, —C(═O)—NR³— —NR³C(═O)—, —C(═O)—O—, —O—C(═O)—, —NR₃—C(═O)—NR³, —O—C(═O)—NR³, —NR³—C(═O)—O—, —O—, —S—, and —NR³, provided the number of —CH₂— moieties of L is larger than the collective number of —C(═O)—, —C═O)—NR³—, —NR³—C(═O)—, —C(═O)—O—, —O—C(═O)—, —NR³—C(═O)—NR³—, —O—C(═O)—NR³—, —NR³C(═O—O—, —O—, —S—, and —NR₃— moieties of L, and
provided there is at least one —CH₂— between each —C(═O)—, —C(═O)—NR₃—, —NR³—C(═O)—, —C(═O)—O—, —O—C(═O)—, NR³—C(═O)—NR³—, —O—C(═O)—NR³—, —NR³—C(═O)—O—, —O—, —S—, and —NR³— moiety of L,
Z-L is —CH₂-L, —O—CH₂-L, or —NR³—CH₂-L; and
R¹, R², and R³ are each independently selected from H and alkyl.

30. The method of claim 29, wherein the bifunctional compound is selected from: 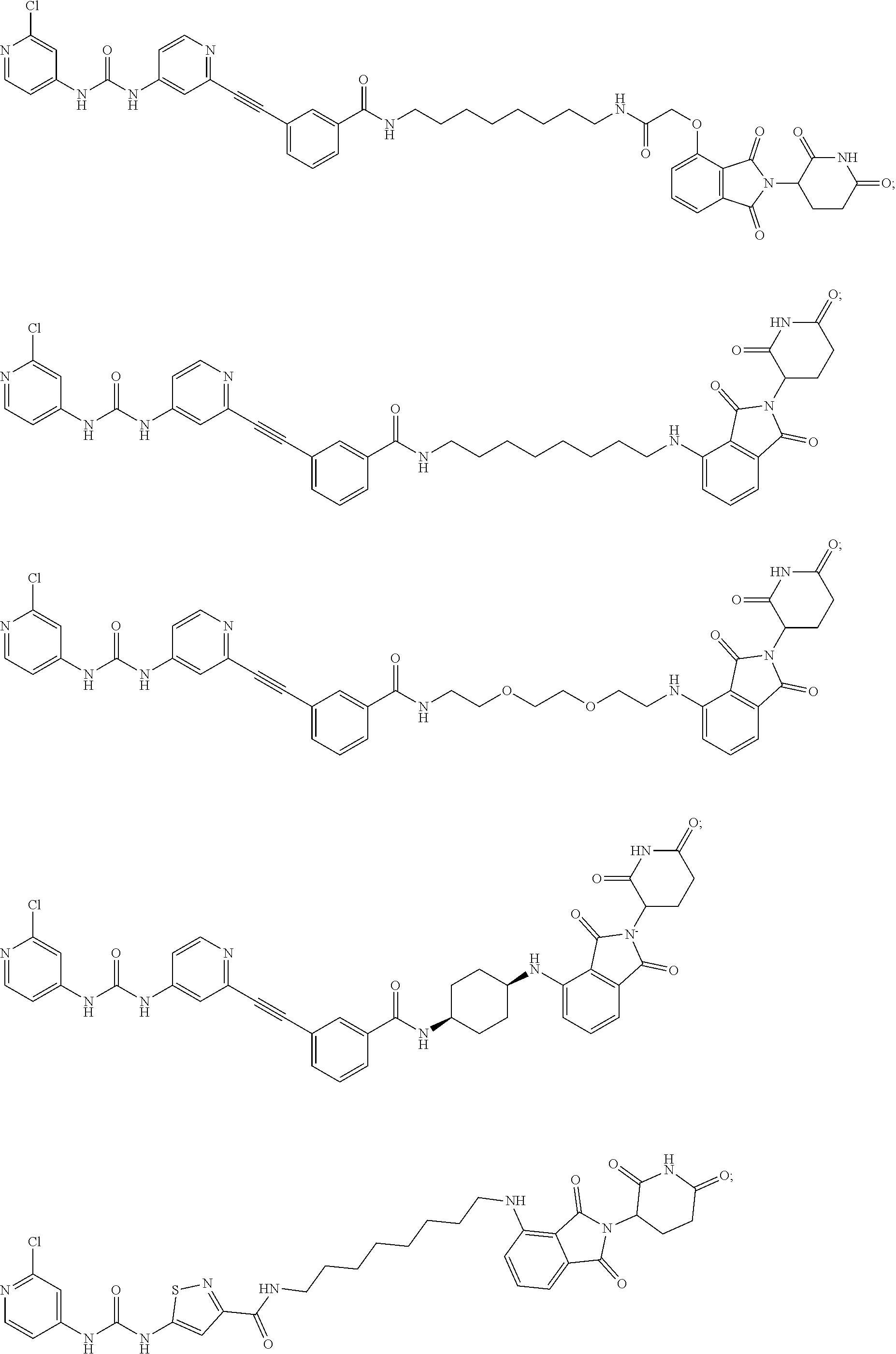 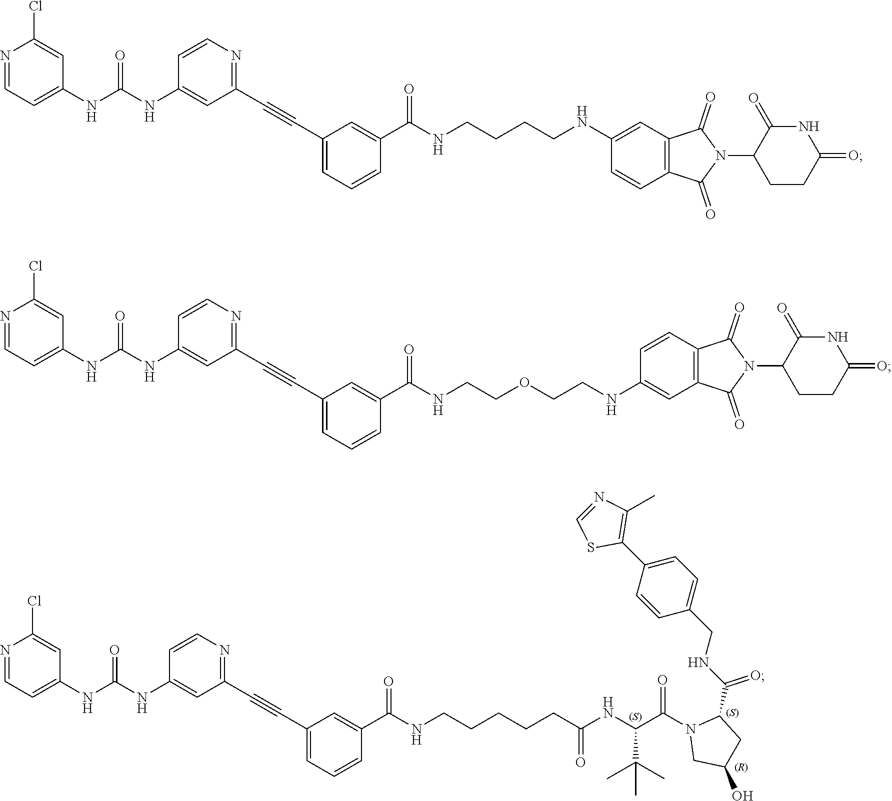 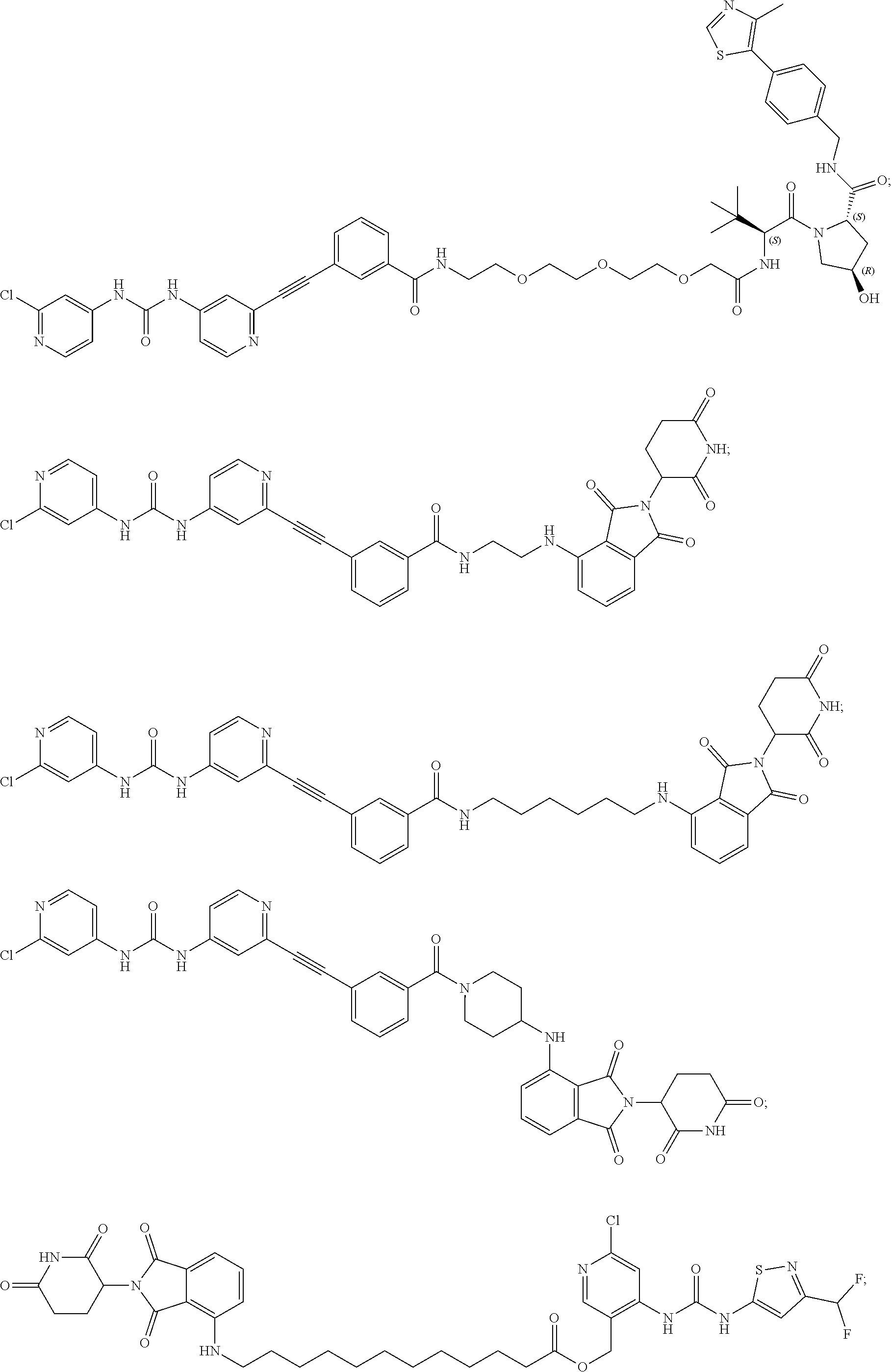 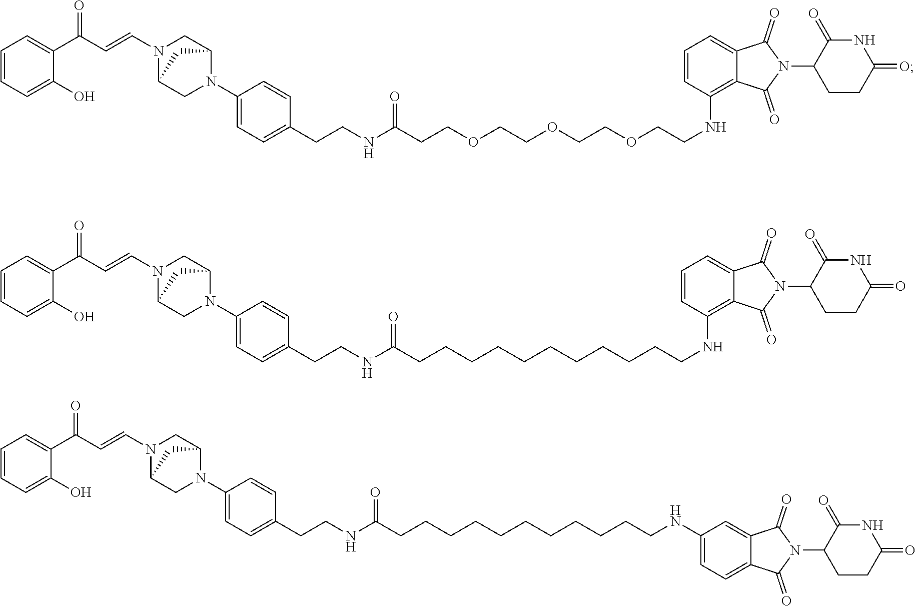 [SMILES: O=C(CCOCCOCCOCCNc1cccc2c1C(=O)N(C1CCC(=O)NC1=O)C2=O)NCCc1ccc(N2C[C@@H]3C[C@H]2CN3/C=C/C(=O)c2ccccc2O)cc1 ; O=C(CCCCCCCCCCCNc1cccc2c1C(=O)N(C1CCC(=O)NC1=O)C2=O)NCCc1ccc(N2C[C@@H]3C[C@H]2CN3/C=C/C(=O)c2ccccc2O)cc1 ; O=C(CCCCCCCCCCCNc1ccc2c(c1)C(=O)N(C1CCC(=O)NC1=O)C2=O)NCCc1ccc(N2C[C@@H]3C[C@H]2CN3/C=C/C(=O)c2ccccc2O)cc1] 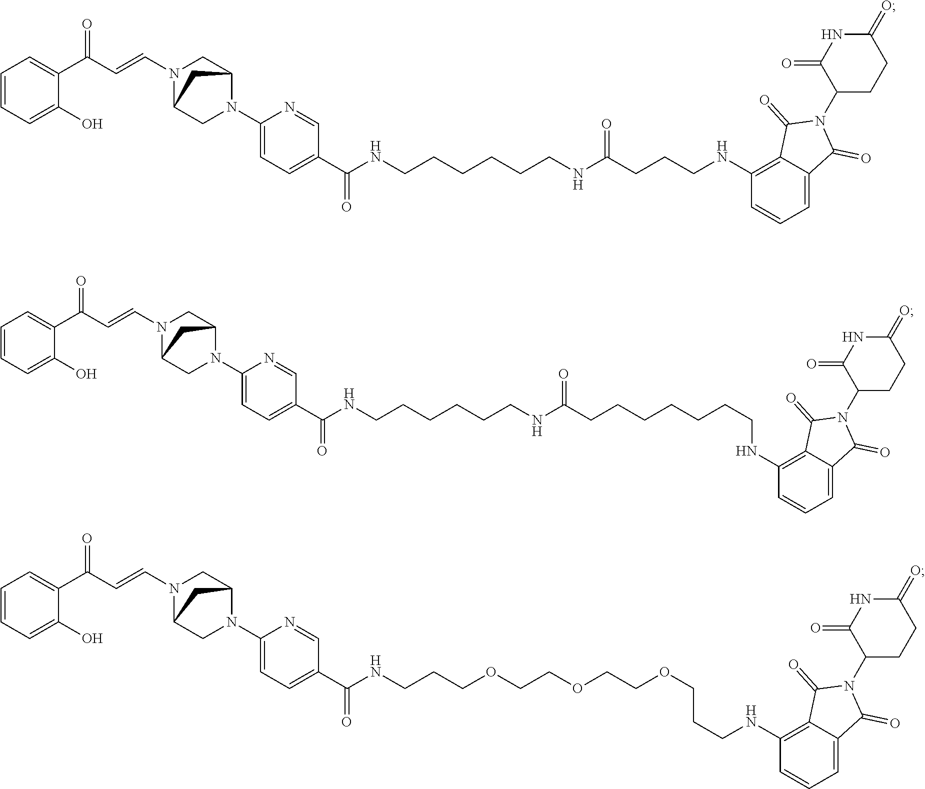 [SMILES: O=C(CCCNc1cccc2c1C(=O)N(C1CCC(=O)NC1=O)C2=O)NCCCCCCNC(=O)c1ccc(N2C[C@H]3C[C@@H]2CN3/C=C/C(=O)c2ccccc2O)nc1 ; O=C(CCCCCCCNc1cccc2c1C(=O)N(C1CCC(=O)NC1=O)C2=O)NCCCCCCNC(=O)c1ccc(N2C[C@H]3C[C@@H]2CN3/C=C/C(=O)c2ccccc2O)nc1 ; O=C1CCC(N2C(=O)c3cccc(NCCCOCCOCCOCCCNC(=O)c4ccc(N5C[C@H]6C[C@@H]5CN6/C=C/C(=O)c5ccccc5O)nc4)c3C2=O)C(=O)N1] 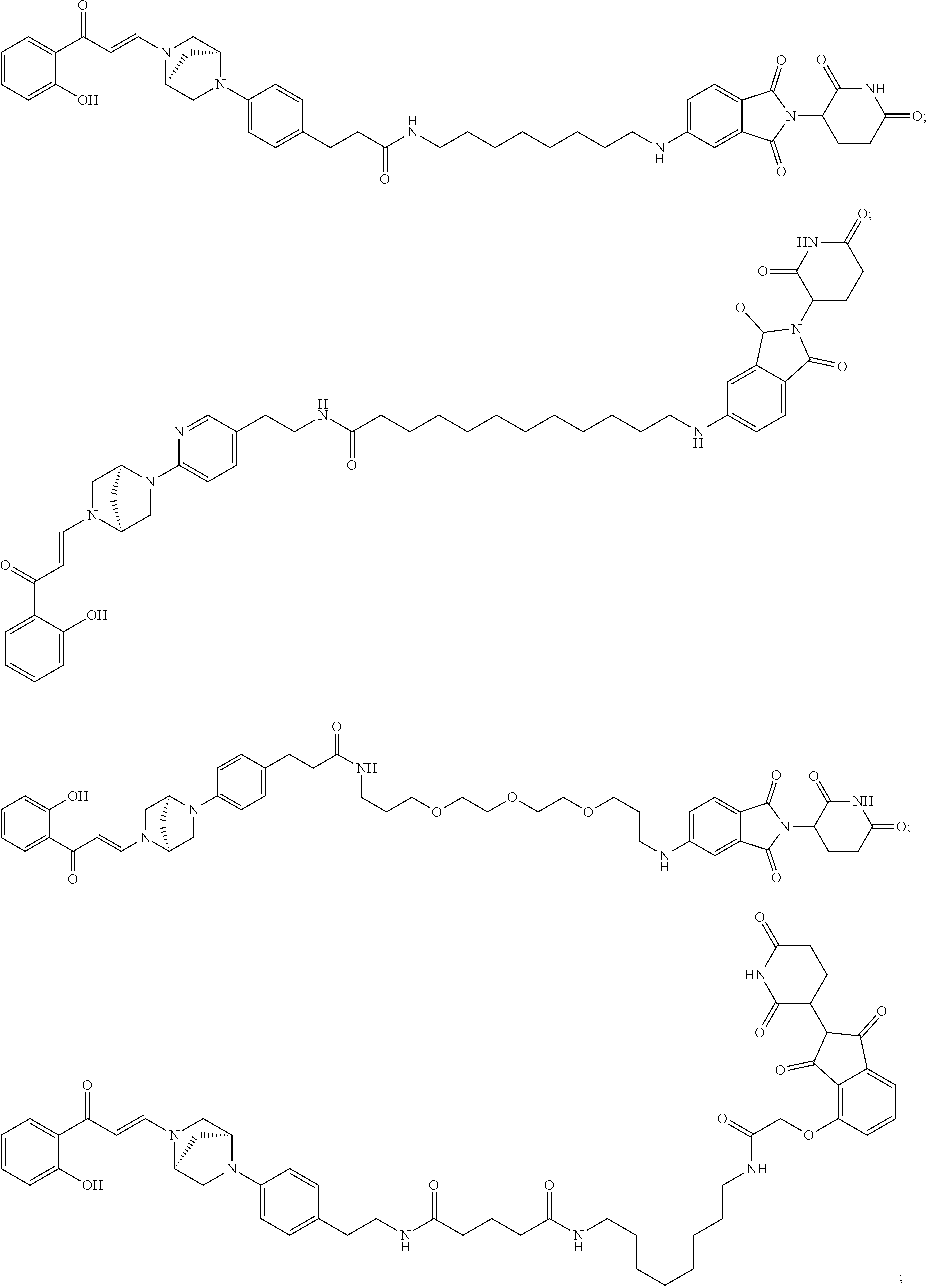 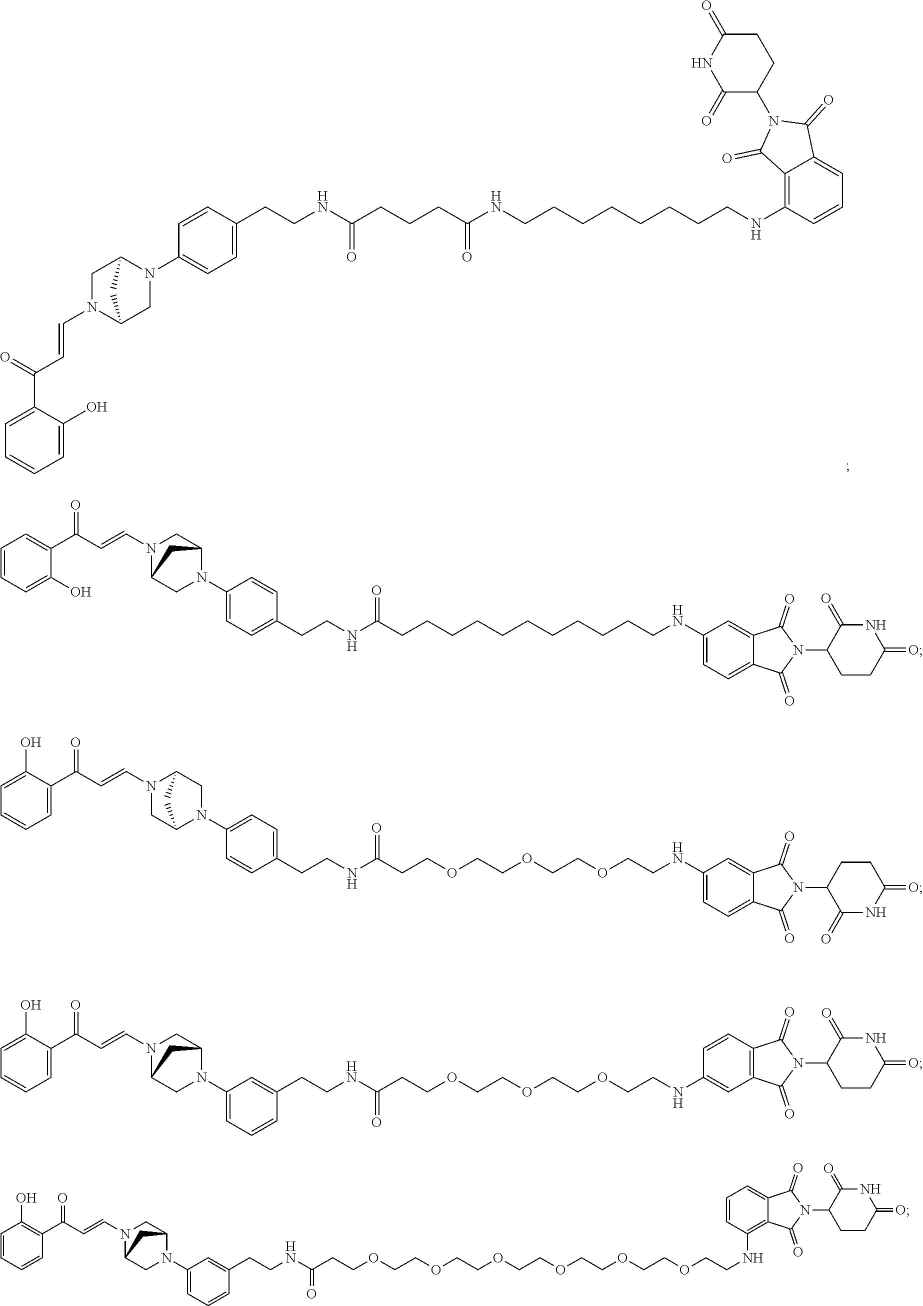 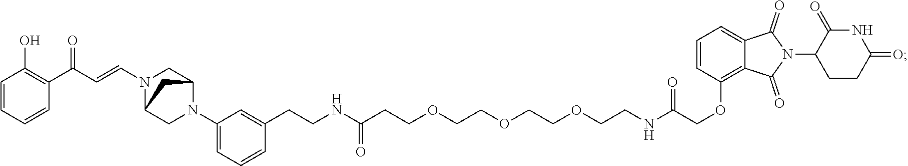 [SMILES: O=C(CCOCCOCCOCCNC(=O)COc1cccc2c1C(=O)N(C1CCC(=O)NC1=O)C2=O)NCCc1cccc(N2C[C@H]3C[C@@H]2CN3/C=C/C(=O)c2ccccc2O)c1] 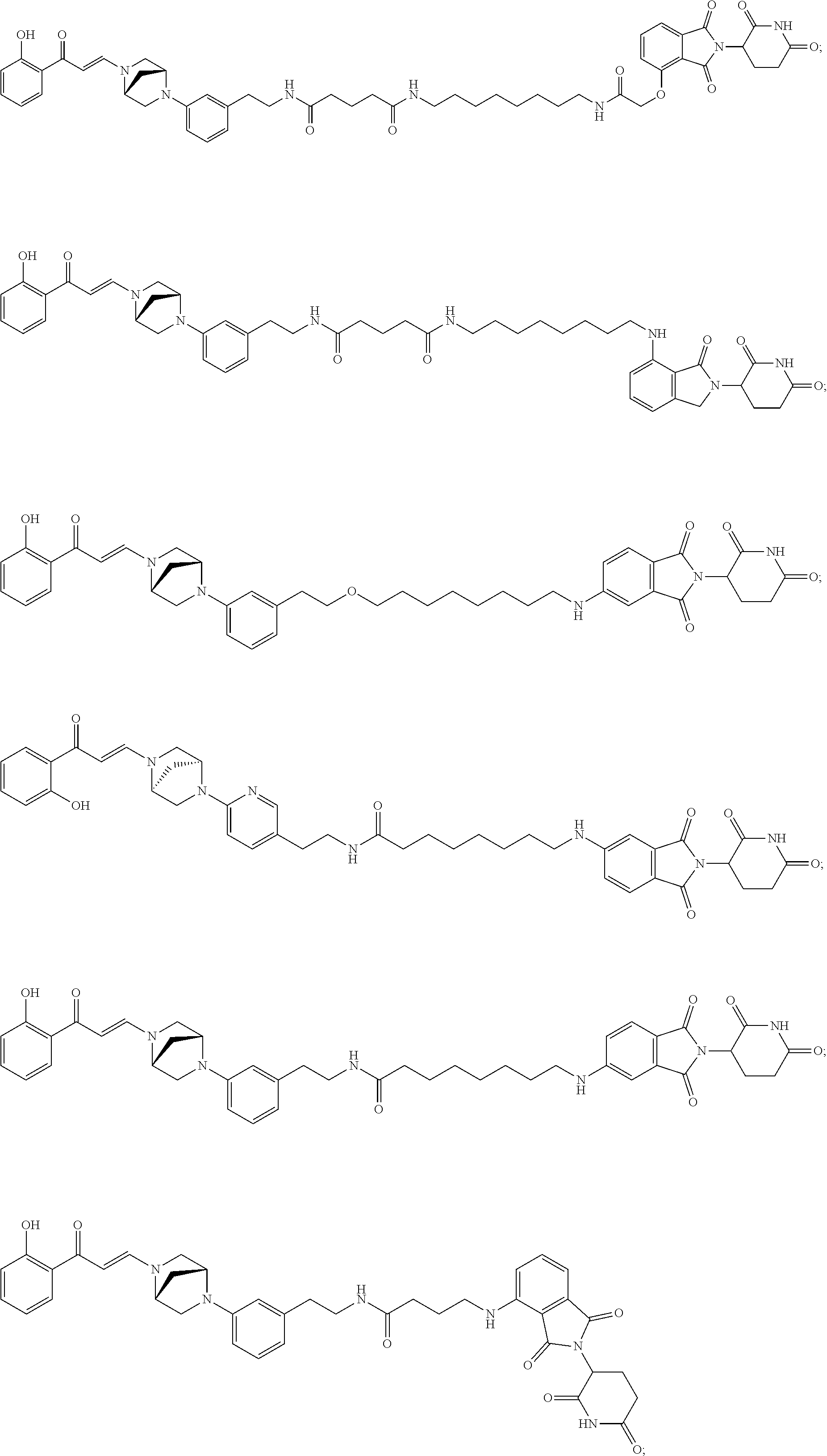 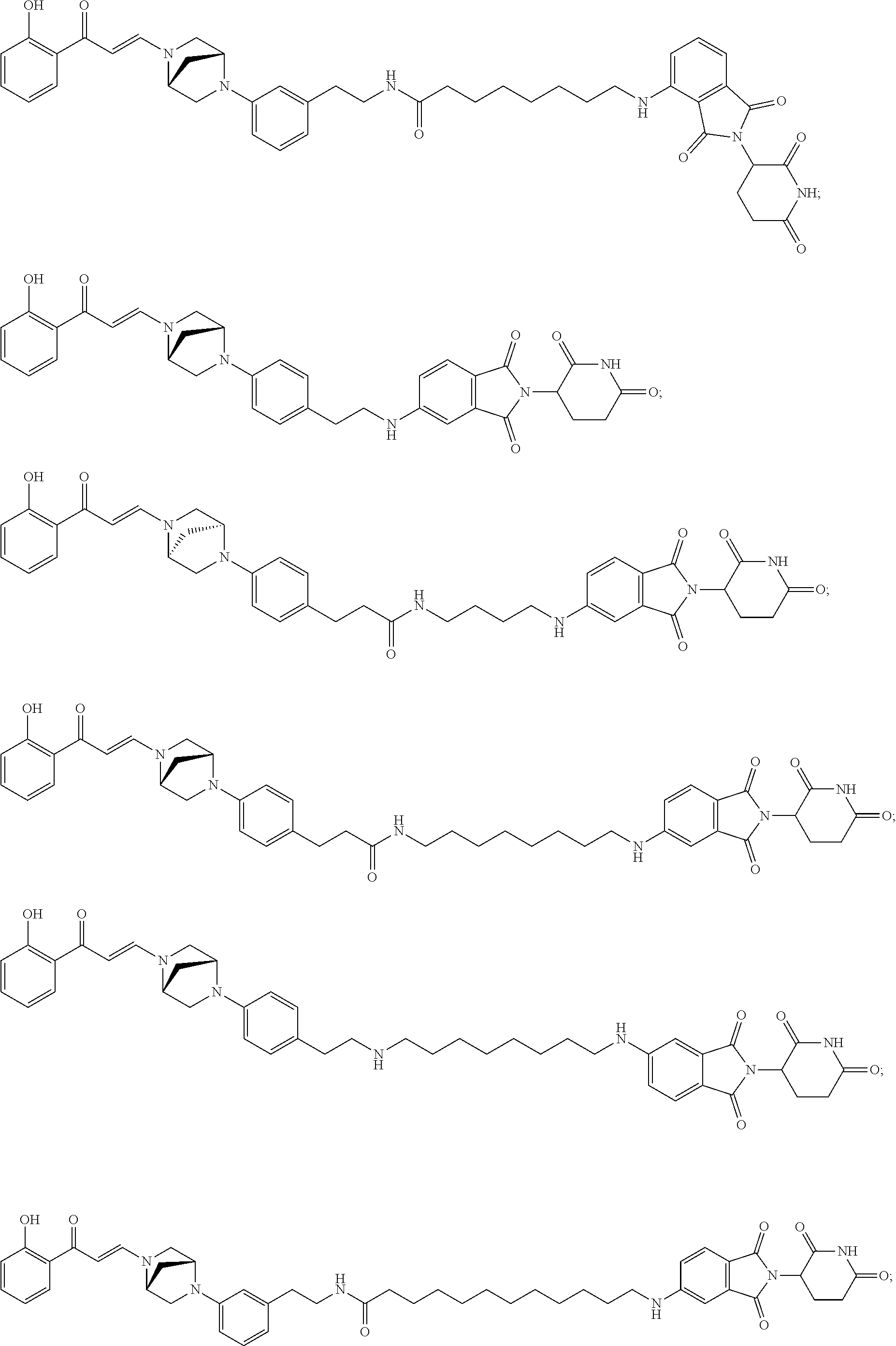 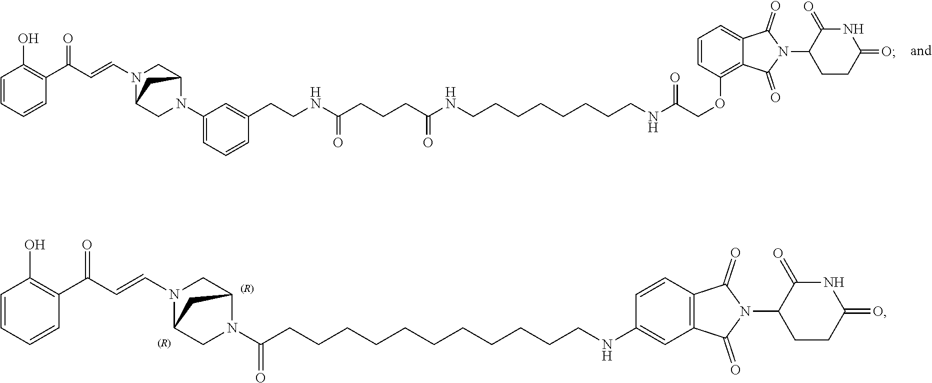
or a pharmaceutically acceptable salt or stereoisomer thereof.

31. The method of claim 29, wherein the diffuse midline glioma harboring the H3K27M mutation is diffuse intrinsic pontine glioma (DIPG).

32. The method of claim 29, further comprising administering to the subject a therapeutically effective amount of an additional pharmaceutical agent in combination with the bifunctional compound or pharmaceutically acceptable salt or stereoisomer thereof.

33. The method of claim 32, wherein the additional pharmaceutical agent is B Lymphoma Mo-MLV Insertion Region 1 Homolog (BMI1) inhibitor.

34. The method of claim 33, wherein the BMI1 inhibitor is PTC028, PTC209, PTC596, or QW24.

35. The bifunctional compound of claim 4, wherein R₁ is Cl or F.
